(19) United States
(12) Patent Application Publication
(10) Pub. No.: US 2015/0038357 A1
(43) Pub. Date: Feb. 5, 2015

(54) PROGNOSIS FOR GLIOMA

(71) Applicants: UNIVERSITE MONTPELLIER 2 SCIENCES ET TECHNIQUES, Montpellier Cedex (FR); INSTITUT CURIE, Paris (FR); INSERM, Paris (FR); CENTER HOSPITALIER UNIVERSITAIRE DE MONTPELLIER, Montpellier Cedex 5 (FR); CENTER NATIONAL DE LA RECHERCHE SCIENTIFIQUE, Paris Cedex 16 (FR); UNIVERSITYE MONTPELLIER 1, Montpellier Cedex 2 (FR)

(72) Inventors: Dominique Joubert, Sete (FR); Luc Bauchet, Clapiers (FR); Jean-Philippe Hugnot, Montpellier (FR); Ivan Bieche, Suresnes (FR); Rosette Lidereau, Gennevilliers (FR); Thierry Reme, Sainte Croix De Quintillargues (FR); Hugues Duffau, Montpellier (FR); Valerie Rigau, Mauguio (FR)

(21) Appl. No.: 14/350,086

(22) PCT Filed: Oct. 1, 2012
(86) PCT No.: PCT/EP2012/069387

§ 371 Date: Apr. 7, 2014

Related U.S. Application Data
(60) Provisional application No. 61/544,353, filed on Oct. 7, 2011.

(30) Foreign Application Priority Data
Oct. 7, 2011 (EP) 11306307.7

(51) Int. Cl. C12Q 1/68; G06F 19/00; G06F 19/20

(52) U.S. Cl. CPC C12Q 1/6886; G06F 19/20; G06F 19/3431; C12Q 2600/118; C12Q 2600/158
USPC 506/9; 506/16; 702/19

(57) ABSTRACT

Disclosed is a method of determining the survival prognosis of a patient afflicted by a glioma. The method includes assessing the level of expression of one or more specific gene in cells of the glioma.

### TECHNICAL FIELD

The present invention relates generally to methods and materials for use in providing a prognosis for patients afflicted by glioma.

### BACKGROUND ART

### Gliomas

Gliomas are tumors that originate from brain or spinal cord, in particular from glial cells or their progenitors. No underlying cause has been identified for the majority of gliomas. The only established risk factor is exposure to ionizing radiation. Just few percents of patients with gliomas have a family history of gliomas. Some of these familial cases are associated with rare genetic syndromes, such as neurofibromatosis types 1 and 2, the Li-Fraumeni syndrome (germ-line p53 mutations associated with an increased risk of several cancers), and Turcot's syndrome (intestinal polyposis and brain tumors). However, most familial cases have no identified genetic cause.

The incidence rate of the overall category glioma was 6.04 per 100,000 person-years, in US, for years 2004 to 2007 (CBTRUS 2011, http://www.cbtrus.org/2011-NPCR-SEER/WEB-0407-Report-3-3-2011.pdf).

Symptoms of gliomas depend on which part of the central nervous system is affected. A brain glioma can cause seizures, headaches, nausea and vomiting (as a result of increased intracranial pressure), mental status disorders, sensory-motor deficits, etc. A glioma of the optic nerve can cause visual loss. Spinal cord gliomas can cause pain, weakness, numbness in the extremities, paraplegia, tetraplegia, etc. Gliomas do not metastasize by the bloodstream, but they can spread via the cerebrospinal fluid and cause “drop metastases” to the spinal cord.

A child who has a subacute disorder of the central nervous system that produces cranial nerve abnormalities, long-tract signs, unsteady gait, and some behavioral changes is most likely to have a brainstem glioma.

Treatment for brain gliomas depends on the location, the cell type and the grade of malignancy. Histological diagnosis is mandatory, except in rare cases where biopsy or surgical resection is too dangerous. Often, treatment is a combined approach, using surgery, radiation therapy, and chemotherapy. The choice of treatments depends mainly on the histological study including the grading of the tumor. But unfortunately, the histological grading remains partly subjective and not always reproducible. Therefore, it is essential to define most relevant biological criteria to better adapt the treatments.

### Classification and Treatment of Gliomas

Conventionally, gliomas are classified by cell type, and by grade.

Gliomas are named according to the specific type of cell they share histological features with, but not necessarily originate from. The main types of gliomas are:
Astrocytomas—astrocytes (glioblastoma multiforme is the most common astrocytoma in adult and the most frequent malignant primitive brain tumor).
Oligodendrogliomas—oligodendrocytes.
Mixed gliomas, such as oligoastrocytomas, contain cells from different types of glia (astrocytes and oligodendrocytes).
Ependymomas—ependymal cells.

Gliomas are further categorized according to their grade, which is determined by pathologic evaluation of the tumor. Of numerous grading systems in use for gliomas, the most common is the World Health Organization (WHO) grading system, under which tumors are graded from I (least advanced disease—best prognosis) to IV (most advanced disease—worst prognosis). Ependymomas are specific kind of gliomas.

The classification (for astrocytomas, oligodendrogliomas and mixed tumors) is as follows:
Pilocytic astrocytoma is the most frequent grade I gliomas, mainly relevant to children and prognostis is very good when tumor could be totally resected.
Grade II gliomas are well-differentiated (not anaplastic) but not benign tumors.

They move inexorably toward anaplastic transformation, but the time to anaplastic transformation varies greatly from patient to patient. Survival varies also from patient to patient and the median overall survival is approximately 8 to 10 years.
Grade III gliomas are anaplastic. The prognosis is worse with an overall median survival of approximately 3 years.
Grade IV gliomas (Glioblastoma multiforme) are the most malignant primary central nervous system tumors with an overall survival of less than 1 year in population base-studies.

Moreover, gliomas are often subdivided or classified in low grade gliomas (grade I and II) and high gliomas (grade III and IV). As new treatments (surgery with functional and imaging techniques, conformational and new techniques for radiotherapy, new drugs for chemotherapy and targeted therapies, etc.) are now available, it is clearly demonstrated that treatments can influence the survival of glioma patients. In addition, treatments and oncological care for low grade glioma and high grade glioma pateints are very different.

So, it is important, to correctly determine the type of glioma that afflicts a subject, in order to both determine the prognosis, and to propose an adapted therapy.

Treatments for low grade glioma aim at avoiding the malignity increase as long as possible while preserving the patient's quality of life. However the management of patients with low grade glioma is a challenge as these tumors are clearly an heterogenous group with different evolution especially regarding the risk of anaplastic transformation occurring either rapidly or long after diagnosis. Indeed, these tumours will ineluctably degenerate toward anaplastic glioma within 5-10 years which then leads to the death of the patient rapidly. However approximately 10-20% of patients have a more rapid tumoral growth and transform to anaplasia more rapidly. This poses important dilemmas for defining the best therapeutic approach (exeresis with or without chemotherapy). There is currently no definitive criteria to classify a low grade lesion as at high risk or low risk to relapse and/or rapid progression. The neuropathological classification based on histology and immunohistochemistry data is unfortunately unreliable and there is a considerable level of discrepancy between neuropathologists for the same tumor sample (Prayson R A, J Neurol Sci, 2000, 175(1), 33-9). Clearly, the definition of novel biological criteria to implement the identification of high-risk patients that would need more aggressive adjuvant treatments would be a major breakthrough in the field.

### Background Art Relating to Methods for Diagnosis and Prognosis of Gliomas

The international application WO 2008/031165 discloses methods for the diagnosis and prognosis of tumours of the central nervous system, including of the brain, particularly tumours of neuroepithelial tissue (glioma(s)). In particular, WO/2008/031165 relates to a method comprising determining the expression of at least one gene selected from the group consisting of IQGAPI, Homer 1, and CIQLI or determining the expression of at least two genes selected from the group consisting of IQGAPI, Homer 1, IGFBP2, and CIQLI in a biological sample from an individual.

The international application WO 2008/067351 discloses a method for diagnosing the presence of a glioma tumor in a mammal, wherein the method comprises comparing the level of expression of PIK3R3 polypeptide or nucleic acid encoding a PIK3R3 polypeptide. This application discloses a method for diagnosing the severity of a glioma tumor in a mammal, wherein the method comprises: (a) contacting a test sample comprising cells from said glioma tumor or extracts of DNA, RNA, protein or other gene product(s) obtained from the mammal with a reagent that binds to the PIK3R3 polypeptide or nucleic acid encoding PIK3R3 polypeptide in the sample, (b) measuring the amount of complex formation between the reagent with the PIK3R3-encoding nucleic acid or PIK3R3 polypeptide in the test sample, wherein the formation of a high level of complex, relative to the level in known healthy sample of similar tissue origin, is indicative of an aggressive tumor.

The international application WO 2008/021483 discloses a method for diagnosing a disease state or a phenotype or predicting disease therapy outcome in a subject, said method comprising: a) obtaining a sample from a subject; b) screening for a simultaneous aberrant expression level of two or more markers in the same cell from the sample; c) scoring the expression level as being aberrant when the expression level detected is above or below a certain threshold coefficient; wherein the detection threshold coefficient is determined by comparing the expression levels of the samples obtained from the subjects to values in a reference database of sample phenotypes obtained from subjects with either a known diagnosis or known clinical outcome after therapy, wherein the presence of an aberrant expression level of two or more markers in individual cells and presence of cells aberrantly expressing two or more such markers is indicative of a disease diagnosis or prognosis for therapy failure in the subject.

The international application WO 2005/028617 discloses that an increase of the α4 chain-containing Laminin-8 correlates with poor prognosis for patients with brain gliomas.

Certain other genes described below have also been described in publications concerning glioma: CHI3L1 (Clin Cancer Res. 2005 May 1; 11(9):3326-34 & PLoS One. 2010 Sep. 3; 5(9):e12548); BIRC5 (J Clin Neurosci. 2008 November; 15(11):1198-203 Epub 2008 Oct. 5 & J. Clin Oncol. 2002 Feb. 15; 20(4):1063-8; VIM (Acta Neuropathol. 1998 May; 95(5):493-504); TNC (Cancer. 2003 Dec. 1; 98(11):2430); AURKA and DLL3 (PLoS One. 2010 Sep. 3; 5(9):e12548); and KI67 (Clin Neuropathol. 2002 November-December; 21(6):252-7, Pathol Res Pract. 2002; 198(4):261-5). Additionally BMP2 has been proposed as a serum marker for glioblastomas (J Neurooncol. 2011 March; 102(1):71-80.) and increased levels of BMP2 in grade 3-4 versus grade 1-2 gliomas has been reported (Xi Bao Yu Fen Zi Mian Yi Xue Za Zhi. 2009 July; 25(7):637-9.). BMP2 expression has also been shown to be increased in 1p19q codeletion gliomas (Mol Cancer. 2008 May 20; 7:41.) and implicated in differential survival between grade 3 gliomas and glioblastomas (Cancer Res. 2004, 64:6503-6510).

However, none of the above methods, or other methods belonging to the art, takes account of the possible miss-classification of tumors, and therefore the possibility to miss-prognose patient, or to provide to patients inappropriate therapy.

The purpose of the invention is to overcome these inconveniencies.

One aim of the invention is to provide a new efficient phenotypic or prognostic method of gliomas. Another aim of the invention is to provide compositions for carrying out the phenotypic or prognostic method. Another aim is to provide a kit for prognosing gliomas.

Other objects and aims are described herein. Furthermore it can be seen that the identification of genes, or sets of genes, the expression of which can be used in the classification or prognosis of gliomas and\or the devising of appropriate treatment strategies for gliomas, would provide a contribution to the art.

### DISCLOSURE OF THE INVENTION

The present inventors have identified genes and gene expression signatures which can be usefully employed in the classification or prognosis of gliomas and\or the devising of appropriate treatment strategies for gliomas. Such genes, or in some cases combinations of genes, have not previously been shown to have utility in diagnosing or prognosing glioma survival.

The phenotype can, if desired, be used to supplement other diagnostic or prognostic markers, or clinical assessment. A preferred phenotype is a predicted survival.

The relevant gene expression may also be used as a biomarker for choosing or monitoring specific therapeutic regimes and chemotherapeutic combinations.

Thus in one aspect the invention provides a method of predicting the survival prognosis of a patient afflicted by a glioma, the method comprising assessing the level of expression of a gene or genes of Table 10 in cells of the glioma.

In another aspect of the invention there is provided use of any one (or more) of the genes of Table 10 for determining a survival prognosis for a patient afflicted by a glioma:

TABLE 10

| SEQ ID | Gene name |
|---|---|
| SEQ ID NO: 3 | POSTN |
| SEQ ID NO: 4 | HSPG2 |
| SEQ ID NO: 6 | COL1A1 |
| SEQ ID NO: 7 | NEK2 |
| SEQ ID NO: 8 | DLG7 |
| SEQ ID NO: 9 | FOXM1 |
| SEQ ID NO: 11 | PLK1 |
| SEQ ID NO: 12 | NKX6-1 |
| SEQ ID NO: 13 | NRG3 |
| SEQ ID NO: 14 | BUB1B |
| SEQ ID NO: 18 | JAG1 |
| SEQ ID NO: 20 | EZH2 |
| SEQ ID NO: 21 | BUB1 |

Further information about these sequences is provided in the Tables and other disclosure below. As explained in detail hereinafter, the aspects and embodiments of the invention described and defined herein apply mutatis mutandis to variants of these genes also.

In general terms, and as described herein, underexpression of NRG3 may be associated with poor prognosis, while overexpression of the remaining genes in Table 10 may be associated with poor prognosis.

In one aspect the method may comprise the steps of obtaining a test sample comprising nucleic acid molecules from a sample of the glioma then determining the amount of the relevant mRNA in the test sample and optionally comparing that amount to a predetermined value.

As described in more detail below, levels of “expression” may be detected either from levels of nucleic acid or protein. For example protein may be detected in the cell membrane, the endoplasmic reticulum or the Golgi apparatus (by direct binding or by activity) or nucleic acid may be detected from mRNA encoding the relevant gene, either directly or indirectly (e.g. via cDNA derived therefrom). Put another way, the expression may be measured directly (e.g. using RT-PCT or microarrays) or indirectly (e.g. by proteomic analysis).

In one embodiment the method may comprise the steps of:

(a) contacting a sample of the glioma obtained from the patient with a binding agent that specifically binds to the encoded protein or relevant mRNA; and

(b) detecting the amount of protein or mRNA that binds to the binding agent,

(c) optionally comparing the amount of protein or mRNA to a predetermined cut-off value, and thereby making a determination about phenotype (e.g. prognosis)

As noted below, the sample will typically be the tumor itself.

In another aspect there is provided a method for determining a clinical phenotype (such as prognosis) for a patient afflicted by a glioma, which method comprises:

(i) assessing and preferably quantifying the expression level of one or more genes (e.g. a set of genes) in a sample from said patient,

(ii) comparing expression value or values obtained from step (i) with one or more reference expression values for each of said plurality of genes,

(iii) determining the clinical phenotype (e.g. prognosis) based on the comparison at (ii).

In this method the comparison at (ii) can provide a “gene signature” (e.g. based on aberrant expression of the genes).

The gene or genes may include any of those from Table 10, which genes have not previously been shown to have utility in diagnosing or prognosing glioma survival. In other embodiments of the invention described in more detail below, a plurality of genes may be selected from Table 1, which combination of genes has not previously been shown to have utility in diagnosing or prognosing glioma survival.

### Glioma

Preferably the glioma is a WHO grade 2 or grade 3 glioma.

Moreover, the Inventors have determined that the WHO classification in class 2 or 3 is not representative of the prognosis outcome, whereas the method according to the invention is representative of the prognosis outcome.

In the invention “WHO grade 2 or grade 3 glioma” corresponds to the World Health Organisation classification of glioma.

Biological sample according to the invention are commonly classified by histological techniques according to a common proceeding well known in the art.

### Biological Sample

“A biological sample of a subject afflicted by a WHO grade 2 or grade 3 glioma” corresponds to a sample originating from an individual afflicted by a grade 2 or grade 3 glioma, and is commonly essentially constituted by the tumor. This could be, for instance, a biopsy obtained after surgery. Biological samples according to the invention are commonly classified by histological techniques according to a common proceeding well known in the art.

Methods in which the Invention has Utility

By “method for determining the survival prognosis of said patient” or the like, it is meant in the invention that the method allows to predict the likely outcome of an illness, e.g. the outcome of grade 2 and grade 3 gliomas. More particularly, the prognosis method can evaluate the survival rate, said survival rate indicating the percentage of people, in a study, who are alive for a given period of time after diagnosis. This information allows the practitioner to determine if a medication is appropriated, and in the affirmative, what type of medication is more appropriate for the patient.

### Quantification of Genes

The measure of the expression utilised in the invention is a quantitative measure. In other words, for each gene, a value is obtained by techniques well known in the art.

In one preferred embodiment of the invention, the terms “determining the quantitative expression” of gene “I” means that the measure of the transcription product(s) of said gene, e.g. messenger RNA (mRNA), is evaluated, and quantified. In other words, in the invention, the amount of the transcript(s) of said gene is quantified. In other embodiments the expression can be determined indirectly based on derived nucleic acids, or polypeptide expression products.

Methods of determining quantitative expression are described in more detail hereinafter.

Thus in preferred embodiments described herein the quantitative value Qi, for a gene is therefore representative of the amount of molecule of mRNA, or the corresponding cDNA, expressed for said gene i in the biological sample of the patient.

“The quantitative value Qi, for a gene i” means, for instance, that for the gene 3 (i.e. gene SEQ ID NO: 3) the quantitative value measured will be Q3. This example applies mutatis mutandis for all the other genes of the group of 22 genes in Table 1, i.e Q1 for gene 1 (SEQ ID NO: 1), Q2 for gene 2 (SEQ ID NO: 2) . . . etc.

### Normalisation of Quantification of Genes

Generally speaking, the method used to measure the expression level of a gene i gives a “signal” representative of the raw amount of the gene i product in the biological sample. In order to correctly evaluate the real amount of said gene i product, the signal is compared to the “signal of a control gene”, said control gene being a gene for which the expression level never, or substantially never, varies whatsoever the conditions (normal or pathologic). The control genes commonly used are housekeeping genes such as actin, Glyceraldehyde-3 phosphate deshydrogenase (GAPDH), tubulin, Tata box binding protein (TBP). The use of such control genes to quantify expression of a gene of interest is well known in the art and does not per se form part of the present invention.

Thus at various points herein the term “quantitative raw expression value” or “Qri” may be used to describe a ‘normalised’ quantitative expression of a gene:

To obtain the Qri value for a determined gene i, the following formula can be applied:

$$Qri = {log}_{2} ( \frac{Si}{Sc} \times 1000 ) ,$$

wherein Si represents the signal obtained for a gene i, and Sc represents the signal obtained for the control gene, Si and Sc being obtained in the same biological sample, if possible during the same experiment.

This normalisation has particular value when the quantification relies on an amplification method such as PCR.

Thus, in summary, in methods of the invention, including step (i) as defined above, the expression level of the gene in the cells is preferably “normalised” to a standard gene e.g. a housekeeping gene as described herein. This so called normalised “raw expression value” may be referred to as “Qri” for gene “i” herein.

### Reference Expression Values

In the present invention the expression level of the gene or genes is compared to a reference value in order that a determination of phenotype (e.g. prognosis) can be made.

In certain embodiments of the present invention the reference expression value or values may be based on tissue (e.g. brain tissue) obtained from, by way of example:

(a) histologically normal tissue (same or different tissue) of the subject individual

(b) a similar or identical region of the brain of a second individual of known glioma status (e.g. normal, afflicted)

(c) a reference cell line

(d) an averaged value based on number of reference individuals.

In preferred embodiments the reference value or values are obtained from a cohort of reference patients afflicted by glioma.

By “reference patients” as it is defined in the invention is meant patients for which data regarding their survival, the evolution of their pathology, the treatment or surgery that they have received over many months or years are known.

These reference, or control, patients are regrouped in a panel called cohort. Thus the reference expression value may be determined from expression levels obtained from a reference database of sample phenotypes obtained from this cohort of subjects afflicted with glioma with either a known diagnosis or known clinical outcome after therapy.

Thus, preferably, in step (ii) of the method the expression level of the gene in the cells can be “centred” with respect to a mean-normalised expression of the gene in a plurality of corresponding reference samples from a cohort of glioma patients. Such a mean-normalised expression may be referred to herein as “Qci”.

Put another way, in methods of the invention it may be desired to define a quantitative expression value Qi for a gene I, which corresponds to the comparison between:
the quantitative raw expression value Qri measured for a gene i, in the biological sample of said subject, and
a Qci value corresponding to the mean of the quantitative expression values obtained for said gene i from each patient of a reference or control cohort of patients

The reference or control cohort may be composed of patients afflicted by the same glioma e.g. a WHO grade 2 or grade 3 glioma.

The Qi value can be calculated from Qi=Qri−Qci.

It will be appreciated therefore that in this step the “centred expression” may be positive (if the expression in the sample is higher than the reference mean, or “over-expressed” compared to the reference mean) or negative (if the expression in the sample is lower than the reference mean, or “under-expressed compared to the reference mean).

In step (ii) of the method above the normalised expression level of the gene in the cells may be scaled by reference to a deviation score based on the plurality of corresponding samples from the cohort of glioma patients. The “scaled centred” expression may be obtained by dividing the centred expression by the standard deviation.

The statistical relevance of preferred methods according to the invention is shown below and in the examples.

### Choice of Genes

In the present invention the genes described herein may be used to provide a “molecular signature” or “gene-expression signature”. Such a signature, as used herein refers, to two or more genes that are co-ordinately expressed in the glioma samples and which can be used to predict or model patients' clinically relevant information (e.g. prognosis, survival time, etc) as a function of the gene expression data.

Various genes and gene combinations which are preferred embodiments are described herein below in relations to combinations of SEQ ID NOs 1-22.

In some embodiments at least 1 gene from Table 10 is assessed.

In some embodiments at least 2 genes from Table 10 are assessed.

In some embodiments at least 3 genes from Table 10 are assessed.

In some embodiments at least 2 or 3 genes from the 22 genes of Table 1 are assessed, which combination preferably includes at least 1 gene from Table 10

By “at least 2 or 3 genes belonging to a group of 22 genes”, it is meant in the invention that 2 or 3, or 4, or 5, or 6, or 7, or 8, or 9, or 10, or 11, or 12, or 13, or 14, or 15, or 16, or 17, or 18, or 19, or 20, or 21, or 22 genes can be used.

In one embodiment the invention comprises assessing at least 2 genes belonging to a group of 22 genes as described herein, which combination preferably includes at least 1 gene from Table 10.

In one embodiment the invention comprises assessing at least 3 genes belonging to a group of 22 genes as described herein, which combination preferably includes at least 1 gene from Table 10.

Preferably at least 3 genes belonging to the group of 22 genes is assessed.

Preferably at least SEQ ID NO: 3 (POSTN) is assessed.

In one embodiment the first step of a method according to the invention corresponds to a step of measuring and quantifying the expression level of at least 3 genes comprising or being constituted by the nucleic acid sequences as set forth in SEQ ID NO: 1 to 3, said at least 3 genes belonging to a group of 22 genes comprising or being constituted by the nucleic acid sequences as set forth in SEQ ID NO: 1 to 22.

Thus, by way of example, the measure of the expression level of the genes represented by SEQ ID NO: 1, SEQ ID NO: 2 and SEQ ID NO: 3 is sufficient to carry out the method according to the invention.

Thus one condition imposed on this embodiment of the method is that genes comprising or being constituted by the nucleic acid molecules as set forth in SEQ ID NO: 1, SEQ ID NO: 2 and SEQ ID NO: 3 are always present in anyone of the combinations mentioned above.

For instance, if 4 genes are considered, 19 combinations are possible:

SEQ ID NO: 1, SEQ ID NO: 2, SEQ ID NO: 3 and SEQ ID NO: 4,

SEQ ID NO: 1, SEQ ID NO: 2, SEQ ID NO: 3 and SEQ ID NO: 5,

SEQ ID NO: 1, SEQ ID NO: 2, SEQ ID NO: 3 and SEQ ID NO: 6,

SEQ ID NO: 1, SEQ ID NO: 2, SEQ ID NO: 3 and SEQ ID NO: 7,

SEQ ID NO: 1, SEQ ID NO: 2, SEQ ID NO: 3 and SEQ ID NO: 8,

SEQ ID NO: 1, SEQ ID NO: 2, SEQ ID NO: 3 and SEQ ID NO: 9,

SEQ ID NO: 1, SEQ ID NO: 2, SEQ ID NO: 3 and SEQ ID NO: 10,

SEQ ID NO: 1, SEQ ID NO: 2, SEQ ID NO: 3 and SEQ ID NO: 11,

SEQ ID NO: 1, SEQ ID NO: 2, SEQ ID NO: 3 and SEQ ID NO: 12,

SEQ ID NO: 1, SEQ ID NO: 2, SEQ ID NO: 3 and SEQ ID NO: 13,

SEQ ID NO: 1, SEQ ID NO: 2, SEQ ID NO: 3 and SEQ ID NO: 14,

SEQ ID NO: 1, SEQ ID NO: 2, SEQ ID NO: 3 and SEQ ID NO: 15,

SEQ ID NO: 1, SEQ ID NO: 2, SEQ ID NO: 3 and SEQ ID NO: 16,

SEQ ID NO: 1, SEQ ID NO: 2, SEQ ID NO: 3 and SEQ ID NO: 17,

SEQ ID NO: 1, SEQ ID NO: 2, SEQ ID NO: 3 and SEQ ID NO: 18,

SEQ ID NO: 1, SEQ ID NO: 2, SEQ ID NO: 3 and SEQ ID NO: 19,

SEQ ID NO: 1, SEQ ID NO: 2, SEQ ID NO: 3 and SEQ ID NO: 20,

SEQ ID NO: 1, SEQ ID NO: 2, SEQ ID NO: 3 and SEQ ID NO: 21, and

SEQ ID NO: 1, SEQ ID NO: 2, SEQ ID NO: 3 and SEQ ID NO: 22,

The skilled person will know how to determine all the combinations of at least 3 genes among 22 genes encompassed by the invention.

According to the invention, the 22 genes and their corresponding SEQ ID are represented in the following table 1:

| | Gene | |
|---|---|---|
| SEQ ID | name | Access number (Ensembl) |
| SEQ ID NO: 1 | CHI3L1 | ENSG00000133048 |
| SEQ ID NO: 2 | IGFBP2 | ENSG00000115457 |
| SEQ ID NO: 3 | POSTN | ENSG00000133110 |
| SEQ ID NO: 4 | HSPG2 | ENSG00000142798 |
| SEQ ID NO: 5 | BMP2 | ENSG00000125845 |
| SEQ ID NO: 6 | COL1A1 | ENSG00000108821 |
| SEQ ID NO: 7 | NEK2 | ENSG00000117650 |
| SEQ ID NO: 8 | DLG7 | ENSG00000126787 |
| SEQ ID NO: 9 | FOXM1 | ENSG00000111206 |
| SEQ ID NO: 10 | BIRC5 | ENSG00000089685 |
| SEQ ID NO: 11 | PLK1 | ENSG00000166851 |
| SEQ ID NO: 12 | NKX6-1 | ENSG00000163623 |
| SEQ ID NO: 13 | NRG3 | ENSG00000185737 |
| SEQ ID NO: 14 | BUB1B | ENSG00000156970 |
| SEQ ID NO: 15 | VIM | ENSG00000026025 |
| SEQ ID NO: 16 | TNC | ENSG00000041982 |
| SEQ ID NO: 17 | DLL3 | ENSG00000090932 |
| SEQ ID NO: 18 | JAG1 | ENSG00000101384 |
| SEQ ID NO: 19 | KI67 | ENSG00000148773 |
| SEQ ID NO: 20 | EZH2 | ENSG00000106462 |
| SEQ ID NO: 21 | BUB1 | ENSG00000169679 |
| SEQ ID NO: 22 | AURKA | ENSG00000087586 |

Table 1 represents the genes according to the invention, and their corresponding SEQ ID, and the corresponding Access number in the Ensembl database (http://www.ensembl.org/index.html).

Advantageously, the invention relates to the method as defined above which comprises assessing a set of genes including or consisting of at least 2 or at least 3 genes belonging to a group of 22 genes of Table 1, including at least 1 gene from Table 10.

In general terms, and as described herein, underexpression of APOD, BMP2, DLL3, NRG3 and TACSTD1 may be associated with good prognosis, while overexpression of the remaining genes in Table 1 may be associated with poor prognosis.

Advantageously, the invention relates to a method for determining, preferably in vitro or ex vivo, from a biological sample of a subject afflicted by a WHO grade 2 or grade 3 glioma, the survival prognosis of said patient,

### Said Method Comprising:

determining the quantitative expression value Qi for each gene of a set comprising at least 3 genes belonging to a group of 22 genes, said 22 genes comprising or being constituted by the respective nucleic acid sequences SEQ ID NO: 1 to 22,
wherein said at least 3 genes comprise or are constituted by the respective nucleic acid sequences SEQ ID NO: 1 to 3,
establishing
   a first product P₁i for each of said at least 3 genes, between the respective Qi values obtained above for each said at least 3 genes and a first value V₁i, and
   a second product P₂i for each of said at least 3 genes, between the respective Qi values obtained above for each said at least 3 genes and a second value V₂i,
wherein
   said first value Vii corresponds to the shrunken centroid value for a gene i obtained from reference patients having a WHO grade 2 or grade 3 glioma, said reference patients having a median survival higher than 4 years, and
   said second value V2i corresponds to the shrunken centroid value for a gene i obtained from reference patients having a WHO grade 2 or grade 3 glioma, said reference patients having a median survival lower than 4 years,
      said patients having a WHO grade 2 or grade 3 glioma with a median survival lower or higher than 4 years belonging to a reference cohort of patients afflicted by either a WHO grade 2 or a WHO grade 3 glioma,
determining the survival rate of said patient as follows:
   if the sum of the P₁i products of each of said at least 3 genes is higher than the sum of the P₂i products of each of said at least 3 genes, then said subject has a median survival higher than 4 years, and
   if the sum of the P₁i products of each of said at least 3 genes is lower than or equal to the sum of the P₂i products of each of said at least 3 genes, then said subject has a median survival lower than 4 years.

According to the invention, the product P₁i is obtained from the following formula:
P₁i=Qi×V₁i, wherein V₁i corresponds to the shrunken centroid value obtained for a gene i from reference patients having a WHO grade 2 or grade 3 glioma, said reference patients having a median survival higher than 4 years.

According to the invention, the product P₂i is obtained from the following formula:
P₂i=Qi×V₂i, wherein V₂i corresponds to the shrunken centroid value obtained for a gene i from reference patients having a WHO grade 2 or grade 3 glioma, said reference patients having a median survival lower than 4 years.

The shrunken centroid value is established from data obtained from reference, or control, patients, belonging to a reference, or control, cohort of patients afflicted by either a WHO grade 2 or a WHO grade 3 glioma.

These reference, or control, patients are regrouped in a panel called cohort.

The cohort can be divided into two sub groups:
a subgroup of patient afflicted by WHO grade 2 glioma, or WHO grade 3 glioma, said patients having a median survival higher than four (4) years; said patients being considered as having a good prognosis of survival,
a subgroup of patient afflicted by WHO grade 2 glioma, or WHO grade 3 glioma, said patients having a median survival lower than four (4) years; said patients being considered as having a bad prognosis of survival.

From the entire cohort, it is possible to obtain the above subgroup by classifying patients according to a hierarchical clustering.

Cluster analysis or clustering is the assignment of a set of observations into subsets (called clusters) so that observations in the same cluster are similar in some sense.

Advantageously, the invention relates to the method as defined above, wherein said set comprise at least 7 genes belonging to said group of 22 genes, said at least 7 genes comprising or being constituted by the respective nucleic acid sequences SEQ ID NO: 1 to 7.

In one advantageous embodiment, the invention relates to the method as defined above, wherein said set comprise at least 9 genes belonging to said group of 22 genes, said at least said at least 9 genes comprising or being constituted by the respective nucleic acid sequences SEQ ID NO: 1 to 9.

Another advantageous embodiment of the invention relates to the method according to the previous definition, wherein said set consists of all the genes of said group of 22 genes

More advantageously, the invention relates to the method as defined above, wherein
if N1>N2, then said patient has a median survival higher than 4 years, preferably from 4 to 10 years, more preferably from 5 to 8 years, in particular about 6 years, and
if N1≦N2, then said patient has a median survival lower than 4 years, preferably from 0.5 to 3.5 years, more preferably from 0.5 to 2 years, in particular about 1 year,
   wherein

$$N 1 = {\sum }_{i = 1}^{n} ( {P}_{1} i ) - {T}_{1} = ( {\sum }_{i = 1}^{n} ( ( \frac{Qri - Qci}{Ji} ) \times {V}_{1} i ) ) - {T}_{1} ,$$

n varying from 3 to 22, and

$$N 2 = {\sum }_{i = 1}^{n} ( {P}_{2} i ) - {T}_{2} = ( {\sum }_{i = 1}^{n} ( ( \frac{Qri - Qci}{Ji} ) \times {V}_{2} i ) ) - {T}_{2} ,$$

n varying from 3 to 22,
wherein
Qri represents the quantitative raw expression value measured for a gene i in the biological sample of said subject, and
Qci represents the mean of the quantitative expression values obtained for said gene i from each patient of said control cohort of patient afflicted by a WHO grade 2 or grade 3 glioma,
Ji represents the standard deviation of the centroid values obtained for said gene i from each patient of said control cohort of patient afflicted by a WHO grade 2 or grade 3 glioma,
V₁i corresponds to the shrunken centroid value for said gene i obtained from control patients having a WHO grade 2 or grade 3 glioma with a median survival higher than 4 years,
V₂i corresponds to the shrunken centroid value for said gene i obtained from control patients having a WHO grade 2 or grade 3 glioma with a median survival lower than 4 years,
T1 corresponds to the training baseline value for control patients having a WHO grade 2 or grade 3 glioma with a median survival higher than 4 years, and
T2 corresponds to the training baseline value for control having a WHO grade 2 or grade 3 glioma with a median survival lower than 4 years.

Advantageously, the invention relates to a method as defined above, wherein the quantitative expression value Qi for a gene i is measured by quantitative techniques chosen among qRT-PCR and DNA Chip.

In one another advantageous embodiment, the invention relates to the method as defined above, wherein, when the quantitative technique is DNA CHIP, Qci values for a gene i are as follows:

| Genes | Qci |
|---|---|
| SEQ ID NO: 1 | 8.1111 |
| SEQ ID NO: 2 | 8.6287 |
| SEQ ID NO: 3 | 6.0748 |
| SEQ ID NO: 4 | 7.2020 |
| SEQ ID NO: 5 | 9.2810 |
| SEQ ID NO: 6 | 9.1734 |
| SEQ ID NO: 7 | 5.0310 |
| SEQ ID NO: 8 | 5.1660 |
| SEQ ID NO: 9 | 5.1174 |
| SEQ ID NO: 10 | 6.3898 |
| SEQ ID NO: 11 | 8.8992 |
| SEQ ID NO: 12 | 2.2380 |
| SEQ ID NO: 13 | 6.9486 |
| SEQ ID NO: 14 | 6.6286 |
| SEQ ID NO: 15 | 13.6886 |
| SEQ ID NO: 16 | 9.2036 |
| SEQ ID NO: 17 | 8.5740 |
| SEQ ID NO: 18 | 10.7286 |
| SEQ ID NO: 19 | 4.8529 |
| SEQ ID NO: 20 | 8.0629 |
| SEQ ID NO: 21 | 4.8347 |
| SEQ ID NO: 22 | 6.3091 |

In one another advantageous embodiment, the invention relates to the method as defined above, wherein, when the quantitative technique is qRT-PCR, Qci values for a gene i are as follows:

| Genes | Qci |
|---|---|
| SEQ ID NO: 1 | 9.8895 |
| SEQ ID NO: 2 | 10.7617 |
| SEQ ID NO: 3 | 4.8934 |
| SEQ ID NO: 4 | 8.6122 |
| SEQ ID NO: 5 | 10.0616 |
| SEQ ID NO: 6 | 9.1961 |
| SEQ ID NO: 7 | 7.0401 |
| SEQ ID NO: 8 | 6.7866 |
| SEQ ID NO: 9 | 7.4768 |
| SEQ ID NO: 10 | 8.4759 |
| SEQ ID NO: 11 | 8.4640 |
| SEQ ID NO: 12 | 5.5556 |
| SEQ ID NO: 13 | 9.2268 |
| SEQ ID NO: 14 | 7.4760 |
| SEQ ID NO: 15 | 16.4164 |
| SEQ ID NO: 16 | 7.4201 |
| SEQ ID NO: 17 | 11.9663 |
| SEQ ID NO: 18 | 11.3260 |
| SEQ ID NO: 19 | 9.2557 |
| SEQ ID NO: 20 | 8.4543 |
| SEQ ID NO: 21 | 6.9780 |
| SEQ ID NO: 22 | 7.2556 |

The invention also relates to a composition comprising oligonucleotides allowing the quantitative measure of the expression level of the genes of a set comprising at least 3 genes belonging to a group of 22 genes, said 22 genes comprising or being constituted by the respective nucleic acid sequences SEQ ID NO: 1 to 22,
wherein said at least 3 genes comprise or are constituted by the respective nucleic acid sequences SEQ ID NO: 1 to 3,
   preferably for its use for determining, preferably in vitro or ex vivo, from a biological sample of a subject afflicted by a WHO grade 2 or grade 3 glioma, the survival prognosis of said subject.

Advantageously, the invention relates to a composition as defined above, preferably for its use as defined above, wherein said set comprise at least 7 genes belonging to said group of 22 genes, said at least 7 genes comprising or being constituted by the respective nucleic acid sequences SEQ ID NO: 1 to 7.

Advantageously, the invention relates to a composition as defined above, preferably for its use as defined above, wherein said set comprise at least 9 genes belonging to a said group of 22 genes, said at least 9 genes comprising or being constituted by the respective nucleic acid sequences SEQ ID NO: 1 to 9.

Advantageously, the invention relates to a composition as defined above, preferably for its use as defined above, wherein said set consists of all the genes of said group of 22 genes.

Advantageously, the invention relates to a composition as defined above, preferably for its use as defined above, wherein said composition comprise at least a pair of oligonucleotides allowing the measure of the expression of the genes of said set of genes belonging to said group of 22 genes.

Advantageously, the invention relates to a composition as defined above, preferably for its use as defined above, wherein said composition comprises at least the oligonucleotides SEQ ID NO: 23-28, preferably at least the oligonucleotides SEQ ID NO: 23-40, more preferably at least the oligonucleotides SEQ ID NO: 23-42, more preferably at least the oligonucleotides SEQ ID NO: 23-54, chosen among the group consisting of the oligonucleotides SEQ ID NO: 23-66, and in particular said composition comprises the oligonucleotides SEQ ID NO: 23-66.

The invention also relates to a kit comprising:
oligonucleotides allowing the measure of the expression of the genes of a set comprising at least 3 genes belonging to a group of 22 genes, said 22 genes comprising or being constituted by the respective nucleic acid sequences SEQ ID NO: 1 to 22,
wherein said at least 3 genes comprise or are constituted by the respective nucleic acid sequences SEQ ID NO: 1 to 3, and
a support comprising data regarding the expression value of said at least 3 genes belonging to a group of 22 genes obtained from control patients.

The sequences SEQ ID NO: 1-22 corresponds to the genomic sequence of said genes.

Thus, as defined above, the invention propose to determine the expression of said genes, i.e. to determine the amount of the transcripts of said genes.

If a gene encodes more than 1 mRNA, they are called expression variants of said gene.

The preferred transcripts of the genes according to the invention are the following ones:
the gene CHI3L1 (SEQ ID NO: 1) expresses 5 variants: Variant 1 (Ensembl n^{o}ENST00000255409), Variant 2 (Ensembl n^{o}ENST00000404436), Variant 3 (Ensembl n^{o}ENST00000473185), Variant 4 (Ensembl n^{o}ENST00000472064) and Variant 5 (Ensembl n^{o}ENST00000478742),
the gene IGFBP2 (SEQ ID NO: 2) expresses 5 variants: Variant 1 (Ensembl n^{o}ENST00000233809), Variant 2 (Ensembl n^{o}ENST00000490362), Variant 3 (Ensembl n^{o}ENST00000434997), Variant 4 (Ensembl n^{o}ENST00000456764) and Variant 5 (Ensembl n^{o}ENST00000436812),
the gene POSTN (SEQ ID NO: 3) expresses 11 variants: Variant 1 (Ensembl n^{o}ENST00000379747), Variant 2 (Ensembl n^{o}ENST00000379742), Variant 3 (Ensembl n^{o}ENST00000379743), Variant 4 (Ensembl n^{o}ENST00000379749) and Variant 5 (Ensembl n^{o}ENST00000497145), Variant 6 (Ensembl n^{o}ENST00000478947), Variant 7 (Ensembl n^{o}ENST00000473823), Variant 8 (Ensembl n^{o}ENST00000474646), Variant 9 (Ensembl n^{o}ENST00000538347), Variant 10 (Ensembl n^{o}ENST00000541179) and Variant 11 (Ensembl n^{o}ENST00000541481),
the gene HSPG2 (SEQ ID NO: 4) express 16 variants: Variant 1 (Ensembl n^{o}ENST00000374695), Variant 2 (Ensembl n^{o}ENST00000486901), Variant 3 (Ensembl n^{o}ENST00000412328), Variant 4 (Ensembl n^{o}ENST00000374673) and Variant 5 (Ensembl n^{o}ENST00000439717), Variant 6 (Ensembl n^{o}ENST00000480900), Variant 7 (Ensembl n^{o}ENST00000498495), Variant 8 (Ensembl n^{o}ENST00000427897), Variant 9 (Ensembl n^{o}ENST00000493940), Variant 10 (Ensembl n^{o}ENST00000374676), Variant 11 (Ensembl n^{o}ENST00000469378), Variant 12 (Ensembl n^{o}ENST00000481644), Variant 13 (Ensembl n^{o}ENST00000426143), Variant 14 (Ensembl n^{o}ENST00000471322), Variant 15 (Ensembl n^{o}ENST00000453796) and Variant 16 (Ensembl n^{o}ENST00000430507),
the gene BMP2 (SEQ ID NO: 5) expresses only one mRNA (Ensembl n^{o}ENST00000378827),
the gene COL1A1 (SEQ ID NO: 6) expresses 13 variants: Variant 1 (Ensembl n^{o}ENST00000225964), Variant 2 (Ensembl n^{o}ENST00000474644), Variant 3 (Ensembl n^{o}ENST00000495677), Variant 4 (Ensembl n^{o}ENST00000485870) and Variant 5 (Ensembl n^{o}ENST00000463440), Variant 6 (Ensembl n^{o}ENST00000471344), Variant 7 (Ensembl n^{o}ENST00000476387), Variant 8 (Ensembl n^{o}ENST00000494334), Variant 9 (Ensembl n^{o}ENST00000486572), Variant 10 (Ensembl n^{o}ENST00000507689), Variant 11 (Ensembl n^{o}ENST00000504289), Variant 12 (Ensembl n^{o}ENST00000511732) and Variant 13 (Ensembl n^{o}ENST00000510710),
the gene NEK2 (SEQ ID NO: 7) expresses 5 variants: Variant 1 (Ensembl n^{o}ENST00000366999), Variant 2 (Ensembl n^{o}ENST00000366998), Variant 3 (Ensembl n^{o}ENST00000489633), Variant 4 (Ensembl n^{o}ENST00000462283) and Variant 5 (Ensembl n^{o}ENST00000540251),
the gene DLG7 (SEQ ID NO: 8) expresses 2 variants: Variant 1 (Ensembl n^{o}ENST00000247191) and Variant 2 (Ensembl n^{o}ENST00000395425),
the gene FOX M1 (SEQ ID NO: 9) expresses 9 variants: Variant 1 (Ensembl n^{o}ENST00000361953), Variant 2 (Ensembl n^{o}ENST00000359843), Variant 3 (Ensembl n^{o}ENST00000342628), Variant 4 (Ensembl n^{o}ENST00000536066) and Variant 5 (Ensembl n^{o}ENST00000538564), Variant 6 (Ensembl n^{o}ENST00000545049), Variant 7 (Ensembl n^{o}ENST00000366362), Variant 8 (Ensembl n^{o}ENST00000537018) and Variant 9 (Ensembl n^{o}ENST00000535350),
the gene BIRC5 (SEQ ID NO: 10) expresses 4 variants: Variant 1 (Ensembl n^{o}ENST00000301633), Variant 2 (Ensembl n^{o}ENST00000350051), Variant 3 (Ensembl n^{o}ENST00000374948) and Variant 4 (Ensembl n^{o}ENST00000432014),
the gene PLK1 (SEQ ID NO: 11) expresses 3 variants: Variant 1 (Ensembl n^{o}ENST00000300093), Variant 2 (Ensembl n^{o}ENST00000330792) and Variant 3 (Ensembl n^{o}ENST00000425844),
the gene NKX6-1 (SEQ ID NO: 12) expresses 2 variants: Variant 1 (Ensembl n^{o}ENST00000295886) and Variant 2 (Ensembl n^{o}ENST00000515820),
the gene NRG3(SEQ ID NO: 13) expresses 7 variants: Variant 1 (Ensembl n^{o}ENST00000372142), Variant 2 (Ensembl n^{o}ENST00000372141), Variant 3 (Ensembl n^{o}ENST00000404547), Variant 4 (Ensembl n^{o}ENST00000404576) and Variant 5 (Ensembl n^{o}ENST00000537287), Variant 6 (Ensembl n^{o}ENST00000537893), Variant 7 (Ensembl n^{o}ENST00000545131),
the gene BUB1B (SEQ ID NO: 14) expresses 3 variants: Variant 1 (Ensembl n^{o}ENST00000287598), Variant 2 (Ensembl n^{o}ENST00000412359) and Variant 3 (Ensembl n^{o}ENST00000442874),
the gene VIM (SEQ ID NO: 15) expresses 11 variants: Variant 1 (Ensembl n^{o}ENST00000224237), Variant 2 (Ensembl n^{o}ENST00000487938), Variant 3 (Ensembl n^{o}ENST00000469543), Variant 4 (Ensembl n^{o}ENST00000478317) and Variant 5 (Ensembl n^{o}ENST00000478746), Variant 6 (Ensembl n^{o}ENST00000497849), Variant 7 (Ensembl n^{o}ENST00000485947), Variant 8 (Ensembl n^{o}ENST00000421459), Variant 9 (Ensembl n^{o}ENST00000495528), Variant 10 (Ensembl n^{o}ENST00000544301) and Variant 11 (Ensembl n^{o}ENST00000545533),
the gene TNC (SEQ ID NO: 16) expresses 17 variants: Variant 1 (Ensembl n^{o}ENST00000350763), Variant 2 (Ensembl n^{o}ENST00000460345), Variant 3 (Ensembl n^{o}ENST00000476680), Variant 4 (Ensembl n^{o}ENST00000481475) and Variant 5 (Ensembl n^{o}ENST00000473855), Variant 6 (Ensembl n^{o}ENST00000498724), Variant 7 (Ensembl n^{o}ENST00000542877), Variant 8 (Ensembl n^{o}ENST00000423613), Variant 9 (Ensembl n^{o}ENST00000534839), Variant 10 (Ensembl n^{o}ENST00000341037), Variant 11 (Ensembl n^{o}ENST00000537320), Variant 12 (Ensembl n^{o}ENST00000544972), Variant 13 (Ensembl n^{o}ENST00000340094), Variant (Ensembl n^{o}ENST00000345230) and Variant 15 (Ensembl n^{o}ENST00000346706), Variant 16 (Ensembl n^{o}ENST00000442945) and Variant 17 (Ensembl n^{o}ENST00000535648),
the gene DLL3 (SEQ ID NO: 17) expresses 2 variants: Variant 1 (Ensembl n^{o}ENST00000205143), Variant 2 (Ensembl n^{o}ENST00000356433),
the gene JAG1 (SEQ ID NO: 18) expresses 3 variants: Variant 1 (Ensembl n^{o}ENST00000254958), Variant 2 (Ensembl n^{o}ENST00000488480) and Variant 3 (Ensembl n^{o}ENST00000423891),
the gene KI67 (SEQ ID NO: 19) expresses 8 variants: Variant 1 (Ensembl n^{o}ENST00000368654), Variant 2 (Ensembl n^{o}ENST00000368653), Variant 3 (Ensembl n^{o}ENST00000464771), Variant 4 (Ensembl n^{o}ENST00000478293) and Variant 5 (Ensembl n^{o}ENST00000484853), Variant 6 (Ensembl n^{o}ENST00000368652), Variant 7 (Ensembl n^{o}ENST00000537609) and Variant 8 (Ensembl n^{o}ENST00000538447),
the gene EZH2 (SEQ ID NO: 20) expresses 12 variants: Variant 1 (Ensembl n^{o}ENST00000483967), Variant 2 (Ensembl n^{o}ENST00000498186), Variant 3 (Ensembl n^{o}ENST00000492143), Variant 4 (Ensembl n^{o}ENST00000320356) and Variant 5 (Ensembl n^{o}ENST00000483012), Variant 6 (Ensembl n^{o}ENST00000478654), Variant 7 (Ensembl n^{o}ENST00000541220), Variant 8 (Ensembl n^{o}ENST00000460911), Variant 9 (Ensembl n^{o}ENST00000469631), Variant 10 (Ensembl n^{o}ENST00000350995), Variant 11 (Ensembl n^{o}ENST00000476773) and Variant 12 (Ensembl n^{o}ENST00000536783),
the gene BUB1 (SEQ ID NO: 21) expresses 13 variants: Variant 1 (Ensembl n^{o}ENST00000302759), Variant 2 (Ensembl n^{o}ENST00000409311), Variant 3 (Ensembl n^{o}ENST00000465029), Variant 4 (Ensembl n^{o}ENST00000466333) and Variant 5 (Ensembl n^{o}ENST00000420328), Variant 6 (Ensembl n^{o}ENST00000436916), Variant 7 (Ensembl n^{o}ENST00000447014), Variant 8 (Ensembl n^{o}ENST00000468927), Variant 9 (Ensembl n^{o}ENST00000477481), Variant 10 (Ensembl n^{o}ENST00000490632), Variant 11 (Ensembl n^{o}ENST00000478175), Variant 12 (Ensembl n^{o}ENST00000535254) and Variant 13 (Ensembl n^{o}ENST00000541432), and
the gene AURKA (SEQ ID NO: 22) expresses 14 variants: Variant 1 (Ensembl n^{o}ENST00000347343), Variant 2 (Ensembl n^{o}ENST00000441357), Variant 3 (Ensembl n^{o}ENST00000395915), Variant 4 (Ensembl n^{o}ENST00000395913) and Variant 5 (Ensembl n^{o}ENST00000456249), Variant 6 (Ensembl n^{o}ENST00000422322), Variant 7 (Ensembl n^{o}ENST00000420474), Variant 8 (Ensembl n^{o}ENST00000395914), Variant 9 (Ensembl n^{o}ENST00000395907), Variant 10 (Ensembl n^{o}ENST00000451915), Variant 11 (Ensembl n^{o}ENST00000312783), Variant 12 (Ensembl n^{o}ENST00000371356), Variant 13 (Ensembl n^{o}ENST00000395909), and Variant 13 (Ensembl n^{o}ENST00000395911).

The skilled person has sufficient guidance, referring to the Ensembl accession number, to determine what mRNA are quantified regarding a determined gene i.

For instance, the amount of the mRNA listed in the table 2 can be quantified according to the invention:

TABLE 2
represents the genes according to the invention, and their corresponding
SEQ ID, and, for each of said gene an example of mRNA represented
by its SEQ ID, and the corresponding Access number
in the NCBI database (http://www.ncbi.nlm.nih.gov/).

| | Gene | | |
|---|---|---|---|
| Gene SEQ ID | name | SEQ ID mRNA | SeqRef (of mRNA) |
| SEQ ID NO: 1 | CHI3L1 | SEQ ID NO: 67 | NM_001276 |
| SEQ ID NO: 2 | IGFBP2 | SEQ ID NO: 68 | NM_000597 |
| SEQ ID NO: 3 | POSTN | SEQ ID NO: 69 | NM_006475 |
| SEQ ID NO: 4 | HSPG2 | SEQ ID NO: 70 | NM_005529 |
| SEQ ID NO: 5 | BMP2 | SEQ ID NO: 71 | NM_001200 |
| SEQ ID NO: 6 | COL1A1 | SEQ ID NO: 72 | NM_000088 |
| SEQ ID NO: 7 | NEK2 | SEQ ID NO: 73 | NM_002497 |
| SEQ ID NO: 8 | DLG7 | SEQ ID NO: 74 | NM_014750 |
| SEQ ID NO: 9 | FOXM1 | SEQ ID NO: 75 | NM_021953 |
| SEQ ID NO: 10 | BIRC5 | SEQ ID NO: 76 | NM_001012270 |
| SEQ ID NO: 11 | PLK1 | SEQ ID NO: 77 | NM_005030 |
| SEQ ID NO: 12 | NKX6-1 | SEQ ID NO: 78 | NM_006168 |
| SEQ ID NO: 13 | NRG3 | SEQ ID NO: 79 | NM_001165972 |
| SEQ ID NO: 14 | BUB1B | SEQ ID NO: 80 | NM_001211 |
| SEQ ID NO: 15 | VIM | SEQ ID NO: 81 | NM_003380 |
| SEQ ID NO: 16 | TNC | SEQ ID NO: 82 | NM_002160 |
| SEQ ID NO: 17 | DLL3 | SEQ ID NO: 83 | NM_016941 |
| SEQ ID NO: 18 | JAG1 | SEQ ID NO: 84 | NM_000214 |
| SEQ ID NO: 19 | KI67 | SEQ ID NO: 85 | NM_002417 |
| SEQ ID NO: 20 | EZH2 | SEQ ID NO: 86 | NM_004456 |
| SEQ ID NO: 21 | BUB1 | SEQ ID NO: 87 | NM_004336 |
| SEQ ID NO: 22 | AURKA | SEQ ID NO: 88 | NM_003600 |

Thus, in the first step of the method according to the invention, the gene expression is measured by quantifying the amount of at least one variant listed above or at least one mRNA expressed by the genes according to the invention.

The invention also encompasses the mRNA having at least 90% identity with the above variants, which includes single-nucleotide polymorphism (SNP) or non phenotype associated mutations that can occur in DNA.

In one advantageous embodiment, the invention relates to the method as defined herein, wherein said set comprise at least 7 genes belonging to said group of 22 genes, said at least 7 genes comprising or being constituted by the respective nucleic acid sequences SEQ ID NO: 1 to 7.

Thus, according to this advantageous embodiment, the measure of the expression level of the genes represented by SEQ ID NO: 1, SEQ ID NO: 2, SEQ ID NO: 3, SEQ ID NO: 4, SEQ ID NO: 5, SEQ ID NO: 6 and SEQ ID NO: 7 is able to carry out the method according to the invention. In preferred embodiments this may yield a percentage of error of at most 5%.

Another advantageous embodiment of the invention relates to the method as defined above, wherein said set comprise at least 9 genes belonging to said group of 22 genes, said at least said at least 9 genes comprising or being constituted by the respective nucleic acid sequences SEQ ID NO: 1 to 9.

Thus, according to this advantageous embodiment, the measure of the expression level of the genes represented by SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 and SEQ ID NO: 9 is able to carry out the method according to the invention. In preferred embodiments this may yield a percentage of error of at most 5%.

The invention also relates to the method as defined above, wherein said set comprise at least 10 genes belonging to a said group of 22 genes, said at least 10 genes comprising or being constituted by the respective nucleic acid sequences SEQ ID NO: 1 to 10.

Thus, according to this advantageous embodiment, the measure of the expression level of the genes represented by SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 and SEQ ID NO: 10 is able to carry out the method according to the invention. In preferred embodiments this may yield a percentage of error of at most 5%.

The invention also relates to the method as defined above, wherein said set comprise at least 16 genes belonging to a said group of 22 genes, said at least 16 genes comprising or being constituted by the respective nucleic acid sequences SEQ ID NO: 1 to 16.

Thus, according to this advantageous embodiment, the measure of the expression level of the genes represented by SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15 and SEQ ID NO: 16 is able to carry out the method according to the invention. In preferred embodiments this may yield a percentage of error of at most 5%.

Thus in preferred embodiments the percentage of error according to the invention may be from 0 to 5%, preferably from 1 to 3%, more preferably from 0 to 1.5%.

A more advantageous embodiment of the invention relates to the method previously defined, wherein said set consists of all the genes of said group of 22 genes.

The lowest error rate is obtained when the expression level of all the 22 genes represented by the SEQ ID NO: 1-22 is measured.

### Sub-Group or Class Analysis

The expression of the genes, gene combinations, or gene signatures comprised above, when compared with a suitable reference (e.g. the outcome of the comparison in step (ii) above) is used to determine or predict a clinical phenotype. In particular the expression value described may be used to assign the sample to a class or “subgroup” of glioma patients having a particular predicted phenotype or prognosis.

It will be appreciated that from an entire cohort of patients, it is possible to define subgroups by classifying patients according to a hierarchical clustering.

Cluster analysis or clustering is the assignment of a set of observations into subsets (called clusters) so that observations in the same cluster are similar in some sense.

Hierarchical clustering is a commonly used statistical tool for exploring relationships in statistical data. It clusters data based on a user defined measure called “distance”. “Similarities”, “correlation”, are sometimes used in place of “distances”, because users' definition of “distance” is related to “similarities” or “correlation”. There are a large number of variants of hierarchical clustering. The differences are in the way distances are defined and computations (e.g., average-linkage, top-down) are implemented.

Preferably the cohort of glioma patients is divided into classes having the pre-defined survival prognosis. The expression value or signature is “compared with” a reference expression value or signature derived from each class in order to assign it to, or classify it as, one of the classes.

Preferably there are two classes, representing “good” or “bad” prognosis. The classes will be defined such as to ensure each contains a significant number of members of the cohort, but apart from this it will be understood that the classification may be done according to any desired prognosis criterion. The classifiers may be used to make a prediction in the absence of therapy, or to inform a decision about the requirement for therapy, or further therapy.

In one embodiment the desired prognosis criterion is survival period e.g. a median survival value of higher or lower than ‘Y’ years where Y may, for example, be 3 or 4 years. However the classes may be split according to other predefined risk factors established by post hoc analysis of the cohort of glioma patients.

### Assigning the Expression to a Class

A number of methods may be used to assign which class the sample is assigned to, or (to put it another way) to decide which “gene expression signature” the sample most closely matches.

At the simplest level, it will be appreciated that if the gene is routinely over-expressed in one group and under-expressed in the other, then whether or not the gene is over-expressed or under-expressed (e.g. based on the normalised, centred expression) can be used to assign it to one or other group.

Particularly where there are multiple genes, a linear combination or weighted average of the expression of the selected set of genes may be used to assign the sample to one or other group.

Example methods for defining and assigning the sample gene signature include those discussed by Diaz-Uriarte (2004) “Molecular Signatures from Gene Expression Data” available at http://www.citebase.org/abstract?id=oai:arXiv.org:q-bio/0401043 (see also supplementary material cited therein). Example methods for defining and assigning the sample gene signature include those discussed by Diaz-Uriarte (2004) “Molecular Signatures from Gene Expression Data” available at http://www.citebase.org/abstract? id=oai:arXiv.org:q-bio/0401043, like K nearest neighbors (KNN, therein and [1]) and support vector machines (therein and [2]). Example analyses non exhaustively include regression models (PLS [3], logistic regression [4]), linear discriminant analysis [5], weighted gene voting [6], centroid or shrunken centroid analysis [7], classification and regression trees [8] and machine learning methods like neural networks [9]. (1-Deegalla S, Boström H: Classification of microarrays with KNN: comparison of dimensionality reduction methods. Yin H et al. (Eds). IDEAL 2007, LNCS 4881, pp 800-809, 2007. http://people.dsv.su.se/^{˜}henke/papers/deegalla07.pdf; 2-Lee Y, Lee C K: Classification of multiple cancer types by multicategory support vector machines using gene expression data. Bioinformatics 2003, 19:1132-1139; 3-Gusnanto A, Pawitan Y, Ploner A: Variable selection in gene and protein expression data. Technical report, Department of Medical Epidemiology and Biostatistics, Karolinska Institutet, Stockholm, 2003; 4—Eilers P H C, Boer J M, van Ommen G J, van Houwelingen H C: Classification of microarray data with penalized logistic regression. Proceedings of SPIE volume 4266: progress in biomedical optics and imaging 2001, San José; 5-Dudoit S, Fridlyand J, Speed T P: Comparison of discrimination methods for the classification of tumors suing gene expression data. J Am Stat Assoc 2002, 97:77-87; 6-Ramaswamy S, Ross K N, Lander E S, Golub T R: A molecular signature of metastasis in primary solid tumors. Nature Genetics 2003, 33:49-54; 7-Tibshirani R, Hastie T, Narasimhan B, Chu G: Diagnosis of multiple cancer types by shrunken centroids of gene expression. Proc Natl Acad Sci USA 2002, 99:6567-6572; 8-Peter J. Tan, David L. Dowe, Trevor I. Dix: Building Classification Models from Microarray Data with Tree-Based Classification Algorithms. Australian Conference on Artificial Intelligence 2007: 589-598; 9—O'Neill M C, Song L: Neural network analysis of lymphoma microarray data: prognosis and diagnosis near-perfect. BMC Bioinformatics 2003, 4:13)

### Preferred Statistical Analysis—Use of Centroids

A preferred method for use in the present invention is shrunken centroid analysis, which is described in more detail hereinafter. It will be appreciated that this could be performed mutatis mutandis based on centroids rather than shrunken centroids.

In this embodiment the invention relates to a method for determining, preferably in vitro or ex vivo, from a biological sample of a subject afflicted by a WHO grade 2 or grade 3 glioma, the survival prognosis of said patient,

### Said Method Comprising:

determining the quantitative expression value Qi for each gene of a set which preferably comprises at least X genes belonging to a group of 22 genes, said 22 genes comprising to or being constituted by the respective nucleic acid sequences SEQ ID NO: 1 to 22,
establishing
   a first product P₁i for each of said at least X genes, between the respective Qi values obtained above for each said at least X genes and a first value V₁i, and
   a second product P₂i for each of said at least X genes, between the respective Qi values obtained above for each said at least X genes and a second value V₂i,
wherein
   said first value Vii corresponds to the shrunken centroid value for a gene i obtained from reference patients having a WHO grade 2 or grade 3 glioma, said reference patients having a median survival higher than Y years, and
   said second value V2i corresponds to the shrunken centroid value for a gene i obtained from reference patients having a WHO grade 2 or grade 3 glioma, said reference patients having a median survival lower than Y years,
      said patients having a WHO grade 2 or grade 3 glioma with a median survival lower or higher than Y years belonging to a reference cohort of patients afflicted by either a WHO grade 2 or a WHO grade 3 glioma,
determining the survival rate of said patient as follows:
   if the sum of the P₁i products of each of said at least X genes is higher than the sum of the P₂i products of each of said at least X genes, then said subject has a median survival higher than Y years, and
   if the sum of the P₁i products of each of said at least X genes is lower than or equal to the sum of the P₂i products of each of said at least X genes, then said subject has a median survival lower than Y years.

Preferably ‘Y’ years is simply an illustrative pre-determined clinically relevant survival rate. Typically it may be 4 i.e. the method can be used to stratify patients into groups of subjects having predicted survival rates of higher or lower than 4 years.

Preferably X is 3 i.e. the expression of at least 3 genes are assessed. The present Inventors have shown that the expression level of at least 3 determined genes belonging to a group of 22 determined genes is sufficient to propose an effective prognosis method of individuals afflicted by gliomas,

Said least 3 determined genes being preferably: CHI3L1, IGFBP2 and POSTN. i.e. the 3 genes preferably comprise or are constituted by the respective nucleic acid sequences SEQ ID NO: 1 to 3.

As a part of the method according to this embodiment of the invention, two products (mathematical products) are calculated for each gene i, i.e. for each gene of said at least 3 genes belonging to the group of 22 genes:

P₁i: the first product P₁ for a determined gene i (e.g. SEQ ID NO: i, i varying from 1 to at least 3), and

P₂i: the second product P₂ for a determined gene i (e.g. SEQ ID NO: i, i varying from 1 to at least 3).

As mentioned above, regarding the definition of the i variable, the first product P₁ for the gene SEQ ID NO: 1 will be annotated P₁1, the first product P₁ for the gene SEQ ID NO: 2 will be annotated P₁2, first product P₁ for the gene SEQ ID NO: 3 will be annotated P₁3, etc. . . .

In the same way, the second product P₂ for the gene SEQ ID NO: 1 will be annotated P₂1, the second product P₂ for the gene SEQ ID NO: 2 will be annotated P₁2, first product P₂ for the gene SEQ ID NO: 3 will be annotated P₂3, etc. . . .

According to the invention, the product P₁i is obtained from the following formula: P₁i=Qi×V₁i, wherein V₁i corresponds to the shrunken centroid value obtained for a gene i from reference patients having a WHO grade 2 or grade 3 glioma, said reference patients having a median survival higher than Y (e.g. 4) years.

According to the invention, the product P₂i is obtained from the following formula: P₂i=Qi×V₂i, wherein V₂i corresponds to the shrunken centroid value obtained for a gene i from reference patients having a WHO grade 2 or grade 3 glioma, said reference patients having a median survival lower than Y (e.g. 4) years.

The shrunken centroid value is established from data obtained from reference, or control, patients, belonging to a reference, or control, cohort of patients afflicted by either a WHO grade 2 or a WHO grade 3 glioma.

As noted above, reference, or control, patients are regrouped in a panel called cohort. The cohort can be divided into two sub groups:
a subgroup of patient afflicted by WHO grade 2 glioma, or WHO grade 3 glioma, said patients having a median survival higher than Y (e.g. 4) years; said patients being considered as having a good prognosis of survival,
a subgroup of patient afflicted by WHO grade 2 glioma, or WHO grade 3 glioma, said patients having a median survival lower than Y (e.g. (4) years; said patients being considered as having a bad prognosis of survival.

From the data of the reference patients belonging to the cohort, it is possible, to determine a shrunken centroid value from the quantitative value Qi obtained for each gene i of at least the 3 genes e.g. SEQ ID NO: 1, SEQ ID NO: 2 and SEQ ID NO: 3.

The shrunken centroid calculation is well known in the art, and disclosed for instance in Narashiman and Chu, [Narashiman and Chu (2002) PNAS 99:6567-6572]

The centroid is the average gene expression for each gene in each class divided by the within-class standard deviation for that gene.

Nearest centroid classification takes the gene expression profile of a new sample, and compares it to each of these class centroids. The class whose centroid that it is closest to, in distance, is the predicted class for that new sample.

Nearest shrunken centroid classification makes one important modification to standard nearest centroid classification. It “shrinks” each of the class centroids toward the overall centroid for all classes by an amount we call the threshold. This shrinkage consists of moving the centroid towards zero by threshold, setting it equal to zero if it hits zero. For example if threshold was 2.0, a centroid of 3.2 would be shrunk to 1.2, a centroid of −3.4 would be shrunk to −1.4, and a centroid of 1.2 would be shrunk to zero.

After shrinking the centroids, the new sample is classified by the usual nearest centroid rule, but using the shrunken class centroids.

This shrinkage has two advantages:

1) it can make the classifier more accurate by reducing the effect of noisy genes,

2) it does automatic gene selection.

In particular, if a gene is shrunk to zero for all classes, then it is eliminated from the prediction rule. Alternatively, it may be set to zero for all classes except one, and we learn that high or low expression for that gene characterizes that class.

The user decides on the value to use for threshold. Typically one examines a number of different choices.

From the patients of the first subgroup, a shrunken centroid V₁ value is determined for each gene, e.g. for each of the genes of said at least 3 genes of SEQ ID NO: 1, SEQ ID NO: 2 and SEQ ID NO: 3 belonging to the group of 22 genes.

From the patients of the second subgroup, a shrunken centroid V₂ value is determined for each gene, e.g. for each of the genes of said at least 3 genes of SEQ ID NO: 1, SEQ ID NO: 2 and SEQ ID NO: 3 belonging to the group of 22 genes.

In other words, for a determined gene i, two shrunken centroid values are obtained.

By way of example, if only the expression value of said at least 3 genes (SEQ ID NO: 1-3) is considered, 6 shrunken centroid values will be used:
V₁1 and V₂1, for the gene SEQ ID NO: 1
V₁2 and V₂2, for the gene SEQ ID NO: 2, and
V₁3 and V₂3, for the gene SEQ ID NO: 3.

Also, at the end of the step 2 of the method according to the invention, if only the expression value of said at least 3 genes (SEQ ID NO: 1-3) is considered, 6 products P will be obtained:
P₁1 and P₂1, for the gene SEQ ID NO: 1
P₁2 and P₂2, for the gene SEQ ID NO: 2, and
P₁3 and P₂3, for the gene SEQ ID NO: 3.

The third step of this embodiment of a method according to the invention corresponds to the comparison of the sum of the products P obtained at the previous step “corrected” by subtracting the training baseline T to each of the sums, i.e. T₁ and T₂.

The training baseline represents the “position” of the centroids in the space of the genes used to build the predictor.

### According to the Invention:

T1 corresponds to the baseline value for control patients having a WHO grade 2 or grade 3 glioma with a median survival higher than 4 years, and
T2 corresponds to the baseline value for control having a WHO grade 2 or grade 3 glioma with a median survival lower than 4 years.

Thus, if the sum of the P₁ product minus the baseline is higher than the sum of the P₂ product minus the baseline, therefore, the biological of the patient from which the expression levels of said at least (say) 3 genes have been calculated corresponds to a low grade glioma, with a good prognosis of survival, and the patient have a median of survival higher than (say) 4 years.

On the contrary, if the sum of the P₁ product minus the baseline is lower than, or equal to, the sum of the P₂ product minus the baseline, therefore, the biological of the patient from which the expression levels of said at least (say) 3 genes have been calculated corresponds to a low grade glioma, with a bad prognosis of survival, and the patient have a median of survival lower than (say) 4 years.

For instance, in the case of only the expression level of the genes SEQ ID NO: 1, SEQ NO: 2 and SEQ ID NO: 3 is measured, the prognosis conclusion will be as follows:

$$\begin{matrix}if ( {\sum }_{i = 1}^{3} {P}_{1} i ) - {T}_{1} = ( {P}_{1} 1 + {P}_{1} 2 + {P}_{1} 3 ) - {T}_{1} > \\ ( {\sum }_{i = 1}^{3} {P}_{2} i ) - {T}_{2} \\ = ( {P}_{2} 1 + {P}_{2} 2 + {P}_{2} 3 ) - {T}_{2} ,\end{matrix}$$

then the patient have a good prognosis of survival, and has a median survival higher than 4 years, and

$$\begin{matrix}if ( {\sum }_{i = 1}^{3} {P}_{1} i ) - {T}_{1} = ( {P}_{1} 1 + {P}_{1} 2 + {P}_{1} 3 ) - {T}_{1} \leq \\ ( {\sum }_{i = 1}^{3} {P}_{2} i ) - {T}_{2} \\ = ( {P}_{2} 1 + {P}_{2} 2 + {P}_{2} 3 ) - {T}_{2} ,\end{matrix}$$

then the patient have a bad prognosis of survival, and has a median survival lower than 4 years.

The same applies mutatis mutandis for 4 to 22 genes of the group of 22 genes according to the invention.

To summarize, in one embodiment according to the invention is as follows:

In a biological sample of a patient afflicted by a low grade glioma:
1—the expression level of at least the genes of SEQ ID NO: 1, SEQ ID NO: 2 and SEQ ID NO: 3, among a group of 22 genes represented by the respective sequences SEQ ID NO: 1-22, is measured, to obtain a quantitative value Qi for each of said at least 3 genes,
2—For each of said at least 3 genes the products P₁i and P₂i is determined such that
   P₁i=Qi×V₁i, wherein V₁i is the shrunken centroid value for a gene i obtained from reference patients having a low grade glioma, said patient having a median survival higher than 4 years, and
   P₂i=Qi×V₂i, wherein V₂i is the shrunken centroid value for a gene i obtained from reference patients having a low grade glioma, said patient having a median survival lower than 4 years.
3—For each of said at least 3 genes, the sum of P₁i and P₂i products is established, and
   if the sum of P₁i>sum of P₂i, then the patient have a good prognosis (median survival>4 years),
   if the sum of P₁i≦sum of P₂i, then the patient have a good prognosis (median survival<4 years),
preferably
   if the sum of P₁i−T₁>sum of P₂i−T₂, then the patient have a good prognosis (median survival>4 years),
   if the sum of P₁i−T₁≦sum of P₂i−T₂, then the patient have a good prognosis (median survival<4 years),

The invention also relates to a method as defined above, wherein the quantitative expression value Qi for a gene i corresponds to the comparison between:
the quantitative raw expression value Qri measured for a gene i, in the biological sample of said subject, and
a Qci value corresponding to the mean of the quantitative expression values obtained for said gene i from each patient of said control cohort of patient afflicted by a WHO grade 2 or grade 3 glioma,
   the Qi value being such that Qi=Qri−Qci.

As explained previously, preferably according to the invention, the quantitative raw expression value Qri is a normalized value of the signal detected for a gene i.

In still another advantageous embodiment, the invention relates to the method previously defined, wherein
if N1>N2, then said patient has a median survival higher than Y years, preferably higher than 4 years, preferably from 4 to 10 years, more preferably from 5 to 8 years, in particular about 6 years, and
if N1≦N2, then said patient has a median survival lower than Y years, preferably lower than 4 years, preferably from 0.5 to 3.5 years, more preferably from 0.5 to 2 years, in particular about 1 year,
   wherein

$$N 1 = {\sum }_{i = 1}^{n} ( {P}_{1} i ) - {T}_{1} = ( {\sum }_{i = 1}^{n} ( ( \frac{Qri - Qci}{Ji} ) \times {V}_{1} i ) ) - {T}_{1} ,$$

n varying from 3 to 22, and

$$N 2 = {\sum }_{i = 1}^{n} ( {P}_{2} i ) - {T}_{2} = ( {\sum }_{i = 1}^{n} ( ( \frac{Qri - Qci}{Ji} ) \times {V}_{2} i ) ) - {T}_{2} ,$$

n varying from 3 to 22,
wherein
Qri represents the quantitative raw expression value measured for a gene i in the biological sample of said subject, and
Qci represents the mean of the quantitative expression values obtained for said gene i from each patient of said control cohort of patient afflicted by a WHO grade 2 or grade 3 glioma,
Ji represents the standard deviation of the shrunken centroid values obtained for said gene i from each patient of said control cohort of patient afflicted by a WHO grade 2 or grade 3 glioma,
V₁i corresponds to the shrunken centroïd value for said gene i obtained from control patients having a WHO grade 2 or grade 3 glioma with a median survival higher than Y years,
V₂i corresponds to the shrunken centroïd value for said gene i obtained from control patients having a WHO grade 2 or grade 3 glioma with a median survival lower than Y years,
T1 corresponds to the baseline value for control patients having a WHO grade 2 or grade 3 glioma with a median survival higher than Y years, and
T2 corresponds to the baseline value for control having a WHO grade 2 or grade 3 glioma with a median survival lower than Y years.

According to the invention, the formula disclosed above can be expressed as follows, when Qri is measured by PCR:

$$N 1 = {\sum }_{i = 1}^{n} ( {P}_{1} i ) - {T}_{1} = ( {\sum }_{i = 1}^{n} ( ( \frac{\begin{matrix}{log}_{2} ( \frac{Si}{Sc} \times 1000 ) - \frac{1}{size ( training )} \times \\ {\sum }_{training} {log}_{2} ( \frac{Si ( training )}{Sc} \times 1000 )\end{matrix}}{Ji} ) \times {V}_{1} i ) ) - {T}_{1} ,$$

n which will preferably vary from 3 to 22, and

$$N 2 = {\sum }_{i = 1}^{n} ( {P}_{2} i ) - {T}_{2} = ( {\sum }_{i = 1}^{n} ( ( \frac{\begin{matrix}{log}_{2} ( \frac{Si}{Sc} \times 1000 ) - \frac{1}{size ( training )} \times \\ {\sum }_{training} {log}_{2} ( \frac{Si ( training )}{Sc} \times 1000 )\end{matrix}}{Ji} ) \times {V}_{2} i ) ) - T 2 , n$$

which will preferably vary from 3 to 22,
wherein
Qri represents the quantitative raw expression value measured for a gene i in the biological sample of said subject, and
Qci represents the mean of the quantitative expression values obtained for said gene i from each patient of said control cohort of patient afflicted by a WHO grade 2 or grade 3 glioma,
Ji represents the standard deviation of the shrunken centroid values obtained for said gene i from each patient of said control cohort of patient afflicted by a WHO grade 2 or grade 3 glioma,
V₁i corresponds to the shrunken centroïd value for said gene i obtained from control patients having a WHO grade 2 or grade 3 glioma with a median survival higher than Y years,
V₂i corresponds to the shrunken centroïd value for said gene i obtained from control patients having a WHO grade 2 or grade 3 glioma with a median survival lower than Y years,
T₁ corresponds to the training baseline value for control patients having a WHO grade 2 or grade 3 glioma with a median survival higher than Y years, and
T₂ corresponds to the training baseline value for control having a WHO grade 2 or grade 3 glioma with a median survival lower than Y years.

In still another embodiment, the invention relates to the method as defined above, wherein, when the quantitative technique is qRT-PCR, Qci values for a gene i are as follows:

| Genes | Qci |
|---|---|
| SEQ ID NO: 1 | 9.8895 |
| SEQ ID NO: 2 | 10.7617 |
| SEQ ID NO: 3 | 4.8934 |
| SEQ ID NO: 4 | 8.6122 |
| SEQ ID NO: 5 | 10.0616 |
| SEQ ID NO: 6 | 9.1961 |
| SEQ ID NO: 7 | 7.0401 |
| SEQ ID NO: 8 | 6.7866 |
| SEQ ID NO: 9 | 7.4768 |
| SEQ ID NO: 10 | 8.4759 |
| SEQ ID NO: 11 | 8.4640 |
| SEQ ID NO: 12 | 5.5556 |
| SEQ ID NO: 13 | 9.2268 |
| SEQ ID NO: 14 | 7.4760 |
| SEQ ID NO: 15 | 16.4164 |
| SEQ ID NO: 16 | 7.4201 |
| SEQ ID NO: 17 | 11.9663 |
| SEQ ID NO: 18 | 11.3260 |
| SEQ ID NO: 19 | 9.2557 |
| SEQ ID NO: 20 | 8.4543 |
| SEQ ID NO: 21 | 6.9780 |
| SEQ ID NO: 22 | 7.2556 |

In one advantageous embodiment, the invention relates to the method as defined above, wherein, when the quantitative technique is qRT-PCR, Qci, Ji, V₁i, V₂i, T1 and T2 are as follows:
when the expression level of the genes SEQ ID NO: 1-3 is measured

| 3 genes | Qci | Ji | V1i | V2i | T1 | T2 |
|---|---|---|---|---|---|---|
| SEQ ID NO: 1 | 9.8895 | 3.5040 | −0.26557206 | 0.5975371 | 0.421766 | 1.4522384 |
| SEQ ID NO: 2 | 10.7617 | 2.8662 | −0.18905578 | 0.4253755 | | |
| SEQ ID NO: 3 | 4.8934 | 4.6331 | −0.04256449 | 0.0957701 | | |

when the expression level of the genes SEQ ID NO: 1-7 is measured

| 7 genes | Qci | Ji | V1i | V2i | T1 | T2 |
|---|---|---|---|---|---|---|
| SEQ ID NO: 1 | 9.8895 | 3.5040 | −0.309811118 | 0.697075015 | 0.4468138 | 1.5790433 |
| SEQ ID NO: 2 | 10.7617 | 2.8662 | −0.233294833 | 0.524913374 | | |
| SEQ ID NO: 3 | 4.8934 | 4.6331 | −0.086803548 | 0.195307982 | | |
| SEQ ID NO: 4 | 8.6122 | 2.5811 | −0.011870396 | 0.026708392 | | |
| SEQ ID NO: 5 | 10.0616 | 2.5943 | 0.008475628 | −0.019070162 | | |
| SEQ ID NO: 6 | 9.1961 | 3.4356 | −0.003268925 | 0.007355082 | | |
| SEQ ID NO: 7 | 7.0401 | 2.5542 | −0.003223563 | 0.007253016 | | |

when the expression level of the genes SEQ ID NO: 1-9 is measured

| 9 genes | Qci | Ji | V1i | V2i | T1 | T2 |
|---|---|---|---|---|---|---|
| SEQ ID NO: 1 | 9.8895 | 3.5040 | −0.331889301 | 0.746750927 | 0.4631175 | 1.6615805 |
| SEQ ID NO: 2 | 10.7617 | 2.8662 | −0.255373016 | 0.574589285 | | |
| SEQ ID NO: 3 | 4.8934 | 4.6331 | −0.10888173 | 0.244983893 | | |
| SEQ ID NO: 4 | 8.6122 | 2.5811 | −0.033948579 | 0.076384303 | | |
| SEQ ID NO: 5 | 10.0616 | 2.5943 | 0.03055381 | −0.068746073 | | |
| SEQ ID NO: 6 | 9.1961 | 3.4356 | −0.025347108 | 0.057030993 | | |
| SEQ ID NO: 7 | 7.0401 | 2.5542 | −0.025301745 | 0.056928927 | | |
| SEQ ID NO: 8 | 6.7866 | 3.1202 | −0.013802309 | 0.031055196 | | |
| SEQ ID NO: 9 | 7.4768 | 2.7594 | −0.002251371 | 0.005065584 | | |

when the expression level of the genes SEQ ID NO: 1-10 is measured

| 10 genes | Qci | Ji | V1i | V2i | T1 | T2 |
|---|---|---|---|---|---|---|
| SEQ ID NO: 1 | 9.8895 | 3.5040 | −0.37621105 | 0.84647485 | 0.509496 | 1.896372 |
| SEQ ID NO: 2 | 10.7617 | 2.8662 | −0.29969476 | 0.67431321 | | |
| SEQ ID NO: 3 | 4.8934 | 4.6331 | −0.15320348 | 0.34470782 | | |
| SEQ ID NO: 4 | 8.6122 | 2.5811 | −0.07827032 | 0.17610823 | | |
| SEQ ID NO: 5 | 10.0616 | 2.5943 | 0.07487556 | −0.16847 | | |
| SEQ ID NO: 6 | 9.1961 | 3.4356 | −0.06966885 | 0.15675492 | | |
| SEQ ID NO: 7 | 7.0401 | 2.5542 | −0.06962349 | 0.15665285 | | |
| SEQ ID NO: 8 | 6.7866 | 3.1202 | −0.05812405 | 0.13077912 | | |
| SEQ ID NO: 9 | 7.4768 | 2.7594 | −0.04657312 | 0.10478951 | | |
| SEQ ID NO: 10 | 8.4759 | 2.9469 | −0.04169181 | 0.09380658 | | |

when the expression level of the genes SEQ ID NO: 1-16 is measured

| 16 genes | Qci | Ji | V1i | V2i | T1 | T2 |
|---|---|---|---|---|---|---|
| SEQ ID NO: 1 | 9.8895 | 3.5040 | −0.398289229 | 0.896150764 | 0.540277 | 2.052201 |
| SEQ ID NO: 2 | 10.7617 | 2.8662 | −0.321772944 | 0.723989123 | | |
| SEQ ID NO: 3 | 4.8934 | 4.6331 | −0.175281658 | 0.394383731 | | |
| SEQ ID NO: 4 | 8.6122 | 2.5811 | −0.100348507 | 0.225784141 | | |
| SEQ ID NO: 5 | 10.0616 | 2.5943 | 0.096953738 | −0.218145911 | | |
| SEQ ID NO: 6 | 9.1961 | 3.4356 | −0.091747036 | 0.206430831 | | |
| SEQ ID NO: 7 | 7.0401 | 2.5542 | −0.091701673 | 0.206328765 | | |
| SEQ ID NO: 8 | 6.7866 | 3.1202 | −0.080202237 | 0.180455034 | | |
| SEQ ID NO: 9 | 7.4768 | 2.7594 | −0.068651299 | 0.154465422 | | |
| SEQ ID NO: 10 | 8.4759 | 2.9469 | −0.063769996 | 0.143482491 | | |
| SEQ ID NO: 11 | 8.4640 | 2.1597 | −0.020277623 | 0.045624651 | | |
| SEQ ID NO: 12 | 5.5556 | 2.3964 | −0.01079938 | 0.024298604 | | |
| SEQ ID NO: 13 | 9.2268 | 3.1865 | 0.008786792 | −0.019770281 | | |
| SEQ ID NO: 14 | 7.4760 | 2.6144 | −0.006607988 | 0.014867974 | | |
| SEQ ID NO: 15 | 16.4164 | 2.8714 | −0.006204653 | 0.013960469 | | |
| SEQ ID NO: 16 | 7.4201 | 3.3385 | −0.003597575 | 0.008094544 | | |

when the expression level of the genes SEQ ID NO: 1-22 is measured

| 22 genes | Qci | Ji | V1i | V2i | T1 | T2 |
|---|---|---|---|---|---|---|
| SEQ ID NO: 1 | 9.8895 | 3.5040 | −0.442610974 | 0.995874691 | 0.6255484 | 2.4838871 |
| SEQ ID NO: 2 | 10.7617 | 2.8662 | −0.366094689 | 0.82371305 | | |
| SEQ ID NO: 3 | 4.8934 | 4.6331 | −0.219603403 | 0.494107658 | | |
| SEQ ID NO: 4 | 8.6122 | 2.5811 | −0.144670252 | 0.325508068 | | |
| SEQ ID NO: 5 | 10.0616 | 2.5943 | 0.141275483 | −0.317869838 | | |
| SEQ ID NO: 6 | 9.1961 | 3.4356 | −0.136068781 | 0.306154758 | | |
| SEQ ID NO: 7 | 7.0401 | 2.5542 | −0.136023419 | 0.306052692 | | |
| SEQ ID NO: 8 | 6.7866 | 3.1202 | −0.124523982 | 0.28017896 | | |
| SEQ ID NO: 9 | 7.4768 | 2.7594 | −0.112973044 | 0.254189348 | | |
| SEQ ID NO: 10 | 8.4759 | 2.9469 | −0.108091741 | 0.243206417 | | |
| SEQ ID NO: 11 | 8.4640 | 2.1597 | −0.064599368 | 0.145348578 | | |
| SEQ ID NO: 12 | 5.5556 | 2.3964 | −0.055121125 | 0.124022531 | | |
| SEQ ID NO: 13 | 9.2268 | 3.1865 | 0.053108537 | −0.119494208 | | |
| SEQ ID NO: 14 | 7.4760 | 2.6144 | −0.050929734 | 0.114591901 | | |
| SEQ ID NO: 15 | 16.4164 | 2.8714 | −0.050526398 | 0.113684396 | | |
| SEQ ID NO: 16 | 7.4201 | 3.3385 | −0.04791932 | 0.107818471 | | |
| SEQ ID NO: 17 | 11.9663 | 3.4954 | 0.030451917 | −0.068516814 | | |
| SEQ ID NO: 18 | 11.3260 | 2.2250 | −0.029802867 | 0.067056452 | | |
| SEQ ID NO: 19 | 9.2557 | 3.1583 | −0.014836187 | 0.033381421 | | |
| SEQ ID NO: 20 | 8.4543 | 2.5087 | −0.010433641 | 0.023475692 | | |
| SEQ ID NO: 21 | 6.9780 | 4.4847 | −0.002903001 | 0.006531752 | | |
| SEQ ID NO: 22 | 7.2556 | 2.6921 | −0.002374696 | 0.005343066 | | |

The above matrices are appropriate to carry out the method according to the invention, when the prognosis of a patient, for which the expression level of said at least 3 genes according to the invention has been quantified by qRT-PCR, is evaluated.

The above values correspond to the values obtained for a determined cohort of reference patients having a WHO grade 2 or grade 3 glioma.

Applying the method disclosed in the Example, the skilled person could easily obtain similar results from any other determined cohort.

In still another embodiment, the invention relates to the method as defined above, wherein, when the quantitative technique is DNA CHIP, Qci values for a gene i are as follows:

| Genes | Qci |
|---|---|
| SEQ ID NO: 1 | 8.1111 |
| SEQ ID NO: 2 | 8.6287 |
| SEQ ID NO: 3 | 6.0748 |
| SEQ ID NO: 4 | 7.2020 |
| SEQ ID NO: 5 | 9.2810 |
| SEQ ID NO: 6 | 9.1734 |
| SEQ ID NO: 7 | 5.0310 |
| SEQ ID NO: 8 | 5.1660 |
| SEQ ID NO: 9 | 5.1174 |
| SEQ ID NO: 10 | 6.3898 |
| SEQ ID NO: 11 | 8.8992 |
| SEQ ID NO: 12 | 2.2380 |
| SEQ ID NO: 13 | 6.9486 |
| SEQ ID NO: 14 | 6.6286 |
| SEQ ID NO: 15 | 13.6886 |
| SEQ ID NO: 16 | 9.2036 |
| SEQ ID NO: 17 | 8.5740 |
| SEQ ID NO: 18 | 10.7286 |
| SEQ ID NO: 19 | 4.8529 |
| SEQ ID NO: 20 | 8.0629 |
| SEQ ID NO: 21 | 4.8347 |
| SEQ ID NO: 22 | 6.3091 |

In still another embodiment, the invention relates to the method as defined above, wherein, when the quantitative technique is DNA CHIP, Qci, Ji, V₁i, V₂i, T1 and T2 are as follows:
when the expression level of the genes SEQ ID NO: 1-3 is measured

| 3 genes | Qci | Ji | V1i | V2i | T1 | T2 |
|---|---|---|---|---|---|---|
| SEQ ID NO: 1 | 8.1111 | 3.5040 | −0.26557206 | 0.5975371 | 0.421766 | 1.4522384 |
| SEQ ID NO: 2 | 8.6287 | 2.8662 | −0.18905578 | 0.4253755 | | |
| SEQ ID NO: 3 | 6.0748 | 4.6331 | −0.04256449 | 0.0957701 | | |

when the expression level of the genes SEQ ID NO: 1-7 is measured

| 7 genes | Qci | Ji | V1i | V2i | T1 | T2 |
|---|---|---|---|---|---|---|
| SEQ ID NO: 1 | 8.1111 | 3.5040 | −0.309811118 | 0.697075015 | 0.4468138 | 1.5790433 |
| SEQ ID NO: 2 | 8.6287 | 2.8662 | −0.233294833 | 0.524913374 | | |
| SEQ ID NO: 3 | 6.0748 | 4.6331 | −0.086803548 | 0.195307982 | | |
| SEQ ID NO: 4 | 7.2020 | 2.5811 | −0.011870396 | 0.026708392 | | |
| SEQ ID NO: 5 | 9.2810 | 2.5943 | 0.008475628 | −0.019070162 | | |
| SEQ ID NO: 6 | 9.1734 | 3.4356 | −0.003268925 | 0.007355082 | | |
| SEQ ID NO: 7 | 5.0310 | 2.5542 | −0.003223563 | 0.007253016 | | |

| 9 genes | Qci | Ji | V1i | V2i | T1 | T2 |
|---|---|---|---|---|---|---|
| SEQ ID NO: 1 | 8.1111 | 3.5040 | −0.331889301 | 0.746750927 | 0.4631175 | 1.6615805 |
| SEQ ID NO: 2 | 8.6287 | 2.8662 | −0.255373016 | 0.574589285 | | |
| SEQ ID NO: 3 | 6.0748 | 4.6331 | −0.10888173 | 0.244983893 | | |
| SEQ ID NO: 4 | 7.2020 | 2.5811 | −0.033948579 | 0.076384303 | | |
| SEQ ID NO: 5 | 9.2810 | 2.5943 | 0.03055381 | −0.068746073 | | |
| SEQ ID NO: 6 | 9.1734 | 3.4356 | −0.025347108 | 0.057030993 | | |
| SEQ ID NO: 7 | 5.0310 | 2.5542 | −0.025301745 | 0.056928927 | | |
| SEQ ID NO: 8 | 5.1660 | 3.1202 | −0.013802309 | 0.031055196 | | |
| SEQ ID NO: 9 | 5.1174 | 2.7594 | −0.002251371 | 0.005065584 | | |

when the expression level of the genes SEQ ID NO: 1-9 is measured

| 10 genes | Qci | Ji | V1i | V2i | T1 | T2 |
|---|---|---|---|---|---|---|
| SEQ ID NO: 1 | 8.1111 | 3.5040 | −0.37621105 | 0.84647485 | 0.509496 | 1.896372 |
| SEQ ID NO: 2 | 8.6287 | 2.8662 | −0.29969476 | 0.67431321 | | |
| SEQ ID NO: 3 | 6.0748 | 4.6331 | −0.15320348 | 0.34470782 | | |
| SEQ ID NO: 4 | 7.2020 | 2.5811 | −0.07827032 | 0.17610823 | | |
| SEQ ID NO: 5 | 9.2810 | 2.5943 | 0.07487556 | −0.16847 | | |
| SEQ ID NO: 6 | 9.1734 | 3.4356 | −0.06966885 | 0.15675492 | | |
| SEQ ID NO: 7 | 5.0310 | 2.5542 | −0.06962349 | 0.15665285 | | |
| SEQ ID NO: 8 | 5.1660 | 3.1202 | −0.05812405 | 0.13077912 | | |
| SEQ ID NO: 9 | 5.1174 | 2.7594 | −0.04657312 | 0.10478951 | | |
| SEQ ID NO: 10 | 6.3898 | 2.9469 | −0.04169181 | 0.09380658 | | |

when the expression level of the genes SEQ ID NO: 1-16 is measured

| 16 genes | Qci | Ji | V1i | V2i | T1 | T2 |
|---|---|---|---|---|---|---|
| SEQ ID NO: 1 | 8.1111 | 3.5040 | −0.398289229 | 0.896150764 | 0.540277 | 2.052201 |
| SEQ ID NO: 2 | 8.6287 | 2.8662 | −0.321772944 | 0.723989123 | | |
| SEQ ID NO: 3 | 6.0748 | 4.6331 | −0.175281658 | 0.394383731 | | |
| SEQ ID NO: 4 | 7.2020 | 2.5811 | −0.100348507 | 0.225784141 | | |
| SEQ ID NO: 5 | 9.2810 | 2.5943 | 0.096953738 | −0.218145911 | | |
| SEQ ID NO: 6 | 9.1734 | 3.4356 | −0.091747036 | 0.206430831 | | |
| SEQ ID NO: 7 | 5.0310 | 2.5542 | −0.091701673 | 0.206328765 | | |
| SEQ ID NO: 8 | 5.1660 | 3.1202 | −0.080202237 | 0.180455034 | | |
| SEQ ID NO: 9 | 5.1174 | 2.7594 | −0.068651299 | 0.154465422 | | |
| SEQ ID NO: 10 | 6.3898 | 2.9469 | −0.063769996 | 0.143482491 | | |
| SEQ ID NO: 11 | 8.8992 | 2.1597 | −0.020277623 | 0.045624651 | | |
| SEQ ID NO: 12 | 2.2380 | 2.3964 | −0.01079938 | 0.024298604 | | |
| SEQ ID NO: 13 | 6.9486 | 3.1865 | 0.008786792 | −0.019770281 | | |
| SEQ ID NO: 14 | 6.6286 | 2.6144 | −0.006607988 | 0.014867974 | | |
| SEQ ID NO: 15 | 13.6886 | 2.8714 | −0.006204653 | 0.013960469 | | |
| SEQ ID NO: 16 | 9.2036 | 3.3385 | −0.003597575 | 0.008094544 | | |

when the expression level of the genes SEQ ID NO: 1-22 is measured

| 22 genes | Qci | Ji | V1i | V2i | T1 | T2 |
|---|---|---|---|---|---|---|
| SEQ ID NO: 1 | 8.1111 | 3.5040 | −0.442610974 | 0.995874691 | 0.6255484 | 2.4838871 |
| SEQ ID NO: 2 | 8.6287 | 2.8662 | −0.366094689 | 0.82371305 | | |
| SEQ ID NO: 3 | 6.0748 | 4.6331 | −0.219603403 | 0.494107658 | | |
| SEQ ID NO: 4 | 7.2020 | 2.5811 | −0.144670252 | 0.325508068 | | |
| SEQ ID NO: 5 | 9.2810 | 2.5943 | 0.141275483 | −0.317869838 | | |
| SEQ ID NO: 6 | 9.1734 | 3.4356 | −0.136068781 | 0.306154758 | | |
| SEQ ID NO: 7 | 5.0310 | 2.5542 | −0.136023419 | 0.306052692 | | |
| SEQ ID NO: 8 | 5.1660 | 3.1202 | −0.124523982 | 0.28017896 | | |
| SEQ ID NO: 9 | 5.1174 | 2.7594 | −0.112973044 | 0.254189348 | | |
| SEQ ID NO: 10 | 6.3898 | 2.9469 | −0.108091741 | 0.243206417 | | |
| SEQ ID NO: 11 | 8.8992 | 2.1597 | −0.064599368 | 0.145348578 | | |
| SEQ ID NO: 12 | 2.2380 | 2.3964 | −0.055121125 | 0.124022531 | | |
| SEQ ID NO: 13 | 6.9486 | 3.1865 | 0.053108537 | −0.119494208 | | |
| SEQ ID NO: 14 | 6.6286 | 2.6144 | −0.050929734 | 0.114591901 | | |
| SEQ ID NO: 15 | 13.6886 | 2.8714 | −0.050526398 | 0.113684396 | | |
| SEQ ID NO: 16 | 9.2036 | 3.3385 | −0.04791932 | 0.107818471 | | |
| SEQ ID NO: 17 | 8.5740 | 3.4954 | 0.030451917 | −0.068516814 | | |
| SEQ ID NO: 18 | 10.7286 | 2.2250 | −0.029802867 | 0.067056452 | | |
| SEQ ID NO: 19 | 4.8529 | 3.1583 | −0.014836187 | 0.033381421 | | |
| SEQ ID NO: 20 | 8.0629 | 2.5087 | −0.010433641 | 0.023475692 | | |
| SEQ ID NO: 21 | 4.8347 | 4.4847 | −0.002903001 | 0.006531752 | | |
| SEQ ID NO: 22 | 6.3091 | 2.6921 | −0.002374696 | 0.005343066 | | |

The above matrices are appropriate to carry out the method according to the invention, when the prognosis of a patient, for which the expression level of said at least 3 genes according to the invention has been quantified by DNA CHIP, is evaluated.

The above values correspond to the values obtained for a determined cohort of reference patients having a WHO grade 2 or grade 3 glioma.

Applying the method disclosed in the Example, the skilled person could easily obtain similar results from any other determined cohort.

Certain preferred aspects and embodiments of the present invention will now be discussed in more detail:

### Direct Methods of Determining Quantitative Expression

More advantageously, the invention relates to the method previously defined, wherein the expression level of the genes is measured by a method allowing the determination of the amount of the mRNA or of the cDNA corresponding to said genes. Preferably said method is a quantitative method.

Levels of mRNA can be quantitatively measured by northern blotting which gives size and sequence information about the mRNA molecules. A sample of RNA is separated on an agarose gel and hybridized to a radio-labeled RNA probe that is complementary to the target sequence. The radio-labeled RNA is then detected by an autoradiograph. Northern blotting is widely used as the additional mRNA size information allows the discrimination of alternately spliced transcripts.

Another approach for measuring mRNA abundance is reverse transcription quantitative polymerase chain reaction (RT-PCR followed with qPCR). RT-PCR first generates a DNA template from the mRNA by reverse transcription, which is called cDNA. This cDNA template is then used for qPCR where the change in fluorescence of a probe changes as the DNA amplification process progresses. With a carefully constructed standard curve qPCR can produce an absolute measurement such as number of copies of mRNA, typically in units of copies per nanolitre of homogenized tissue or copies per cell. qPCR is very sensitive (detection of a single mRNA molecule is possible), but can be expensive due to the fluorescent probes required.

Northern blots and RT-qPCR are good for detecting whether a single gene or few genes are expressed.

Other methods known by one skilled in the art include DNA microarrays or technologies like Serial Analysis of Gene Expression (SAGE).

SAGE can provide a relative measure of the cellular concentration of different messenger RNAs. The great advantage of tag-based methods is the “open architecture”, allowing for the exact measurement of any transcript are present in cells, the sequence of said transcripts could be known or unknown.

In one another advantageous embodiment, the invention relates to the method defined above, wherein the expression level (e.g. quantitative expression value Qi) for a gene i is measured by any quantitative techniques like qRT-PCR or DNA Chip.

More preferably, the invention relates to the method defined above, wherein expression level (e.g. the quantitative expression value Qi) for a gene i is measured by a quantitative technique chosen among qRT-PCR and DNA Chip

The preferred quantitative techniques used to establish the expression level (e.g. quantitative value Qi) are qRT-PCR (hereafter qPCR) and DNA CHIP

qPCR is well known in the art, and can be carried out by using, in association with oligonucleotides allowing a specific amplification of the target gene, either with dyes or with reporter probe.

Both techniques are briefly summarized hereafter.

Real-Time PCR with Double-Stranded DNA-Binding Dyes as Reporters:

A DNA-binding dye binds to all double-stranded (ds)DNA in PCR, causing fluorescence of the dye. An increase in DNA product during PCR therefore leads to an increase in fluorescence intensity and is measured at each cycle, thus allowing DNA concentrations to be quantified.

However, dsDNA dyes such as SYBR Green will bind to all dsDNA PCR products, including nonspecific PCR products (such as Primer dimer). This can potentially interfere with or prevent accurate quantification of the intended target sequence.

The reaction is prepared as usual, with the addition of fluorescent dsDNA dye.

The reaction is run in a Real-time PCR instrument, and after each cycle, the levels of fluorescence are measured with a detector; the dye only fluoresces when bound to the dsDNA (i.e., the PCR product). With reference to a standard dilution, the dsDNA concentration in the PCR can be determined.

Like other real-time PCR methods, the values obtained do not have absolute units associated with them (i.e., mRNA copies/cell). As described above, a comparison of a measured DNA/RNA sample to a standard dilution will only give a fraction or ratio of the sample relative to the standard, allowing only relative comparisons between different tissues or experimental conditions. To ensure accuracy in the quantification, it is usually necessary to normalize expression of a target gene to a stably expressed gene (see below). This can correct possible differences in RNA quantity or quality across experimental samples.

Fluorescent Reporter Probe Method

Fluorescent reporter probes detect only the DNA containing the probe sequence; therefore, use of the reporter probe significantly increases specificity, and enables quantification even in the presence of non-specific DNA amplification. Fluorescent probes can be used in multiplex assays—for detection of several genes in the same reaction—based on specific probes with different-coloured labels, provided that all targeted genes are amplified with similar efficiency. The specificity of fluorescent reporter probes also prevents interference of measurements caused by primer dimers, which are undesirable potential by-products in PCR. However, fluorescent reporter probes do not prevent the inhibitory effect of the primer dimers, which may depress accumulation of the desired products in the reaction.

The method relies on a DNA-based probe with a fluorescent reporter at one end and a quencher of fluorescence at the opposite end of the probe. The close proximity of the reporter to the quencher prevents detection of its fluorescence; breakdown of the probe by the 5′ to 3′ exonuclease activity of the Taq polymerase breaks the reporter-quencher proximity and thus allows unquenched emission of fluorescence, which can be detected after excitation with a laser. An increase in the product targeted by the reporter probe at each PCR cycle therefore causes a proportional increase in fluorescence due to the breakdown of the probe and release of the reporter.

The PCR is prepared as usual, and the reporter probe is added.

During the annealing stage of the PCR both probe and primers anneal to the DNA target.

Polymerisation of a new DNA strand is initiated from the primers, and once the polymerase reaches the probe, its 5′-3′-exonuclease degrades the probe, physically separating the fluorescent reporter from the quencher, resulting in an increase in fluorescence.

Fluorescence is detected and measured in the real-time PCR thermocycler, and its geometric increase corresponding to exponential increase of the product is used to determine the threshold cycle (CT) in each reaction.

### Indirect Methods of Determining Quantitative Expression

In one embodiment the determining expression comprises contacting said sample with at least one antibody specific to a polypeptide (“target protein”) encoded by the relevant gene or a fragment thereof.

In one aspect of the present invention, the target protein can be detected using a binding moiety capable of specifically binding the marker protein. By way of example, the binding moiety may comprise a member of a ligand-receptor pair, i.e. a pair of molecules capable of having a specific binding interaction. The binding moiety may comprise, for example, a member of a specific binding pair, such as antibody-antigen, enzyme-substrate, nucleic acid-nucleic acid, protein-nucleic acid, protein-protein, or other specific binding pair known in the art. Binding proteins may be designed which have enhanced affinity for the target protein of the invention. Optionally, the binding moiety may be linked with a detectable label, such as an enzymatic, fluorescent, radioactive, phosphorescent, coloured particle label or spin label. The labelled complex may be detected, for example, visually or with the aid of a spectrophotometer or other detector.

A preferred embodiment of the present invention involves the use of a recognition agent, for example an antibody recognising the target protein of the invention, to con-tact a sample of glioma, and quantifying the response. Quantitative methods are well known to those skilled in the art and include radio-immunological methods or enzyme-linked antibody methods.

More specifically, examples of immunoassays are antibody capture assays, two-antibody sandwich assays, and antigen capture assays. In a sandwich immunoassay, two antibodies capable of binding the marker protein generally are used, e.g. one immobilised onto a solid support, and one free in solution and labelled with a detectable chemical compound. Examples of chemical labels that may be used for the second antibody include radioisotopes, fluorescent compounds, spin labels, coloured particles such as colloidal gold and coloured latex, and enzymes or other molecules that generate coloured or electrochemically active products when exposed to a reactant or enzyme substrate. When a sample containing the marker protein is placed in this system, the marker protein binds to both the immobilised antibody and the labelled antibody, to form a “sandwich” immune complex on the support's surface. The complexed protein is detected by washing away non-bound sample components and excess labelled antibody, and measuring the amount of labelled antibody complexed to protein on the support's surface. Alternatively, the antibody free in solution, which can be labelled with a chemical moiety, for example, a hapten, may be detected by a third antibody labelled with a detectable moiety which binds the free antibody or, for example, the hapten coupled thereto. Preferably, the immunoassay is a solid support-based immunoassay. Alternatively, the immunoassay may be one of the immunoprecipitation techniques known in the art, such as, for example, a nephelometric immunoassay or a turbidimetric immunoassay. When Western blot analysis or an immunoassay is used, preferably it includes a conjugated enzyme labelling technique.

Although the recognition agent will conveniently be an antibody, other recognition agents are known or may become available, and can be used in the present invention. For example, antigen binding domain fragments of antibodies, such as Fab fragments, can be used. Also, so-called RNA aptamers may be used. Therefore, unless the context specifically indicates otherwise, the term “antibody” as used herein is intended to include other recognition agents. Where antibodies are used, they may be polyclonal or monoclonal. Optionally, the antibody can be produced by a method such that it recognizes a preselected epitope from the target protein of the invention.

### Other Aspects and Embodiments

The invention also relates to a composition comprising oligonucleotides allowing the quantitative measure of the expression level of the genes of a set comprising at least 3 genes belonging to a group of 22 genes, said 22 genes comprising or being constituted by the respective nucleic acid sequences SEQ ID NO: 1 to 22,

wherein said at least 3 genes optionally comprise or are constituted by the respective nucleic acid sequences SEQ ID NO: 1 to 3,
said composition preferably consisting essentially of 1 to 20 oligonucleotides allowing the measure of the expression level of essentially at least the genes of a set comprising at least 3 genes belonging to a group of 22 genes,
for its use for determining, in vitro or ex vivo, from a biological sample of a subject afflicted by a WHO grade 2 or grade 3 glioma, the survival prognosis of said subject.

The composition according to the invention, as mentioned above, consists of pools, said pools consisting of 1, or 2 or 3, or 3 or 4 or 5 or 6 or 7 or 8 or 9 or 10 or 11 or 12 or 13 or 14 or 15 or 16 or 17 or 18 or 19 or 20 oligonucleotides that specifically hybridize with one gene of the group of 22 genes, said composition containing at least 3 pools.

As mentioned above, the composition consists of at least 3 pools, i.e. consists of 3, or 4, or 5, or 6, or 7, or 8, or 9, or 10, or 11, or, 12, or 13, or 14, or 15, or 16, or 17, or 18, or 19, or 20, or 21, or 22 pools, each pools consisting of 1, or 2 or 3, or 3 or 4 or 5 or 6 or 7 or 8 or 9 or 10 or 11 or 12 or 13 or 14 or 15 or 16 or 17 or 18 or 19 or 20 oligonucleotides that specifically hybridize with one gene of the group of 22 genes, the oligonucleotides comprised in each pool are not able to hybridize with the gene recognized by the oligonucleotides of another pool.

In other words, the composition according to the invention consists, in its minimal configuration, of at least 3 pools: a pool of oligonucleotides specifically hybridizing with the gene SEQ ID NO: 1, a pool of oligonucleotides specifically hybridizing with the gene SEQ ID NO: 2 and a pool of oligonucleotides specifically hybridizing with the gene SEQ ID NO: 3.

The oligonucleotides comprised in each pool, and that are specific of one of said at least 3 genes of the group of 22 genes, can be easily determined by the skilled person, since the nucleic acid sequence of each of the genes is known.

The structure of the nucleotide depends upon the technique which will be carried out to implement the method according to the invention.

For instance, if the method implements a qRT-PCR, each pool is preferably constituted by a couple of oligonucleotides consisting of 15-35 nucleotides, said oligonucleotides being reverse and anti-parallel, in order to carry out a PCR amplification. Advantageously, another oligonucleotide can be present, and will be used a probe (such as Taqman probe), said probe being used as quantifying indicator during the PCR amplification.

If the method is a DNA CHIP, each pool is preferably constituted by 5 to 15 oligonucleotides consisting of 15-60 nucleotides.

In one advantageous embodiment, the oligonucleotide probes used in the invention are the following ones:

| gene | Probe set number | Probe sequence | SEQ ID |
|---|---|---|---|
| CHI3L1 | HG-U133_PLUS_2: | TCACCAATGCCATCAAGGATGCACT | SEQ ID NO 89 |
| | 209396_S_AT | CAAGGATGCACTCGCTGCAACGTAG | SEQ ID NO 90 |
| | | CACACAGCACGGGGGCCAAGGATGC | SEQ ID NO 91 |
| | | TGCAGAGGTCCACAACACACAGATT | SEQ ID NO 92 |
| | | CACAGATTTGAGCTCAGCCCTGGTG | SEQ ID NO 93 |
| | | CCCTAGCCCTCCTTATCAAAGGACA | SEQ ID NO 94 |
| | | AAGGACACCATTTTGGCAAGCTCTA | SEQ ID NO 95 |
| | | GGCAAGCTCTATCACCAAGGAGCCA | SEQ ID NO 96 |
| | | ATCCTACAAGACACAGTGACCATAC | SEQ ID NO 97 |
| | | AGTGACCATACTAATTATACCCCCT | SEQ ID NO 98 |
| | | GCAAAGCCAGCTTGAAACCTTCACT | SEQ ID NO 99 |
| IGFBP2 | HG-U133_PLUS_2: | ATCCCCAACTGTGACAAGCATGGCC | SEQ ID NO 100 |
| | 202718_AT | TGACAAGCATGGCCTGTACAACCTC | SEQ ID NO 101 |
| | | GTACAACCTCAAACAGTGCAAGATG | SEQ ID NO 102 |
| | | GCAAGATGTCTCTGAACGGGCAGCG | SEQ ID NO 103 |
| | | ACGGGCAGCGTGGGGAGTGCTGGTG | SEQ ID NO 104 |
| | | GAACCCCAACACCGGGAAGCTGATC | SEQ ID NO 105 |
| | | CACCGGGAAGCTGATCCAGGGAGCC | SEQ ID NO 106 |
| | | CATCCGGGGGGACCCCGAGTGTCAT | SEQ ID NO 107 |
| | | GAGTGTCATCTCTTCTACAATGAGC | SEQ ID NO 108 |
| | | GCACACCCAGCGGATGCAGTAGACC | SEQ ID NO 109 |
| | | GAAAACGGAGAGTGCTTGGGTGGTG | SEQ ID NO 110 |
| POSTN | HG-U133_PLUS_2: | AAATTGTGGAGTTAGCCTCCTGTGG | SEQ ID NO 111 |
| | 210809_S_AT | GTGGAGTTAGCCTCCTGTGGTAAAG | SEQ ID NO 112 |
| | | TTACACCCTTTTTCATCTTGACATT | SEQ ID NO 113 |
| | | GTTCTGGCTAACTTTGGAATCCATT | SEQ ID NO 114 |
| | | AGAGTTGTGAACTGTTATCCCATTG | SEQ ID NO 115 |
| | | TTATCCCATTGAAAAGACCGAGCCT | SEQ ID NO 116 |
| | | GACCGAGCCTTGTATGTATGTTATG | SEQ ID NO 117 |
| | | AAATGCACGCAAGCCATTATCTCTC | SEQ ID NO 118 |
| | | AGCCATTATCTCTCCATGGGAAGCT | SEQ ID NO 119 |
| | | AGGCTTTGCACATTTCTATATGAGT | SEQ ID NO 120 |
| | | GTTTGTCATATGCTTCTTGCAATGC | SEQ ID NO 121 |
| HSPG2 | HG-U133_PLUS_2: | TCCCTCCCTCAGGGGCTGTAAGGGA | SEQ ID NO 122 |
| | 201655_S_AT | TCAGGGGCTGTAAGGGAAGGCCCAC | SEQ ID NO 123 |
| | | ACTCCTCCAACAGACAACGGACGGA | SEQ ID NO 124 |
| | | GACAACGGACGGACGGATGCCGCTG | SEQ ID NO 125 |
| | | ATGCCGCTGGTGCTCAGGAAGAGCT | SEQ ID NO 126 |
| | | GCTCAGGAAGAGCTAGTGCCTTAGG | SEQ ID NO 127 |
| | | GGAAGAGCTAGTGCCTTAGGTGGGG | SEQ ID NO 128 |
| | | AGAGCTAGTGCCTTAGGTGGGGGAA | SEQ ID NO 129 |
| | | GGAAGGCAGGACTCACGACTGAGAG | SEQ ID NO 130 |
| | | GGCAGGACTCACGACTGAGAGAGAG | SEQ ID NO 131 |
| | | GCCCCCAGACTGTGGGGTTGGGACG | SEQ ID NO 132 |
| BMP2 | HG-U133_PLUS_2: | TATCGGGTTTGTACATAATTTTCCA | SEQ ID NO 133 |
| | 205289_AT | AATTGTAGTTGTTTTCAGTTGTGTG | SEQ ID NO 134 |
| | | GGAAGGTTACTCTGGCAAAGTGCTT | SEQ ID NO 135 |
| | | GTTTGCTTTTTTGCAGTGCTACTGT | SEQ ID NO 136 |
| | | GTGCTACTGTTGAGTTCACAAGTTC | SEQ ID NO 137 |
| | | GTGGATAATCCACTCTGCTGACTTT | SEQ ID NO 138 |
| | | AGAACCAGACATTGCTGATCTATTA | SEQ ID NO 139 |
| | | CTATTATAGAAACTCTCCTCCTGCC | SEQ ID NO 140 |
| | | TCCTCCTGCCCCTTAATTTACAGAA | SEQ ID NO 141 |
| | | TTTCCTAAATTAGTGATCCCTTCAA | SEQ ID NO 142 |
| | | GGGGCTGATCTGGCCAAAGTATTCA | SEQ ID NO 143 |
| COL1A1 | HG-U133_PLUS_2: | TGGGAGACAATTTCACATGGACTTT | SEQ ID NO 144 |
| | 1556499_s_at | GAGACAATTTCACATGGACTTTGGA | SEQ ID NO 145 |
| | | ACAATTTCACATGGACTTTGGAAAA | SEQ ID NO 146 |
| | | TTCCTTTGCATTCATCTCTCAAACT | SEQ ID NO 147 |
| | | TCCTTTGCATTCATCTCTCAAACTT | SEQ ID NO 148 |
| | | TTTGCATTCATCTCTCAAACTTAGT | SEQ ID NO 149 |
| | | TGCATTCATCTCTCAAACTTAGTTT | SEQ ID NO 150 |
| | | CATTCATCTCTCAAACTTAGTTTTT | SEQ ID NO 151 |
| | | ATCTCTCAAACTTAGTTTTTATCTT | SEQ ID NO 152 |
| | | TTTTTATCTTTGACCAACCGAACAT | SEQ ID NO 153 |
| | | TTTATCTTTGACCAACCGAACATGA | SEQ ID NO 154 |
| NEK2 | HG-U133_PLUS_2: | GCTGTAGTGTTGAATACTTGGCCCC | SEQ ID NO 155 |
| | 204641_AT | TGAATACTTGGCCCCATGAGCCATG | SEQ ID NO 156 |
| | | GCCATGCCTTTCTGTATAGTACACA | SEQ ID NO 157 |
| | | GATATTTCGGAATTGGTTTTACTGT | SEQ ID NO 158 |
| | | TTGGTTGGGCTTTTAATCCTGTGTG | SEQ ID NO 159 |
| | | GTAGCACTCACTGAATAGTTTTAAA | SEQ ID NO 160 |
| | | GGTATGCTTACAATTGTCATGTCTA | SEQ ID NO 161 |
| | | ATTAATACCATGACATCTTGCTTAT | SEQ ID NO 162 |
| | | AAATATTCCATTGCTCTGTAGTTCA | SEQ ID NO 163 |
| | | CTCTGTAGTTCAAATCTGTTAGCTT | SEQ ID NO 164 |
| | | TGAGCTGTCTGTCATTTACCTACTT | SEQ ID NO 165 |
| DLG7 | HG-U133_PLUS_2: | GTGAGAGAATGAGTTTGCCTCTTCT | SEQ ID NO 166 |
| | 203764_AT | GGATGTTTTGATGAGTAGCCCTGAA | SEQ ID NO 167 |
| | | AAAGTCTCACTACTGAATGCCACCT | SEQ ID NO 168 |
| | | CCACCTTCTTGATTCACCAGGTCTA | SEQ ID NO 169 |
| | | GCAGTAATCCATTTACTCAGCTGGA | SEQ ID NO 170 |
| | | GAGACATCAAGAACATGCCAGACAC | SEQ ID NO 171 |
| | | ATGCCAGACACATTTCTTTTGGTGG | SEQ ID NO 172 |
| | | TGGTAACCTGATTACTTTTTCACCT | SEQ ID NO 173 |
| | | ACTTTTTCACCTCTACAACCAGGAG | SEQ ID NO 174 |
| | | ATTTGTGTTCACTTCTATAGCATAT | SEQ ID NO 175 |
| | | GATATACTCTTTCTCAAGGGAAGTG | SEQ ID NO 176 |
| FOXM1 | HG-U133_PLUS_2: | AGCTGACTTGGAAACACGGGGAGGT | SEQ ID NO 177 |
| | 214148_AT | CAAGCAGATCCACTTGTCTGGGTCC | SEQ ID NO 178 |
| | | GTCTGGGTCCCTGCAGTGAAGAACC | SEQ ID NO 179 |
| | | AGAACCCAAGATCCAGGTACCTCAG | SEQ ID NO 180 |
| | | AGAAACCGTGCACTGCAGGTCTTCC | SEQ ID NO 181 |
| | | ATTTCTTCCTCCTTGATAGTCTGAA | SEQ ID NO 182 |
| | | AGAAAGAGGAGCTATCCCCTCCTCA | SEQ ID NO 183 |
| | | CTCCTCAGCTAGCAGCACCTGAAAG | SEQ ID NO 184 |
| | | GAACCAACGGTCACCAGACAGGACG | SEQ ID NO 185 |
| | | ACATACGGGTTCTGATCCTCTTTGT | SEQ ID NO 186 |
| | | GATCCTCTTTGTGTCGTTTTGAAGT | SEQ ID NO 187 |
| BIRC5 | HG-U133_PLUS_2: | GCTCCTCTACTGTTTAACAACATGG | SEQ ID NO 188 |
| | 202095_S_AT | AAGCACAAAGCCATTCTAAGTCATT | SEQ ID NO 189 |
| | | GGAAGCGTCTGGCAGATACTCCTTT | SEQ ID NO 190 |
| | | TGGCAGATACTCCTTTTGCCACTGC | SEQ ID NO 191 |
| | | TGATTAGACAGGCCCAGTGAGCCGC | SEQ ID NO 192 |
| | | AATGACTTGGCTCGATGCTGTGGGG | SEQ ID NO 193 |
| | | TCACGTTCTCCACACGGGGGAGAGA | SEQ ID NO 194 |
| | | TCCCGCAGGGCTGAAGTCTGGCGTA | SEQ ID NO 195 |
| | | GATGATGGATTTGATTCGCCCTCCT | SEQ ID NO 196 |
| | | TACAGCTTCGCTGGAAACCTCTGGA | SEQ ID NO 197 |
| | | GGAAACCTCTGGAGGTCATCTCGGC | SEQ ID NO 198 |
| PLK1 | HG-U133_PLUS_2: | TGGGTTATGCCCAACATCTGCTTTC | SEQ ID NO 199 |
| | 1555900_AT | TGAGCAGCTCCCAATGAGAACCCTG | SEQ ID NO 200 |
| | | GAGAACCCTGAACACTGAGTCTGTA | SEQ ID NO 201 |
| | | AGTCTGTAATGAGCTTCCCTTGTAT | SEQ ID NO 202 |
| | | GAGCTTCCCTTGTATACAACATTGC | SEQ ID NO 203 |
| | | CAACATTGCACATGGGTTGTCACAA | SEQ ID NO 204 |
| | | GTCACAACTGATTGCTGGAGGAATT | SEQ ID NO 205 |
| | | AATTGTGTCCTATGTGACTCTGCTG | SEQ ID NO 206 |
| | | ACTGTGGGAGGCTTACACCTGGTTT | SEQ ID NO 207 |
| | | TGGACTTTGTCCATGCGCTTTTTTC | SEQ ID NO 208 |
| | | TTGCTGATTTTGCTTCCTAGCCTTT | SEQ ID NO 209 |
| NKX6-1 | HG-U133_PLUS_2: | TCTGGCCCGGAGTGATGCAGAGCCC | SEQ ID NO 210 |
| | 221366_AT | GTACCCCTCATCAAGGATCCATTTT | SEQ ID NO 211 |
| | | AGAGAAAACACACGAGACCCACTTT | SEQ ID NO 212 |
| | | TTTTTCCGGACAGCAGATCTTCGCC | SEQ ID NO 213 |
| | | TACTTGGCGGGGCCCGAGAGGGCTC | SEQ ID NO 214 |
| | | CTCGTTTGGCCTATTCGTTGGGGAT | SEQ ID NO 215 |
| | | GAGTCAGGTCAAGGTCTGGTTCCAG | SEQ ID NO 216 |
| | | GAAGCAGGACTCGGAGACAGAGCGC | SEQ ID NO 217 |
| | | GACTACAATAAGCCTCTGGATCCCA | SEQ ID NO 218 |
| | | GAAGAAGCACAAGTCCAGCAGCGGC | SEQ ID NO 219 |
| | | TCCGAGCCGGAGAGCTCATCCTGAA | SEQ ID NO 220 |
| NRG3 | HG-U133_PLUS_2: | CATGTGTTCATTGTGCGTATGTGTG | SEQ ID NO 221 |
| | 229233_AT | GTGCATGTGTGCGCGTATTACGCTT | SEQ ID NO 222 |
| | | TTACGCTTGCTAAAATTTGTTCTGA | SEQ ID NO 223 |
| | | AGGTCACTTGCATGGTGGGGTCGTA | SEQ ID NO 224 |
| | | GGTCGTATAAAACCCTTGACACTGT | SEQ ID NO 225 |
| | | GACACTGTCTAGACCATTTTCTGAT | SEQ ID NO 226 |
| | | GAGAGGATCAACTATTGGCTCATTA | SEQ ID NO 227 |
| | | TAGCAAGTCTGCTATGTGTGGACCA | SEQ ID NO 228 |
| | | GCTTCGGCTTCTGTGGTTAGTATGG | SEQ ID NO 229 |
| | | AATACCCAGACTATTCAGTTCACAA | SEQ ID NO 230 |
| | | CTATTCAGTTCACAAGAAGCCCCCC | SEQ ID NO 231 |
| BUB1B | HG-U133_PLUS_2: | TTCTTTGTGCGGATTCTGAATGCCA | SEQ ID NO 232 |
| | 203755_AT | TGGGGTTTTTGACACTACATTCCAA | SEQ ID NO 233 |
| | | GTTAACTAGTCCTGGGGCTTTGCTC | SEQ ID NO 234 |
| | | GGGGCTTTGCTCTTTCAGTGAGCTA | SEQ ID NO 235 |
| | | GAGCTAGGCAATCAAGTCTCACAGA | SEQ ID NO 236 |
| | | GTCTCACAGATTGCTGCCTCAGAGC | SEQ ID NO 237 |
| | | GGACACATTTAGATGCACTACCATT | SEQ ID NO 238 |
| | | CACTACCATTGCTGTTCTACTTTTT | SEQ ID NO 239 |
| | | GGTACAGGTATATTTTGACGTCACT | SEQ ID NO 240 |
| | | GGCCTTGTCTAACTTTTGTGAAGAA | SEQ ID NO 241 |
| | | GTTCTCTTATGATCACCATGTATTT | SEQ ID NO 242 |
| VIM | HG-U133_PLUS_2: | TGTGGATGTTTCCAAGCCTGACCTC | SEQ ID NO 243 |
| | 201426_S_AT | TGCCCTGCGTGACGTACGTCAGCAA | SEQ ID NO 244 |
| | | GTGTGGCTGCCAAGAACCTGCAGGA | SEQ ID NO 245 |
| | | AGTACCGGAGACAGGTGCAGTCCCT | SEQ ID NO 246 |
| | | GCAGTCCCTCACCTGTGAAGTGGAT | SEQ ID NO 247 |
| | | TGAGTCCCTGGAACGCCAGATGCGT | SEQ ID NO 248 |
| | | GAGAACTTTGCCGTTGAAGCTGCTA | SEQ ID NO 249 |
| | | GAAGCTGCTAACTACCAAGACACTA | SEQ ID NO 250 |
| | | CACTATTGGCCGCCTGCAGGATGAG | SEQ ID NO 251 |
| | | GTCACCTTCGTGAATACCAAGACCT | SEQ ID NO 252 |
| | | GCCCTTGACATTGAGATTGCCACCT | SEQ ID NO 253 |
| TNC | HG-U133_PLUS_2: | TTTTACCAAAGCATCAATACAACCA | SEQ ID NO 254 |
| | 201645_AT | CGGTCCACACCTGGGCATTTGGTGA | SEQ ID NO 255 |
| | | TCAAAGCTGACCATGGATCCCTGGG | SEQ ID NO 256 |
| | | TTGCACCAAAGACATCAGTCTCCAA | SEQ ID NO 257 |
| | | CATCAGTCTCCAACATGTTTCTGTT | SEQ ID NO 258 |
| | | ATCGCAATAGTTTTTTACTTCTCTT | SEQ ID NO 259 |
| | | TTACTTCTCTTAGGTGGCTCTGGGA | SEQ ID NO 260 |
| | | GAACCAGCCGTATTTTACATGAAGC | SEQ ID NO 261 |
| | | ATGTGTCATTGGAAGCCATCCCTTT | SEQ ID NO 262 |
| | | TCAAGAGATCTTTCTTTCCAAAACA | SEQ ID NO 263 |
| | | ACATTTCTGGACAGTACCTGATTGT | SEQ ID NO 264 |
| DLL3 | HG-U133_PLUS_2: | TCCCGGCTACATGGGAGCGCGGTGT | SEQ ID NO 265 |
| | 219537_X_AT | TGGCCACTCCCAGGATGCTGGGTCT | SEQ ID NO 266 |
| | | GATGCACTCAACAACCTAAGGACGC | SEQ ID NO 267 |
| | | GACGCAGGAGGGTTCCGGGGATGGT | SEQ ID NO 268 |
| | | GTCCGAGCTCGTCCGTAGATTGGAA | SEQ ID NO 269 |
| | | AATCGCCCTGAAGATGTAGACCCTC | SEQ ID NO 270 |
| | | GGATTTATGTCATATCTGCTCCTTC | SEQ ID NO 271 |
| | | CTTCCATCTACGCTCGGGAGGTAGC | SEQ ID NO 272 |
| | | CTTCCTCGATTCTGTCCGTGAAATG | SEQ ID NO 273 |
| | | TTTAAGCCCATTTTCAGTTCTAACT | SEQ ID NO 274 |
| | | TTACTTTCATCCTATTTTGCATCCC | SEQ ID NO 275 |
| JAG1 | HG-U133_PLUS_2: | TTTGTTTTTCTGCTTTAGACTTGAA | SEQ ID NO 276 |
| | 209099_X_AT | GAGACAGGCAGGTGATCTGCTGCAG | SEQ ID NO 277 |
| | | GGAAGCACACCAATCTGACTTTGTA | SEQ ID NO 278 |
| | | GATTTCTTTTCACCATTCGTACATA | SEQ ID NO 279 |
| | | GAACCACTTGTAGATTTGATTTTTT | SEQ ID NO 280 |
| | | AGATCACTGTTTAGATTTGCCATAG | SEQ ID NO 281 |
| | | TTTGCCATAGAGTACACTGCCTGCC | SEQ ID NO 282 |
| | | GTACACTGCCTGCCTTAAGTGAGGA | SEQ ID NO 283 |
| | | AGAGTAATCTTGTTGGTTCACCATT | SEQ ID NO 284 |
| | | GATACTTTGTATTGTCCTATTAGTG | SEQ ID NO 285 |
| | | GCATCTTTGATGTGTTGTTCTTGGC | SEQ ID NO 286 |
| KI67 | HG-U133_PLUS_2: | AAACTGGCTCCTAATCTCCAGCTTT | SEQ ID NO 287 |
| | 212020_S_AT | AGCTTCGGAAGTTTACTGGCTCTGC | SEQ ID NO 288 |
| | | TTCTTTCTGACTCTATCTGGCAGCC | SEQ ID NO 289 |
| | | GTACTCTGTAAAGCATCATCATCCT | SEQ ID NO 290 |
| | | GAGAGACTGAGCACTCAGCACCTTC | SEQ ID NO 291 |
| | | TTTCAGGATCGCTTCCTTGTGAGCC | SEQ ID NO 292 |
| | | TCTTTCTCCAGCTTCAGACTTGTAG | SEQ ID NO 293 |
| | | AACTCGTTCATCTTCATTTACTTTC | SEQ ID NO 294 |
| | | CAAATCAGAGAATAGCCCGCCATCC | SEQ ID NO 295 |
| | | CACCCACCTTGCCAGGTGCAGGTGA | SEQ ID NO 296 |
| | | GTTTCCCCAGTGTCTGGCGGGGAGC | SEQ ID NO 297 |
| EZH2 | HG-U133_PLUS_2: | AAATTCGTTTTGCAAATCATTCGGT | SEQ ID NO 298 |
| | 203358_S_AT | AAATCATTCGGTAAATCCAAACTGC | SEQ ID NO 299 |
| | | GATCACAGGATAGGTATTTTTGCCA | SEQ ID NO 300 |
| | | TTTTGCCAAGAGAGCCATCCAGACT | SEQ ID NO 301 |
| | | CCATCCAGACTGGCGAAGAGCTGTT | SEQ ID NO 302 |
| | | GAAACAGCTGCCTTAGCTTCAGGAA | SEQ ID NO 303 |
| | | CTGCCTTAGCTTCAGGAACCTCGAG | SEQ ID NO 304 |
| | | TCAGGAACCTCGAGTACTGTGGGCA | SEQ ID NO 305 |
| | | GCCTTCTCACCAGCTGCAAAGTGTT | SEQ ID NO 306 |
| | | CAAAGTGTTTTGTACCAGTGAATTT | SEQ ID NO 307 |
| | | GCAGTATGGTACATTTTTCAACTTT | SEQ ID NO 308 |
| BUB1 | HG-U133_PLUS_2: | GAAGATGATTTATCTGCTGGCTTGG | SEQ ID NO 309 |
| | 209642_AT | TGCTGGCTTGGCACTGATTGACCTG | SEQ ID NO 310 |
| | | GATGCTCAGCAACAAACCATGGAAC | SEQ ID NO 311 |
| | | GAACTACCAGATCGATTACTTTGGG | SEQ ID NO 312 |
| | | ATTACTTTGGGGTTGCTGCAACAGT | SEQ ID NO 313 |
| | | CATGCTCTTTGGCACTTACATGAAA | SEQ ID NO 314 |
| | | GAGAGTGTAAGCCTGAAGGTCTTTT | SEQ ID NO 315 |
| | | TTAGAAGGCTTCCTCATTTGGATAT | SEQ ID NO 316 |
| | | AATATTCCAGATTGTCATCATCTTC | SEQ ID NO 317 |
| | | GATTAGGGCCCTACGTAATAGGCTA | SEQ ID NO 318 |
| | | TAATAGGCTAATTGTACTGCTCTTA | SEQ ID NO 319 |
| AURKA | HG-U133_PLUS_2: | CCCTCAATCTAGAACGCTACACAAG | SEQ ID NO 320 |
| | 208079_S_AT | AAATAGGAACACGTGCTCTACCTCC | SEQ ID NO 321 |
| | | GTGCTCTACCTCCATTTAGGGATTT | SEQ ID NO 322 |
| | | CTACCTCCATTTAGGGATTTGCTTG | SEQ ID NO 323 |
| | | TTAGGGATTTGCTTGGGATACAGAA | SEQ ID NO 324 |
| | | GGGATACAGAAGAGGCCATGTGTCT | SEQ ID NO 325 |
| | | GAAGAGGCCATGTGTCTCAGAGCTG | SEQ ID NO 326 |
| | | GAGGCCATGTGTCTCAGAGCTGTTA | SEQ ID NO 327 |
| | | GTGTCTCAGAGCTGTTAAGGGCTTA | SEQ ID NO 328 |
| | | CAGAGCTGTTAAGGGCTTATTTTTT | SEQ ID NO 329 |
| | | CATTGGAGTCATAGCATGTGTGTAA | SEQ ID NO 330 |

Table 3 represents the probes sequences, their respective SEQ ID and the Affymetrix probe sets comprising them. The target gene is also indicated.

In one advantageous embodiment, the invention relates to a composition as defined above, wherein said set comprise at least 7 genes belonging to said group of 22 genes, said at least 7 genes comprising or being constituted by the respective nucleic acid sequences SEQ ID NO: 1 to 7.

In this configuration, the composition according to the invention consists of at least 7 pools: a pool of oligonucleotides specifically hybridizing with the gene SEQ ID NO: 1, a pool of oligonucleotides specifically hybridizing with the gene SEQ ID NO: 2, a pool of oligonucleotides specifically hybridizing with the gene SEQ ID NO: 3, a pool of oligonucleotides specifically hybridizing with the gene SEQ ID NO: 4, a pool of oligonucleotides specifically hybridizing with the gene SEQ ID NO: 5, a pool of oligonucleotides specifically hybridizing with the gene SEQ ID NO: 6, and a pool of oligonucleotides specifically hybridizing with the gene SEQ ID NO: 7.

In one advantageous embodiment, the invention relates to a composition as defined above, wherein said set comprise at least 9 genes belonging to a said group of 22 genes, said at least 9 genes comprising or being constituted by the respective nucleic acid sequences SEQ ID NO: 1 to 9.

In this configuration, the composition according to the invention consists of at least 9 pools: a pool of oligonucleotides specifically hybridizing with the gene SEQ ID NO: 1, a pool of oligonucleotides specifically hybridizing with the gene SEQ ID NO: 2, a pool of oligonucleotides specifically hybridizing with the gene SEQ ID NO: 3, a pool of oligonucleotides specifically hybridizing with the gene SEQ ID NO: 4, a pool of oligonucleotides specifically hybridizing with the gene SEQ ID NO: 5, a pool of oligonucleotides specifically hybridizing with the gene SEQ ID NO: 6, a pool of oligonucleotides specifically hybridizing with the gene SEQ ID NO: 7, a pool of oligonucleotides specifically hybridizing with the gene SEQ ID NO: 8 and a pool of oligonucleotides specifically hybridizing with the gene SEQ ID NO: 9.

The invention relates to a composition as defined above, wherein said set comprise at least 10 genes belonging to said group of 22 genes, said at least 10 genes comprising or being constituted by the respective nucleic acid sequences SEQ ID NO: 1 to 10.

In this configuration, the composition according to the invention consists of at least 10 pools: a pool of oligonucleotides specifically hybridizing with the gene SEQ ID NO: 1, a pool of oligonucleotides specifically hybridizing with the gene SEQ ID NO: 2, a pool of oligonucleotides specifically hybridizing with the gene SEQ ID NO: 3, a pool of oligonucleotides specifically hybridizing with the gene SEQ ID NO: 4, a pool of oligonucleotides specifically hybridizing with the gene SEQ ID NO: 5, a pool of oligonucleotides specifically hybridizing with the gene SEQ ID NO: 6, a pool of oligonucleotides specifically hybridizing with the gene SEQ ID NO: 7, a pool of oligonucleotides specifically hybridizing with the gene SEQ ID NO: 8, a pool of oligonucleotides specifically hybridizing with the gene SEQ ID NO: 9 and a pool of oligonucleotides specifically hybridizing with the gene SEQ ID NO: 10.

The invention relates to a composition as defined above, wherein said set comprise at least 16 genes belonging to said group of 22 genes, said at least 16 genes comprising or being constituted by the respective nucleic acid sequences SEQ ID NO: 1 to 16.

In this configuration, the composition according to the invention consists of at least 16 pools: a pool of oligonucleotides specifically hybridizing with the gene SEQ ID NO: 1, a pool of oligonucleotides specifically hybridizing with the gene SEQ ID NO: 2, a pool of oligonucleotides specifically hybridizing with the gene SEQ ID NO: 3, a pool of oligonucleotides specifically hybridizing with the gene SEQ ID NO: 4, a pool of oligonucleotides specifically hybridizing with the gene SEQ ID NO: 5, a pool of oligonucleotides specifically hybridizing with the gene SEQ ID NO: 6, a pool of oligonucleotides specifically hybridizing with the gene SEQ ID NO: 7, a pool of oligonucleotides specifically hybridizing with the gene SEQ ID NO: 8, a pool of oligonucleotides specifically hybridizing with the gene SEQ ID NO: 9, a pool of oligonucleotides specifically hybridizing with the gene SEQ ID NO: 10, a pool of oligonucleotides specifically hybridizing with the gene SEQ ID NO: 11, a pool of oligonucleotides specifically hybridizing with the gene SEQ ID NO: 12, a pool of oligonucleotides specifically hybridizing with the gene SEQ ID NO: 13, a pool of oligonucleotides specifically hybridizing with the gene SEQ ID NO: 14, a pool of oligonucleotides specifically hybridizing with the gene SEQ ID NO: 15 and a pool of oligonucleotides specifically hybridizing with the gene SEQ ID NO: 16.

In one advantageous embodiment, the invention relates to a composition as defined above, wherein said set consists of all the genes of said group of 22 genes.

In this configuration, the composition according to the invention consists of 22 pools: a pool of oligonucleotides specifically hybridizing with the gene SEQ ID NO: 1, a pool of oligonucleotides specifically hybridizing with the gene SEQ ID NO: 2, a pool of oligonucleotides specifically hybridizing with the gene SEQ ID NO: 3, a pool of oligonucleotides specifically hybridizing with the gene SEQ ID NO: 4, a pool of oligonucleotides specifically hybridizing with the gene SEQ ID NO: 5, a pool of oligonucleotides specifically hybridizing with the gene SEQ ID NO: 6, a pool of oligonucleotides specifically hybridizing with the gene SEQ ID NO: 7, a pool of oligonucleotides specifically hybridizing with the gene SEQ ID NO: 8, a pool of oligonucleotides specifically hybridizing with the gene SEQ ID NO: 9, a pool of oligonucleotides specifically hybridizing with the gene SEQ ID NO: 10, a pool of oligonucleotides specifically hybridizing with the gene SEQ ID NO: 11, a pool of oligonucleotides specifically hybridizing with the gene SEQ ID NO: 12, a pool of oligonucleotides specifically hybridizing with the gene SEQ ID NO: 13, a pool of oligonucleotides specifically hybridizing with the gene SEQ ID NO: 14, a pool of oligonucleotides specifically hybridizing with the gene SEQ ID NO: 15, a pool of oligonucleotides specifically hybridizing with the gene SEQ ID NO: 16, a pool of oligonucleotides specifically hybridizing with the gene SEQ ID NO: 17, a pool of oligonucleotides specifically hybridizing with the gene SEQ ID NO: 18, a pool of oligonucleotides specifically hybridizing with the gene SEQ ID NO: 19, a pool of oligonucleotides specifically hybridizing with the gene SEQ ID NO: 20, a pool of oligonucleotides specifically hybridizing with the gene SEQ ID NO: 21 and a pool of oligonucleotides specifically hybridizing with the gene SEQ ID NO: 22.

In one advantageous embodiment, the composition according to the invention as defined above may further comprise one or more pools containing oligonucleotides allowing the detection of control genes, such as Actin, TBP, tubuline and so on. The above list is not limitative.

The skill person could easily determine what type of control gene may be used.

In still another advantageous embodiment, the invention relates to a composition according to the previous definition, wherein said composition comprises at least a pair of oligonucleotides allowing the measure of the expression of the genes of said set of genes belonging to said group of 22 genes.

In this advantageous embodiment, each pool as defined above comprise a pair of oligonucleotides, said pair of oligonucleotides being such that they allow the PCR amplification of a determined gene.

This advantageous embodiment of the composition of the invention is particularly advantageous when PCR is used to quantify the expression level of the at least 3 genes according to the invention. However, this could be also used to carry out the method according to the invention by measure the expression level of the at least 3 genes by DNA-CHIP.

In a more advantageous embodiment, the invention relates to the composition defined above, wherein said composition comprises at least the oligonucleotides SEQ ID NO: 23-28, preferably at least the oligonucleotides SEQ ID NO: 23-40, more preferably at least the oligonucleotides SEQ ID NO: 23-42, more preferably at least the oligonucleotides SEQ ID NO: 23-54, chosen among the group consisting of the oligonucleotides SEQ ID NO: 23-66, and in particular said composition comprises the oligonucleotides SEQ ID NO: 23-66,

said oligonucleotides being such that:

SEQ ID NO: 23 and SEQ ID NO: 24 specifically hybridize with the gene SEQ ID NO: 1,

SEQ ID NO: 25 and SEQ ID NO: 26 specifically hybridize with the gene SEQ ID NO: 2,

SEQ ID NO: 27 and SEQ ID NO: 28 specifically hybridize with the gene SEQ ID NO: 3,

SEQ ID NO: 29 and SEQ ID NO: 30 specifically hybridize with the gene SEQ ID NO: 4,

SEQ ID NO: 31 and SEQ ID NO: 32 specifically hybridize with the gene SEQ ID NO: 5,

SEQ ID NO: 33 and SEQ ID NO: 34 specifically hybridize with the gene SEQ ID NO: 6,

SEQ ID NO: 35 and SEQ ID NO: 36 specifically hybridize with the gene SEQ ID NO: 7,

SEQ ID NO: 37 and SEQ ID NO: 38 specifically hybridize with the gene SEQ ID NO: 8,

SEQ ID NO: 39 and SEQ ID NO: 40 specifically hybridize with the gene SEQ ID NO: 9,

SEQ ID NO: 41 and SEQ ID NO: 42 specifically hybridize with the gene SEQ ID NO: 10,

SEQ ID NO: 43 and SEQ ID NO: 44 specifically hybridize with the gene SEQ ID NO: 11,

SEQ ID NO: 45 and SEQ ID NO: 46 specifically hybridize with the gene SEQ ID NO: 12,

SEQ ID NO: 47 and SEQ ID NO: 48 specifically hybridize with the gene SEQ ID NO: 13,

SEQ ID NO: 49 and SEQ ID NO: 50 specifically hybridize with the gene SEQ ID NO: 14,

SEQ ID NO: 51 and SEQ ID NO: 52 specifically hybridize with the gene SEQ ID NO: 15,

SEQ ID NO: 53 and SEQ ID NO: 54 specifically hybridize with the gene SEQ ID NO: 16,

SEQ ID NO: 55 and SEQ ID NO: 56 specifically hybridize with the gene SEQ ID NO: 17,

SEQ ID NO: 57 and SEQ ID NO: 58 specifically hybridize with the gene SEQ ID NO: 18,

SEQ ID NO: 59 and SEQ ID NO: 60 specifically hybridize with the gene SEQ ID NO: 19,

SEQ ID NO: 61 and SEQ ID NO: 62 specifically hybridize with the gene SEQ ID NO: 20,

SEQ ID NO: 63 and SEQ ID NO: 64 specifically hybridize with the gene SEQ ID NO: 21, and

SEQ ID NO: 65 and SEQ ID NO: 66 specifically hybridize with the gene SEQ ID NO: 22.

Moreover, the above composition may comprise Taqman probes.

The skilled person can easily determine the sequence of said Taqman probes.

The above nucleotides are disclosed in the following table:

| | | | PCR |
|---|---|---|---|
| | | | Product |
| GENE | oligonucleeotide | SEQUENCE | Size (bp) |
| CHI3L1 | Forward primer | GACCACAGGCCATCACAGTCC (SEQ ID NO: 23) | 89 |
| | Reverse primer | TGTACCCCACAGCATAGTCAGTGTT (SEQ ID NO: 24) | |
| IGFBP2 | Forward primer | GGCCCTCTGGAGCACCTCTACT (SEQ ID NO: 25) | 92 |
| | Reverse primer | CCGTTCAGAGACATCTTGCACTGT (SEQ ID NO: 26) | |
| POSTN | Forward primer | GTCCTAATTCCTGATTCTGCCAAA (SEQ ID NO: 27) | 79 |
| | Reverse primer | GGGCCACAAGATCCGTGAA (SEQ ID NO: 28) | |
| HSPG2 | Forward primer | GCCTGGATCTGAACGAGGAACTCTA (SEQ ID NO: 29) | 103 |
| | Reverse primer | AGCTCCCGGACACAGCCTATGA (SEQ ID NO: 30) | |
| BMP2 | Forward primer | CGCAGCTTCCACCATGAAGAATC (SEQ ID NO: 31) | 69 |
| | Reverse primer | GAATCTCCGGGTTGTTTTCCCACT (SEQ ID NO: 32) | |
| COL1A1 | Forward primer | CCTCCGGCTCCTGCTCCTCTT (SEQ ID NO: 33) | 227 |
| | Reverse primer | GGCAGTTCTTGGTCTCGTCACA (SEQ ID NO: 34) | |
| NEK2 | Forward primer | CCCTGTATTGAGTGAGCTGAAACTG (SEQ ID NO: 35) | 101 |
| | Reverse primer | GCTCCTGTTCTTTCTGCTCCAAT (SEQ ID NO: 36) | |
| DLG7 | Forward primer | CCAAATGGAGCAGACTAAGATTGAT (SEQ ID NO: 37) | 67 |
| | Reverse primer | TTGTCTTGGACCAGGTCGGAT (SEQ ID NO: 38) | |
| FOXM1 | Forward primer | GGGAGACCTGTGCAGATGGTGA (SEQ ID NO: 39) | 74 |
| | Reverse primer | TCGAAGCCACTGGATGTTGGAT (SEQ ID NO: 40) | |
| BIRC5 | Forward primer | CCCTTTCTCAAGGACCACCGCATC (SEQ ID NO: 41) | 92 |
| | Reverse primer | CCAGCCTCGGCCATCCGCT (SEQ ID NO: 42) | |
| PLK1 | Forward primer | GCAGATCAACTTCTTCCAGGATCA (SEQ ID NO: 43) | 81 |
| | Reverse primer | CGCTTCTCGTCGATGTAGGTCA (SEQ ID NO: 44) | |
| NKX6-1 | Forward primer | GAGAGGGCTCGTTTGGCCTATT (SEQ ID NO: 45) | 68 |
| | Reverse primer | CGGTTCTGGAACCAGACCTTGA (SEQ ID NO: 46) | |
| NRG3 | Forward primer | AGCCATGTCCAGCTGCAAAATTAT (SEQ ID NO: 47) | 87 |
| | Reverse primer | GCCGACAAAACTTGACTCCATCAT (SEQ ID NO: 48) | |
| BUB1B | Forward primer | ACTACAGTCCCAGCACCGACAAT (SEQ ID NO: 49) | 113 |
| | Reverse primer | TGCTTCGTTGTGGTACAGAAGACTC (SEQ ID NO: 50) | |
| VIM | Forward primer | CTCCCTCTGGTTGATACCCACTC (SEQ ID NO: 51) | 87 |
| | Reverse primer | AGAAGTTTCGTTGATAACCTGTCCA (SEQ ID NO: 52) | |
| TNC | Forward primer | GAGGGTGACCACCACACGCTT (SEQ ID NO: 53) | 73 |
| | Reverse primer | CAAGGCAGTGGTGTCTGTGACATC (SEQ ID NO: 54) | |
| DLL3 | Forward primer | CTCTGCTACCACCGGATGCC (SEQ ID NO: 55) | 99 |
| | Reverse primer | TCAAAGGACCTGGGTGTCTCACTA (SEQ ID NO: 56) | |
| JAG1 | Forward primer | GAAAACGTGCCAGTTAGATGCAA (SEQ ID NO: 57) | 82 |
| | Reverse primer | GCTGGCAATGAGATTCTTACAGGA (SEQ ID NO: 58) | |
| KI67 | Forward primer | ATTGAACCTGCGGAAGAGCTGA (SEQ ID NO: 59) | 105 |
| | Reverse primer | GGAGCGCAGGGATATTCCCTTA (SEQ ID NO: 60) | |
| EZH2 | Forward primer | AACTTCGAGCTCCTCTGAAGCAA (SEQ ID NO: 61) | 97 |
| | Reverse primer | AGCACCACTCCACTCCACATTCT (SEQ ID NO: 62) | |
| BUB1 | Forward primer | CCATTTGCCAGCTCAAGCTAGA (SEQ ID NO: 63) | 102 |
| | Reverse primer | CAGGCCATGTTATTTCCTGGATT (SEQ ID NO: 64) | |
| AURKA | Forward primer | GCATTTCAGGACCTGTTAAGGCTA (SEQ ID NO: 65) | 67 |
| | Reverse Primer | TGCTGAGTCACGAGAACACGTTT (SEQ ID NO: 66) | |

### Kits

The invention also provides kits for use in determining a clinical phenotype (such as prognosis) for a patient afflicted by a glioma, the kit comprising at least one probe specific for a gene or gene product as described above. The preferred combinations of genes or gene products are those described in relation to the methods described herein before.

The probe may be selected from the group consisting of a nucleic acid and an antibody. The kit may also further comprise one or more additional components selected from the group consisting of (i) one or more reference probe(s); (ii) one or more detection reagent(s); (iii) one or more agent(s) for immobilising a polypeptide on a solid support; (iv) a solid support material; (v) instructions for use of the kit or a component(s) thereof in a method described herein.

For example the kit may comprise one or more probes immobilised on a solid support, such as a biochip.

For example the kit may comprise one or more primers suitable for qPCR.

In one embodiment the invention relates to a kit comprising:
oligonucleotides allowing the measure of the expression of the genes of a set comprising at least 3 genes belonging to a group of 22 genes, said 22 genes comprising or being constituted by the respective nucleic acid sequences SEQ ID NO: 1 to 22,
wherein said at least 3 genes comprise or are constituted by the respective nucleic acid sequences SEQ ID NO: 1 to 3, and
a support comprising data regarding the expression value of said at least 3 genes belonging to a group of 22 genes obtained from control patients.

As explained below, “support” in this context may be, for example, computer-readable media, or other data capturing or presenting means.

The invention also relates to a kit comprising:
a composition as defined above, and
a support comprising data regarding the expression value of said at least 3 genes belonging to a group of 22 genes obtained from control patients.

The kit according to the invention is such that it comprises, at least,
oligonucleotides allowing the measure of the expression level of the genes SEQ ID NO: 1, SEQ ID NO: 2 and SEQ ID NO:3, . . . up to SEQ ID NO: 22, and
information regarding the control, or reference, patients that are required to carry out the method according to the invention, said information being on an appropriate support.

Therefore, a minimal format of the kit according to the invention may in one embodiment be:
a pair of oligonucleotides allowing the measure of the expression level of the gene SEQ ID NO: 1, in particular the oligonucleotides SEQ ID NO: 23 and 24,
a pair of oligonucleotides allowing the measure of the expression level of the gene SEQ ID NO: 2, in particular the oligonucleotides SEQ ID NO: 25 and 26,
a pair of oligonucleotides allowing the measure of the expression level of the gene SEQ ID NO: 3, in particular the oligonucleotides SEQ ID NO: 27 and 28, and
a support containing information regarding Qci, Ji, V₁i, V₂i, T1 and T2 values as defined above.

A most advantageous kit according to the invention comprises:
a pair of oligonucleotides allowing the measure of the expression level of the gene SEQ ID NO: 1, in particular the oligonucleotides SEQ ID NO: 23 and 24,
a pair of oligonucleotides allowing the measure of the expression level of the gene SEQ ID NO: 2, in particular the oligonucleotides SEQ ID NO: 25 and 26,
a pair of oligonucleotides allowing the measure of the expression level of the gene SEQ ID NO: 3, in particular the oligonucleotides SEQ ID NO: 27 and 28,
a pair of oligonucleotides allowing the measure of the expression level of the gene SEQ ID NO: 4, in particular the oligonucleotides SEQ ID NO: 29 and 30,
a pair of oligonucleotides allowing the measure of the expression level of the gene SEQ ID NO: 5, in particular the oligonucleotides SEQ ID NO: 31 and 32,
a pair of oligonucleotides allowing the measure of the expression level of the gene SEQ ID NO: 6, in particular the oligonucleotides SEQ ID NO: 33 and 34,
a pair of oligonucleotides allowing the measure of the expression level of the gene SEQ ID NO: 7, in particular the oligonucleotides SEQ ID NO: 35 and 36,
a pair of oligonucleotides allowing the measure of the expression level of the gene SEQ ID NO: 8, in particular the oligonucleotides SEQ ID NO: 37 and 38,
a pair of oligonucleotides allowing the measure of the expression level of the gene SEQ ID NO: 9, in particular the oligonucleotides SEQ ID NO: 39 and 40,
a pair of oligonucleotides allowing the measure of the expression level of the gene SEQ ID NO: 10, in particular the oligonucleotides SEQ ID NO: 41 and 42,
a pair of oligonucleotides allowing the measure of the expression level of the gene SEQ ID NO: 11, in particular the oligonucleotides SEQ ID NO: 43 and 44,
a pair of oligonucleotides allowing the measure of the expression level of the gene SEQ ID NO: 12, in particular the oligonucleotides SEQ ID NO: 45 and 46,
a pair of oligonucleotides allowing the measure of the expression level of the gene SEQ ID NO: 13, in particular the oligonucleotides SEQ ID NO: 47 and 48,
a pair of oligonucleotides allowing the measure of the expression level of the gene SEQ ID NO: 14, in particular the oligonucleotides SEQ ID NO: 49 and 50,
a pair of oligonucleotides allowing the measure of the expression level of the gene SEQ ID NO: 15, in particular the oligonucleotides SEQ ID NO: 51 and 52,
a pair of oligonucleotides allowing the measure of the expression level of the gene SEQ ID NO: 16, in particular the oligonucleotides SEQ ID NO: 53 and 54,
a pair of oligonucleotides allowing the measure of the expression level of the gene SEQ ID NO: 17, in particular the oligonucleotides SEQ ID NO: 55 and 56,
a pair of oligonucleotides allowing the measure of the expression level of the gene SEQ ID NO: 18, in particular the oligonucleotides SEQ ID NO: 57 and 58,
a pair of oligonucleotides allowing the measure of the expression level of the gene SEQ ID NO: 19, in particular the oligonucleotides SEQ ID NO: 59 and 60,
a pair of oligonucleotides allowing the measure of the expression level of the gene SEQ ID NO: 20, in particular the oligonucleotides SEQ ID NO: 61 and 62,
a pair of oligonucleotides allowing the measure of the expression level of the gene SEQ ID NO: 21, in particular the oligonucleotides SEQ ID NO: 63 and 64,
a pair of oligonucleotides allowing the measure of the expression level of the gene SEQ ID NO: 22, in particular the oligonucleotides SEQ ID NO: 65 and 66, and
a support containing information regarding Qci, Ji, V₁i, V₂i, T1 and T2 values as defined above.

Appropriate support comprised in the kit according to the invention can be:
a diskette, a CD-rom, an USB device, or any other device liable to contain pro-gram for computer that have to be implemented in the memory of a computer, containing information regarding Qci, Ji, V₁i, V₂i, T1 and T2 values,
a sheet (paper, carton . . . ) reproducing the information regarding Qci, Ji, V₁i, V₂i, T1 and T2 values, or referring, for instance, to an online software or website, said software or website containing, or compiling, information regarding Qci, Ji, V₁i, V₂i, T1 and T2 values.

The above examples of support are not limitative.

In one advantageous embodiment, the invention relates to the kit as defined above, wherein said support comprises the following data, for measurement with the PCR technique:
when the expression level of the genes SEQ ID NO: 1-3 is measured

| 3 genes | Qci | Ji | V1i | V2i | T1 | T2 |
|---|---|---|---|---|---|---|
| SEQ ID NO: 1 | 9.8895 | 3.5040 | −0.26557206 | 0.5975371 | 0.421766 | 1.4522384 |
| SEQ ID NO: 2 | 10.7617 | 2.8662 | −0.18905578 | 0.4253755 | | |
| SEQ ID NO: 3 | 4.8934 | 4.6331 | −0.04256449 | 0.0957701 | | |

when the expression level of the genes SEQ ID NO: 1-7 is measured

| 7 genes | Qci | Ji | V1i | V2i | T1 | T2 |
|---|---|---|---|---|---|---|
| SEQ ID NO: 1 | 9.8895 | 3.5040 | −0.309811118 | 0.697075015 | 0.4468138 | 1.5790433 |
| SEQ ID NO: 2 | 10.7617 | 2.8662 | −0.233294833 | 0.524913374 | | |
| SEQ ID NO: 3 | 4.8934 | 4.6331 | −0.086803548 | 0.195307982 | | |
| SEQ ID NO: 4 | 8.6122 | 2.5811 | −0.011870396 | 0.026708392 | | |
| SEQ ID NO: 5 | 10.0616 | 2.5943 | 0.008475628 | −0.019070162 | | |
| SEQ ID NO: 6 | 9.1961 | 3.4356 | −0.003268925 | 0.007355082 | | |
| SEQ ID NO: 7 | 7.0401 | 2.5542 | −0.003223563 | 0.007253016 | | |

when the expression level of the genes SEQ ID NO: 1-9 is measured

| 9 genes | Qci | Ji | V1i | V2i | T1 | T2 |
|---|---|---|---|---|---|---|
| SEQ ID NO: 1 | 9.8895 | 3.5040 | −0.331889301 | 0.746750927 | 0.4631175 | 1.6615805 |
| SEQ ID NO: 2 | 10.7617 | 2.8662 | −0.255373016 | 0.574589285 | | |
| SEQ ID NO: 3 | 4.8934 | 4.6331 | −0.10888173 | 0.244983893 | | |
| SEQ ID NO: 4 | 8.6122 | 2.5811 | −0.033948579 | 0.076384303 | | |
| SEQ ID NO: 5 | 10.0616 | 2.5943 | 0.03055381 | −0.068746073 | | |
| SEQ ID NO: 6 | 9.1961 | 3.4356 | −0.025347108 | 0.057030993 | | |
| SEQ ID NO: 7 | 7.0401 | 2.5542 | −0.025301745 | 0.056928927 | | |
| SEQ ID NO: 8 | 6.7866 | 3.1202 | −0.013802309 | 0.031055196 | | |
| SEQ ID NO: 9 | 7.4768 | 2.7594 | −0.002251371 | 0.005065584 | | |

when the expression level of the genes SEQ ID NO: 1-10 is measured

| 10 genes | Qci | Ji | V1i | V2i | T1 | T2 |
|---|---|---|---|---|---|---|
| SEQ ID NO: 1 | 9.8895 | 3.5040 | −0.37621105 | 0.84647485 | 0.509496 | 1.896372 |
| SEQ ID NO: 2 | 10.7617 | 2.8662 | −0.29969476 | 0.67431321 | | |
| SEQ ID NO: 3 | 4.8934 | 4.6331 | −0.15320348 | 0.34470782 | | |
| SEQ ID NO: 4 | 8.6122 | 2.5811 | −0.07827032 | 0.17610823 | | |
| SEQ ID NO: 5 | 10.0616 | 2.5943 | 0.07487556 | −0.16847 | | |
| SEQ ID NO: 6 | 9.1961 | 3.4356 | −0.06966885 | 0.15675492 | | |
| SEQ ID NO: 7 | 7.0401 | 2.5542 | −0.06962349 | 0.15665285 | | |
| SEQ ID NO: 8 | 6.7866 | 3.1202 | −0.05812405 | 0.13077912 | | |
| SEQ ID NO: 9 | 7.4768 | 2.7594 | −0.04657312 | 0.10478951 | | |
| SEQ ID NO: 10 | 8.4759 | 2.9469 | −0.04169181 | 0.09380658 | | |

when the expression level of the genes SEQ ID NO: 1-16 is measured

| 16 genes | Qci | Ji | V1i | V2i | T1 | T2 |
|---|---|---|---|---|---|---|
| SEQ ID NO: 1 | 9.8895 | 3.5040 | −0.398289229 | 0.896150764 | 0.540277 | 2.052201 |
| SEQ ID NO: 2 | 10.7617 | 2.8662 | −0.321772944 | 0.723989123 | | |
| SEQ ID NO: 3 | 4.8934 | 4.6331 | −0.175281658 | 0.394383731 | | |
| SEQ ID NO: 4 | 8.6122 | 2.5811 | −0.100348507 | 0.225784141 | | |
| SEQ ID NO: 5 | 10.0616 | 2.5943 | 0.096953738 | −0.218145911 | | |
| SEQ ID NO: 6 | 9.1961 | 3.4356 | −0.091747036 | 0.206430831 | | |
| SEQ ID NO: 7 | 7.0401 | 2.5542 | −0.091701673 | 0.206328765 | | |
| SEQ ID NO: 8 | 6.7866 | 3.1202 | −0.080202237 | 0.180455034 | | |
| SEQ ID NO: 9 | 7.4768 | 2.7594 | −0.068651299 | 0.154465422 | | |
| SEQ ID NO: 10 | 8.4759 | 2.9469 | −0.063769996 | 0.143482491 | | |
| SEQ ID NO: 11 | 8.4640 | 2.1597 | −0.020277623 | 0.045624651 | | |
| SEQ ID NO: 12 | 5.5556 | 2.3964 | −0.01079938 | 0.024298604 | | |
| SEQ ID NO: 13 | 9.2268 | 3.1865 | 0.008786792 | −0.019770281 | | |
| SEQ ID NO: 14 | 7.4760 | 2.6144 | −0.006607988 | 0.014867974 | | |
| SEQ ID NO: 15 | 16.4164 | 2.8714 | −0.006204653 | 0.013960469 | | |
| SEQ ID NO: 16 | 7.4201 | 3.3385 | −0.003597575 | 0.008094544 | | |

when the expression level of the genes SEQ ID NO: 1-22 is measured

| 22 genes | Qci | Ji | V1i | V2i | T1 | T2 |
|---|---|---|---|---|---|---|
| SEQ ID NO: 1 | 9.8895 | 3.5040 | −0.442610974 | 0.995874691 | 0.6255484 | 2.4838871 |
| SEQ ID NO: 2 | 10.7617 | 2.8662 | −0.366094689 | 0.82371305 | | |
| SEQ ID NO: 3 | 4.8934 | 4.6331 | −0.219603403 | 0.494107658 | | |
| SEQ ID NO: 4 | 8.6122 | 2.5811 | −0.144670252 | 0.325508068 | | |
| SEQ ID NO: 5 | 10.0616 | 2.5943 | 0.141275483 | −0.317869838 | | |
| SEQ ID NO: 6 | 9.1961 | 3.4356 | −0.136068781 | 0.306154758 | | |
| SEQ ID NO: 7 | 7.0401 | 2.5542 | −0.136023419 | 0.306052692 | | |
| SEQ ID NO: 8 | 6.7866 | 3.1202 | −0.124523982 | 0.28017896 | | |
| SEQ ID NO: 9 | 7.4768 | 2.7594 | −0.112973044 | 0.254189348 | | |
| SEQ ID NO: 10 | 8.4759 | 2.9469 | −0.108091741 | 0.243206417 | | |
| SEQ ID NO: 11 | 8.4640 | 2.1597 | −0.064599368 | 0.145348578 | | |
| SEQ ID NO: 12 | 5.5556 | 2.3964 | −0.055121125 | 0.124022531 | | |
| SEQ ID NO: 13 | 9.2268 | 3.1865 | 0.053108537 | −0.119494208 | | |
| SEQ ID NO: 14 | 7.4760 | 2.6144 | −0.050929734 | 0.114591901 | | |
| SEQ ID NO: 15 | 16.4164 | 2.8714 | −0.050526398 | 0.113684396 | | |
| SEQ ID NO: 16 | 7.4201 | 3.3385 | −0.04791932 | 0.107818471 | | |
| SEQ ID NO: 17 | 11.9663 | 3.4954 | 0.030451917 | −0.068516814 | | |
| SEQ ID NO: 18 | 11.3260 | 2.2250 | −0.029802867 | 0.067056452 | | |
| SEQ ID NO: 19 | 9.2557 | 3.1583 | −0.014836187 | 0.033381421 | | |
| SEQ ID NO: 20 | 8.4543 | 2.5087 | −0.010433641 | 0.023475692 | | |
| SEQ ID NO: 21 | 6.9780 | 4.4847 | −0.002903001 | 0.006531752 | | |
| SEQ ID NO: 22 | 7.2556 | 2.6921 | −0.002374696 | 0.005343066 | | |

In one advantageous embodiment, the invention relates to the kit as defined above, wherein said support comprises the following data, for measurement with the DNA CHIP technique:
when the expression level of the genes SEQ ID NO: 1-3 is measured

| 3 genes | Qci | Ji | V1i | V2i | T1 | T2 |
|---|---|---|---|---|---|---|
| SEQ ID NO: 1 | 8.1111 | 3.5040 | −0.26557206 | 0.5975371 | 0.421766 | 1.4522384 |
| SEQ ID NO: 2 | 8.6287 | 2.8662 | −0.18905578 | 0.4253755 | | |
| SEQ ID NO: 3 | 6.0748 | 4.6331 | −0.04256449 | 0.0957701 | | |

when the expression level of the genes SEQ ID NO: 1-7 is measured

| 7 genes | Qci | Ji | V1i | V2i | T1 | T2 |
|---|---|---|---|---|---|---|
| SEQ ID NO: 1 | 8.1111 | 3.5040 | −0.309811118 | 0.697075015 | 0.4468138 | 1.5790433 |
| SEQ ID NO: 2 | 8.6287 | 2.8662 | −0.233294833 | 0.524913374 | | |
| SEQ ID NO: 3 | 6.0748 | 4.6331 | −0.086803548 | 0.195307982 | | |
| SEQ ID NO: 4 | 7.2020 | 2.5811 | −0.011870396 | 0.026708392 | | |
| SEQ ID NO: 5 | 9.2810 | 2.5943 | 0.008475628 | −0.019070162 | | |
| SEQ ID NO: 6 | 9.1734 | 3.4356 | −0.003268925 | 0.007355082 | | |
| SEQ ID NO: 7 | 5.0310 | 2.5542 | −0.003223563 | 0.007253016 | | |

| 9 genes | Qci | Ji | V1i | V2i | T1 | T2 |
|---|---|---|---|---|---|---|
| SEQ ID NO: 1 | 8.1111 | 3.5040 | −0.331889301 | 0.746750927 | 0.4631175 | 1.6615805 |
| SEQ ID NO: 2 | 8.6287 | 2.8662 | −0.255373016 | 0.574589285 | | |
| SEQ ID NO: 3 | 6.0748 | 4.6331 | −0.10888173 | 0.244983893 | | |
| SEQ ID NO: 4 | 7.2020 | 2.5811 | −0.033948579 | 0.076384303 | | |
| SEQ ID NO: 5 | 9.2810 | 2.5943 | 0.03055381 | −0.068746073 | | |
| SEQ ID NO: 6 | 9.1734 | 3.4356 | −0.025347108 | 0.057030993 | | |
| SEQ ID NO: 7 | 5.0310 | 2.5542 | −0.025301745 | 0.056928927 | | |
| SEQ ID NO: 8 | 5.1660 | 3.1202 | −0.013802309 | 0.031055196 | | |
| SEQ ID NO: 9 | 5.1174 | 2.7594 | −0.002251371 | 0.005065584 | | |

when the expression level of the genes SEQ ID NO: 1-9 is measured

| 10 genes | Qci | Ji | V1i | V2i | T1 | T2 |
|---|---|---|---|---|---|---|
| SEQ ID NO: 1 | 8.1111 | 3.5040 | −0.37621105 | 0.84647485 | 0.509496 | 1.896372 |
| SEQ ID NO: 2 | 8.6287 | 2.8662 | −0.29969476 | 0.67431321 | | |
| SEQ ID NO: 3 | 6.0748 | 4.6331 | −0.15320348 | 0.34470782 | | |
| SEQ ID NO: 4 | 7.2020 | 2.5811 | −0.07827032 | 0.17610823 | | |
| SEQ ID NO: 5 | 9.2810 | 2.5943 | 0.07487556 | −0.16847 | | |
| SEQ ID NO: 6 | 9.1734 | 3.4356 | −0.06966885 | 0.15675492 | | |
| SEQ ID NO: 7 | 5.0310 | 2.5542 | −0.06962349 | 0.15665285 | | |
| SEQ ID NO: 8 | 5.1660 | 3.1202 | −0.05812405 | 0.13077912 | | |
| SEQ ID NO: 9 | 5.1174 | 2.7594 | −0.04657312 | 0.10478951 | | |
| SEQ ID NO: 10 | 6.3898 | 2.9469 | −0.04169181 | 0.09380658 | | |

when the expression level of the genes SEQ ID NO: 1-10 is measured

| 10 genes | Qci | Ji | V1i | V2i | T1 | T2 |
|---|---|---|---|---|---|---|
| SEQ ID NO: 1 | 8.1111 | 3.5040 | −0.37621105 | 0.84647485 | 0.509496 | 1.896372 |
| SEQ ID NO: 2 | 8.6287 | 2.8662 | −0.29969476 | 0.67431321 | | |
| SEQ ID NO: 3 | 6.0748 | 4.6331 | −0.15320348 | 0.34470782 | | |
| SEQ ID NO: 4 | 7.2020 | 2.5811 | −0.07827032 | 0.17610823 | | |
| SEQ ID NO: 5 | 9.2810 | 2.5943 | 0.07487556 | −0.16847 | | |
| SEQ ID NO: 6 | 9.1734 | 3.4356 | −0.06966885 | 0.15675492 | | |
| SEQ ID NO: 7 | 5.0310 | 2.5542 | −0.06962349 | 0.15665285 | | |
| SEQ ID NO: 8 | 5.1660 | 3.1202 | −0.05812405 | 0.13077912 | | |
| SEQ ID NO: 9 | 5.1174 | 2.7594 | −0.04657312 | 0.10478951 | | |
| SEQ ID NO: 10 | 6.3898 | 2.9469 | −0.04169181 | 0.09380658 | | |

when the expression level of the genes SEQ ID NO: 1-16 is measured

| 16 genes | Qci | Ji | V1i | V2i | T1 | T2 |
|---|---|---|---|---|---|---|
| SEQ ID NO: 1 | 8.1111 | 3.5040 | −0.398289229 | 0.896150764 | 0.540277 | 2.052201 |
| SEQ ID NO: 2 | 8.6287 | 2.8662 | −0.321772944 | 0.723989123 | | |
| SEQ ID NO: 3 | 6.0748 | 4.6331 | −0.175281658 | 0.394383731 | | |
| SEQ ID NO: 4 | 7.2020 | 2.5811 | −0.100348507 | 0.225784141 | | |
| SEQ ID NO: 5 | 9.2810 | 2.5943 | 0.096953738 | −0.218145911 | | |
| SEQ ID NO: 6 | 9.1734 | 3.4356 | −0.091747036 | 0.206430831 | | |
| SEQ ID NO: 7 | 5.0310 | 2.5542 | −0.091701673 | 0.206328765 | | |
| SEQ ID NO: 8 | 5.1660 | 3.1202 | −0.080202237 | 0.180455034 | | |
| SEQ ID NO: 9 | 5.1174 | 2.7594 | −0.068651299 | 0.154465422 | | |
| SEQ ID NO: 10 | 6.3898 | 2.9469 | −0.063769996 | 0.143482491 | | |
| SEQ ID NO: 11 | 8.8992 | 2.1597 | −0.020277623 | 0.045624651 | | |
| SEQ ID NO: 12 | 2.2380 | 2.3964 | −0.01079938 | 0.024298604 | | |
| SEQ ID NO: 13 | 6.9486 | 3.1865 | 0.008786792 | −0.019770281 | | |
| SEQ ID NO: 14 | 6.6286 | 2.6144 | −0.006607988 | 0.014867974 | | |
| SEQ ID NO: 15 | 13.6886 | 2.8714 | −0.006204653 | 0.013960469 | | |
| SEQ ID NO: 16 | 9.2036 | 3.3385 | −0.003597575 | 0.008094544 | | |

when the expression level of the genes SEQ ID NO: 1-22 is measured

| 22 genes | Qci | Ji | V1i | V2i | T1 | T2 |
|---|---|---|---|---|---|---|
| SEQ ID NO: 1 | 8.1111 | 3.5040 | −0.442610974 | 0.995874691 | 0.6255484 | 2.4838871 |
| SEQ ID NO: 2 | 8.6287 | 2.8662 | −0.366094689 | 0.82371305 | | |
| SEQ ID NO: 3 | 6.0748 | 4.6331 | −0.219603403 | 0.494107658 | | |
| SEQ ID NO: 4 | 7.2020 | 2.5811 | −0.144670252 | 0.325508068 | | |
| SEQ ID NO: 5 | 9.2810 | 2.5943 | 0.141275483 | −0.317869838 | | |
| SEQ ID NO: 6 | 9.1734 | 3.4356 | −0.136068781 | 0.306154758 | | |
| SEQ ID NO: 7 | 5.0310 | 2.5542 | −0.136023419 | 0.306052692 | | |
| SEQ ID NO: 8 | 5.1660 | 3.1202 | −0.124523982 | 0.28017896 | | |
| SEQ ID NO: 9 | 5.1174 | 2.7594 | −0.112973044 | 0.254189348 | | |
| SEQ ID NO: 10 | 6.3898 | 2.9469 | −0.108091741 | 0.243206417 | | |
| SEQ ID NO: 11 | 8.8992 | 2.1597 | −0.064599368 | 0.145348578 | | |
| SEQ ID NO: 12 | 2.2380 | 2.3964 | −0.055121125 | 0.124022531 | | |
| SEQ ID NO: 13 | 6.9486 | 3.1865 | 0.053108537 | −0.119494208 | | |
| SEQ ID NO: 14 | 6.6286 | 2.6144 | −0.050929734 | 0.114591901 | | |
| SEQ ID NO: 15 | 13.6886 | 2.8714 | −0.050526398 | 0.113684396 | | |
| SEQ ID NO: 16 | 9.2036 | 3.3385 | −0.04791932 | 0.107818471 | | |
| SEQ ID NO: 17 | 8.5740 | 3.4954 | 0.030451917 | −0.068516814 | | |
| SEQ ID NO: 18 | 10.7286 | 2.2250 | −0.029802867 | 0.067056452 | | |
| SEQ ID NO: 19 | 4.8529 | 3.1583 | −0.014836187 | 0.033381421 | | |
| SEQ ID NO: 20 | 8.0629 | 2.5087 | −0.010433641 | 0.023475692 | | |
| SEQ ID NO: 21 | 4.8347 | 4.4847 | −0.002903001 | 0.006531752 | | |
| SEQ ID NO: 22 | 6.3091 | 2.6921 | −0.002374696 | 0.005343066 | | |

### Treatment Methods

In one aspect the invention provides a method of treating glioma, which method comprises:

(i) determining a clinical phenotype (such as prognosis) for a patient afflicted by a glioma as described above,

(ii) formulating a therapeutic regime suitable for the treatment of the patient based on the determination at (i); and

(iii) administering said therapeutic regime to said patient.

The terms “treatment” or “therapy” where used herein refer to any administration of a therapeutic (which may or may not be specific for a protein encoded by a gene of the invention described herein) to alleviate the severity of the glioma in the patient, and includes treatment intended to cure the disease, provide relief from the symptoms of the disease and to prevent or arrest the development of the disease in an individual at risk from developing the disease or an individual having symptoms indicating the development of the disease in that individual.

Any sub-titles herein are included for convenience only, and are not to be construed as limiting the disclosure in any way.

The invention will now be further described with reference to the following non-limiting Figures and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

The invention is illustrated by the following example and the following FIGS. 1-5.

### LEGEND TO THE FIGURES

FIG. 1 represents the hierarchical clustering of the training cohort. The initial survival-relevant list of 27 genes was used. Each end line represents a patient. Two branches are separating most of the deceased patients (branch labeled “high risk”, squares) from the mainly alive, low risk patients.

Y-axis represents the dendrogram height; ▪ represents dead patient; ▴ represents alive patient.

FIG. 2 represents the comparison of the overall survival groups generated by hierarchical clustering (black lines; p<2.8e-10) and the OMS classification (grey lines; P<0.018) in the training cohort. Kaplan-Meier curves are plotted for each classification groups and the significance of survival differences is calculated using a log-rank test. Y-axis represents the cumulative survival; X-axis represents the time expressed in months

FIG. 3: Dissimilarities between molecular groups of the training cohort. Assessed by the distance matrix between samples of the training cohort using the expression of the initial 27 genes list. Two regions (similar when darker) clearly group the “Low risk” (LR-1. in the figure) survivors and the “High risk” (HR-2. in the figure), mostly deceased patients.

FIG. 4: Optimization of the predictor length and misclassification errors. The length and the number of errors were plotted as a function of the threshold of the training phase of the PAM algorithm. A number of 22 genes corresponds to the lowest number (0 here) of errors (left-most rectangle ▪) and down to 3 genes keeps the misclassification error under 5% (small rectangle at right ). ∘ represents training error.

X-axis represents threshold.

FIGS. 5A-F represent the comparison of the overall survival groups generated by prediction and the OMS classification in the validation cohort. Kaplan-Meier curves are plotted for each classification groups and the significance of survival differences is calculated using a log-rank test. X-axis represent time in months; Y-axis represent cumulated survival

FIG. 5A represents the Kaplan-Meier curves of the 22 genes of the predictor (black lines; p<2e-14) compared to the WHO prediction (grey lines).

FIG. 5B represents the Kaplan-Meier curves of the 16 genes of the predictor (black lines; p<5.9e-13) compared to the WHO prediction (grey lines).

FIG. 5C represents the Kaplan-Meier curves of the 10 genes of the predictor (black lines; p<2.3e-12) compared to the WHO prediction (grey lines).

FIG. 5D represents the Kaplan-Meier curves of the 9 genes of the predictor (black lines; p<1.4e-8) compared to the WHO prediction (grey lines).

FIG. 5E represents the Kaplan-Meier curves of the 7 genes of the predictor (black lines; p<5.4e-6) compared to the WHO prediction (grey lines).

FIG. 5F represents the Kaplan-Meier curves of the 3 genes of the predictor (black lines; p<1.6e-5) compared to the WHO prediction (grey lines).

### EXAMPLES

All the mathematical and statistical analysis have been realised with the free softwares R version 2.11.1 (http://www.R-project.org) and Bioconductor, version 2.2 [Gentleman R C, et al. Genome Biol. 2004; 5(10):R80].

### Building the Classification on the Training Cohort

### 1/ Gene Choice

A preliminary study made with a limited number of patients has allows the Inventors to identify 38 genes among 380 significantly involved during the low grade glioma progression.

The expression of these genes has been quantified by PCR with oligonucleotides with a control (reference) first cohort of 65 patients well documented (global survival, WHO classification, anatomopathologic information . . . ). This cohort represents the training cohort.

For all the genes, the expression signals obtained by PCR were normalized with the signal of expression of the TBP protein, according to the following formula:

$$Qri = {log}_{2} ( \frac{Si}{Sc} \times 1000 ) ,$$

wherein Si represents the signal obtained for a gene i, and Sc represent the signal obtained for TBP.

For each of the genes, the application of the Cox proportional hazards model (Cox regression) has allowed the Inventors to obtain a gene list ordered by decreasing significant probability.

Applying to that list a Benjamini and Hochberg [Benjamini et al. Journal of the Royal Statistical Society Series B. 1995; 57(1):289-300] multiple testing correction at 5% eliminate 11 genes among the 38 genes used initially. The remaining 27 genes are represented in the following table 4:

| | | | |
|---|---|---|---|
| | | Chromosome | |
| Gene | Probe set | banding | Description^{$} |

Poor prognosis

| | | | |
|---|---|---|---|
| AURKA | 208079_s_at | 20q13.2-q13.3 | serine/threonine kinase 6 |
| BIRC5 | 202095_s_at | 17q25 | baculoviral IAP repeat-containing 5 |
| | | | (survivin) |
| BUB1 | 209642_at | 2q14 | BUB1 budding uninhibited by |
| | | | benzimidazoles 1 homolog (yeast) |
| BUB1B | 203755_at | 15q15 | BUB1 budding uninhibited by |
| | | | benzimidazoles 1 homolog beta (yeast) |
| CHI3L1 | 209396_s_at | 1q32.1 | chitinase 3-like 1 (cartilage glycoprotein-39) |
| COL1A1 | 1556499_s_at | 17q21.3-q22.1 | collagen; type I; alpha 1 |
| DLG7 | 203764_at | 14q22.3 | discs; large homolog 7 (Drosophila) |
| EZH2 | 203358_s_at | 7q35-q36 | enhancer of zeste homolog 2 (Drosophila) |
| FOXM1 | 214148_at | 12p13 | Forkhead box M1 |
| HSPG2 | 201655_s_at | 1p36.1-p34 | heparan sulfate proteoglycan 2 (perlecan) |
| IGFBP2 | 202718_at | 2q33-q34 | insulin-like growth factor binding protein 2; |
| | | | 36 kDa |
| JAG1 | 209099_x_at | 20p12.1-p11.23 | jagged 1 (Alagille syndrome) |
| KI67 | 212020_s_at | 10q25-qter | antigen identified by monoclonal antibody |
| | | | Ki-67 |
| NEK2 | 204641_at | 1q32.2-q41 | NIMA (never in mitosis gene a)-related |
| | | | kinase 2 |
| NKX6-1 | 221366_at | 4q21.2-q22 | NK6 transcription factor related; locus 1 |
| | | | (Drosophila) |
| PLK1 | 1555900_at | 16p12.1 | Polo-like kinase 1 (Drosophila) |
| POSTN | 210809_s_at | 13q13.3 | periostin; osteoblast specific factor |
| PROM1 | 204304_s_at | 4p15.32 | prominin 1 |
| SMO | 218629_at | 7q32.3 | smoothened homolog (Drosophila) |
| TIMELESS | 203046_s_at | 12q12-q13 | timeless homolog (Drosophila) |
| TNC | 201645_at | 9q33 | tenascin C (hexabrachion) |
| VIM | 201426_s_at | 10p13 | vimentin |

Good prognosis

| | | | |
|---|---|---|---|
| APOD | 201525_at | 3q26.2-qter | apolipoprotein D |
| BMP2 | 205289_at | 20p12 | bone morphogenetic protein 2 |
| DLL3 | 219537_x_at | 19q13 | delta-like 3 (Drosophila) |
| NRG3 | 229233 at | 10q22-q23 | neuregulin 3 |
| TACSTD1 | 201839_s_at | 2p21 | tumor-associated calcium signal transducer |
| | | | 1 |

^{$}Affymetrix annotations

Table 4 represents the twenty-seven genes and corresponding probe sets significant in univariate Cox model of overall survival in training cohort with multiple testing corrections.

In general terms, and as described herein, overexpression of APOD, BMP2, DLL3, NRG3 and TACSTD1 may be associated with good prognosis, while overexpression of the remaining genes in Table 1 may be associated with poor prognosis.

### 2/ Training Classes Selection

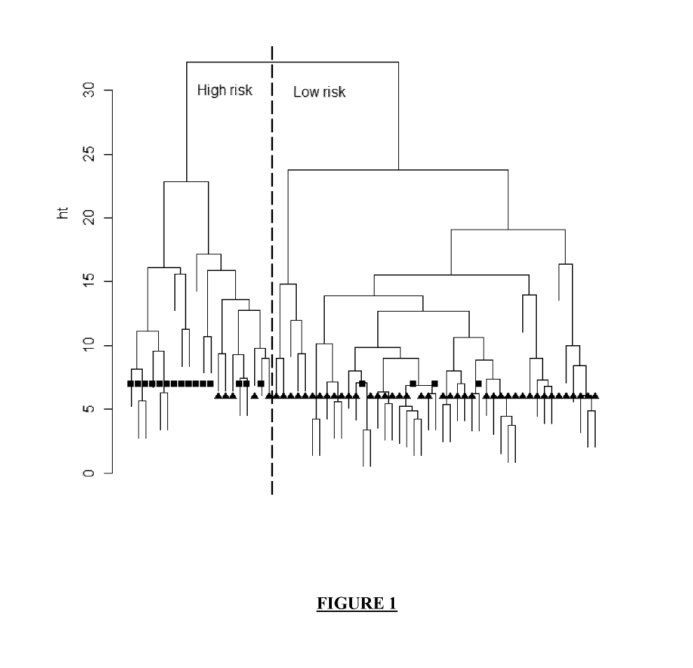

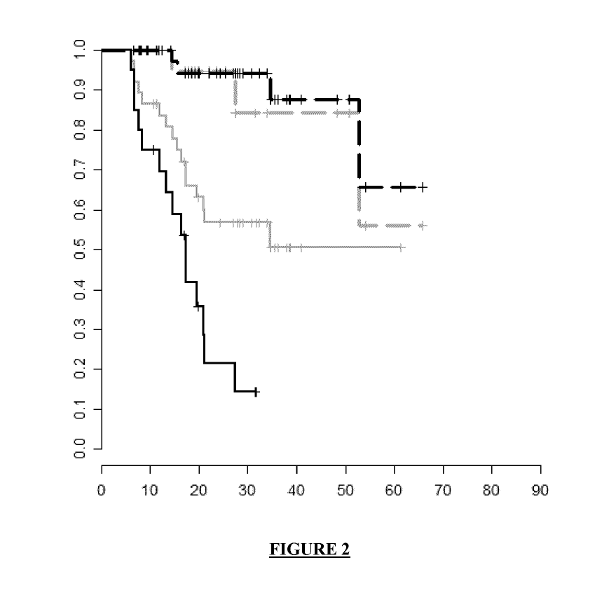

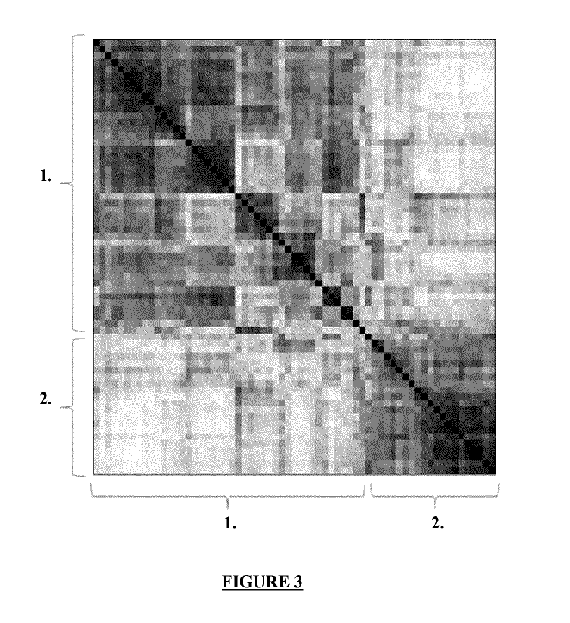

An unsupervised hierarchical clustering (HC) was performed on the PCR expression signal of the 27 OS-relevant genes after normalization on the mean value of each gene over the cohort. Normalization values are recorded for further use with any new patient in the same PCR conditions. As shown on FIG. 1, samples split into two main clusters of 20 and 45 patients. Survival analysis between those groups revealed that 75% (15/20) of patients are deceased in the “High-risk” group compared to only less than 9% (4/45) in the “Low-risk” group. The duration of survival in the latter group is much longer as demonstrated by the Kaplan-Meier curves comparing training classes (black, FIG. 2). The survival curves (grey) for the grade II and III WHO classification in the same cohort were superimposed on the same figure. Strikingly different log-rank tests between classifications are reported in the upper part of Table 5. Dissimilarities between groups are assessed by the distance matrix using the R-package “HOPACH” [van der Loan M and Pollard K. Journal of Statistical Planning and Inference. 2003; 117:275-303]. FIG. 3 again depicts two groups (similarities in blue) clearly separating the “Low risk” (LR)/survivors from the “High risk” (HR)/deceased patients.

Table 5 represents the differential survival analysis of intermediate grade glioma on training and validation cohorts

| | Prognosis | Patient | Event | % | % | Log-rank | % Survival | Median |
|---|---|---|---|---|---|---|---|---|
| Cohort | group | number | number | patient | event | P-value* | at 24 mo | survival (mo) |
| Training | OMS grade 2 | 28 | 3 | 43 | 11 | 0.018 | 95 | NR^{$} |
| | OMS grade 3 | 37 | 16 | 57 | 43 | | 57 | NR |
| | HC^{†} class LR | 45 | 4 | 69 | 9 | 2.8E−10 | 94 | NR |
| | HC class HR | 20 | 15 | 31 | 75 | | 21 | 17.3 |
| Validation | OMS grade 2 | 24 | 16 | 23 | 67 | NS^{#} | 65 | 45.2 |
| | OMS grade 3 | 80 | 72 | 77 | 90 | (0.48) | 60 | 37.9 |
| | PAM^{‡} class LR | 69 | 55 | 66 | 80 | 2.0E−14 | 82 | 72.5 |
| | PAM class HR | 35 | 33 | 34 | 94 | | 18 | 13.2 |

*On one degree of freedom
^{$}Not reached
^{†}Hierarchical Clustering Low (LR) or High (HR) Risk
^{#}Not significant at a 5% risk
^{‡}Prediction Analysis for Microarray Low (LR) or High (HR) Risk

### Building the Classifier on the Training Cohort

### 1/ Predictor Training

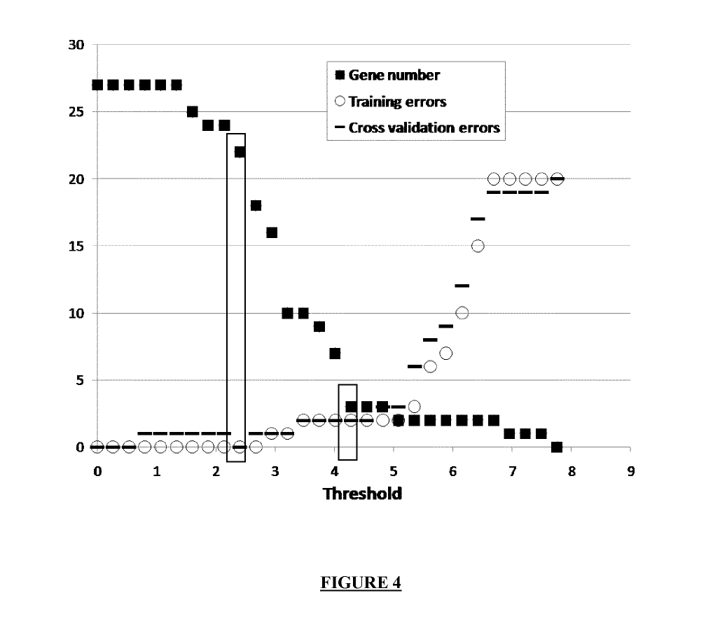

The “pamr” R-package (PAM, prediction analysis for microarray) [Tibshirani R, et al. Proceedings of the National Academy of Sciences of the United States of America. 2002; 99(10):6567-6572] was applied to normalized expression values of the 27 genes between the two prognosis groups selected above in the training cohort. This prediction method is based on “shrunken centroids”, with the “threshold optimization” option (adapted shrinkage thresholds). A 10-times cross validation allows selecting a threshold with a minimal misclassification error rate in training confusion matrices. FIG. 4 displays the number of genes and the respective error rates as a function of the selected threshold. Here, the minimal error rate occurs with a minimal number of 22 out of the initial 27 used for training. The gene list sorted by decreasing scores is depicted in Table 6.

Table 6 represents the twenty-two genes predicting for risk classification in a prediction analysis for microarrays on the training cohort clusters (sorted by score)

Class score

| | Class LR | Class HR |
|---|---|---|
| Gene | Low risk | High risk |
| CHI3L1 | −0.4426 | 0.9959 |
| IGFBP2 | −0.3661 | 0.8237 |
| POSTN | −0.2196 | 0.4941 |
| HSPG2 | −0.1447 | 0.3255 |
| BMP2 | 0.1413 | −0.3179 |
| COL1A1 | −0.1361 | 0.3062 |
| NEK2 | −0.136 | 0.3061 |
| DLG7 | −0.1245 | 0.2802 |
| FOXM1 | −0.113 | 0.2542 |
| BIRC5 | −0.1081 | 0.2432 |
| PLK1 | −0.0646 | 0.1453 |
| NKX6-1 | −0.0551 | 0.124 |
| NRG3 | 0.0531 | −0.1195 |
| BUB1B | −0.0509 | 0.1146 |
| VIM | −0.0505 | 0.1137 |
| TNC | −0.0479 | 0.1078 |
| DLL3 | 0.0305 | −0.0685 |
| JAG1 | −0.0298 | 0.0671 |
| KI67 | −0.0148 | 0.0334 |
| EZH2 | −0.0104 | 0.0235 |
| BUB1 | −0.0029 | 0.0065 |
| AURKA | −0.0024 | 0.0053 |

This constitutes the list to use for prediction of clinical classification of any new patient. But this figure also shows that one can use only the first 3 genes with a slight increase of errors for a similar result (crossing of easy/efficient curves). On the contrary, using the two first genes rapidly increases the error rate and should be avoided. Tables 7 depict confusion matrices in both error-stringent and ease-of-use situations.

Tables 7 represent the confusion matrices (training cohort)

Table 7A represents the 22 genes predictor

| | Prediction | Prediction | Class error |
|---|---|---|---|
| | LR class | HR class | rate |
| Training | | | |
| Low risk (LR) class | 45 | 0 | 0 |
| High risk (HR) class | 0 | 20 | 0 |
| Cross | | | |
| validation | | | |
| Low risk (LR) class | 45 | 0 | 0 |
| High risk (HR) class | 0 | 20 | 0 |

Global error rate = 0

Table 7B represents the 3 genes predictor

| | Prediction | Prediction | Class error |
|---|---|---|---|
| | LR class | HR class | rate |
| Training | | | |
| Low risk (LR) class | 44 | 1 | 0.022 |
| High risk (HR) class | 1 | 19 | 0.05 |
| Cross | | | |
| validation | | | |
| Low risk (LR) class | 44 | 1 | 0.022 |
| High risk (HR) class | 1 | 19 | 0.05 |

Global error rate = 0.031

### 2/ Predictor Validation

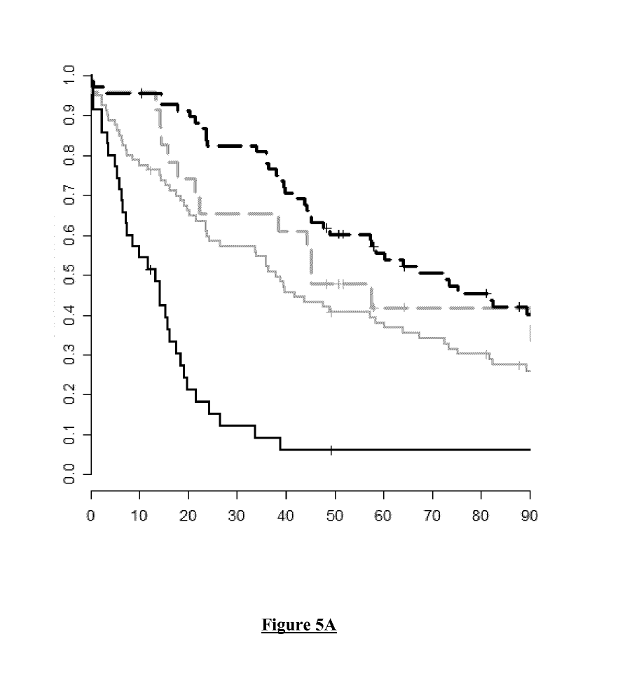

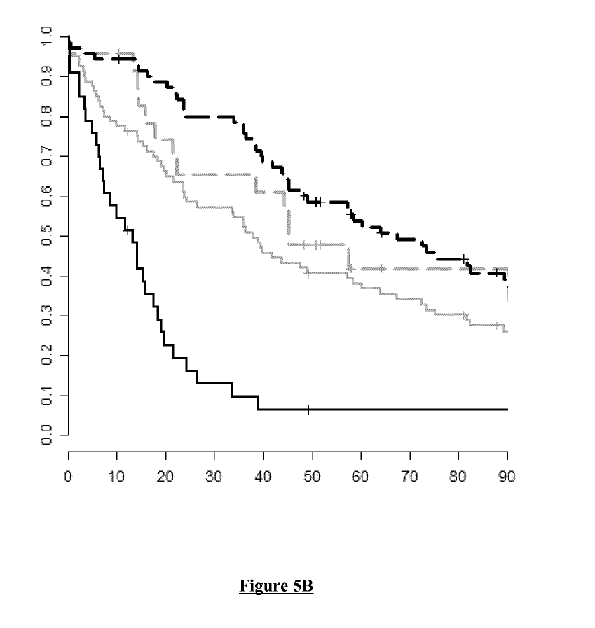

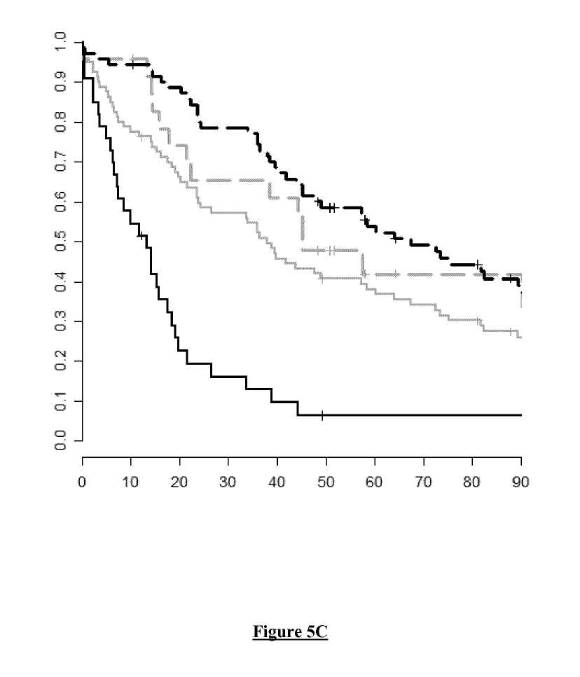

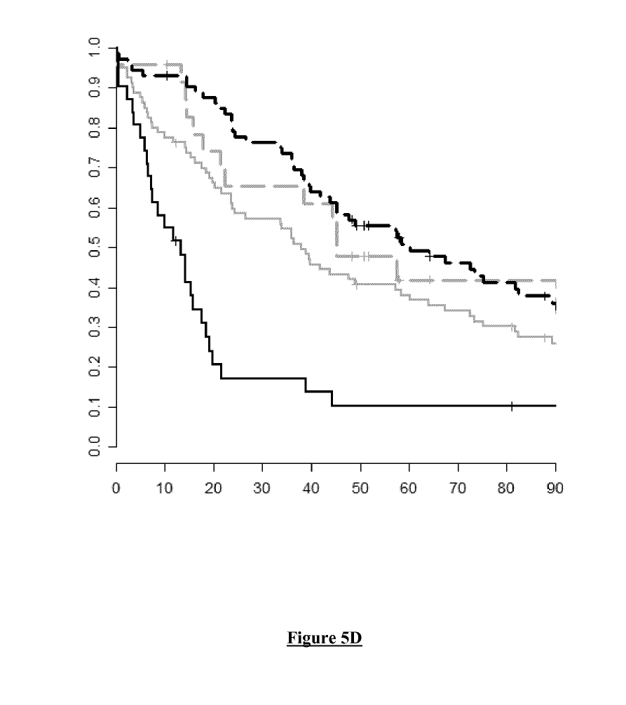

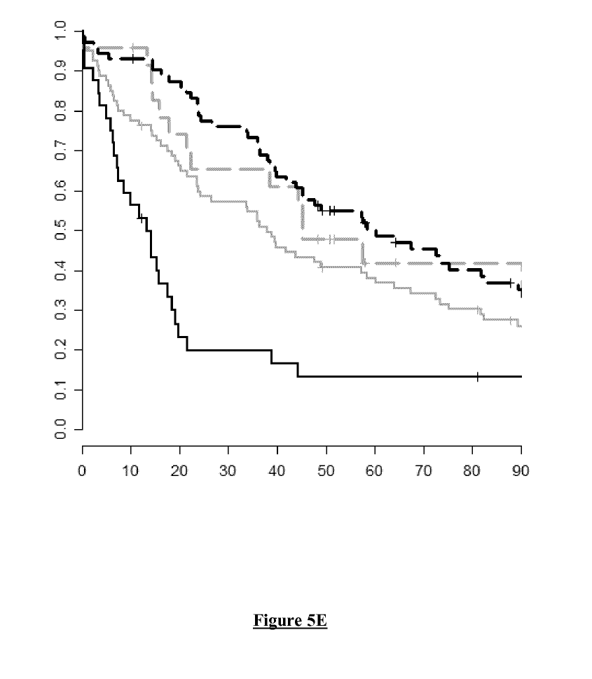

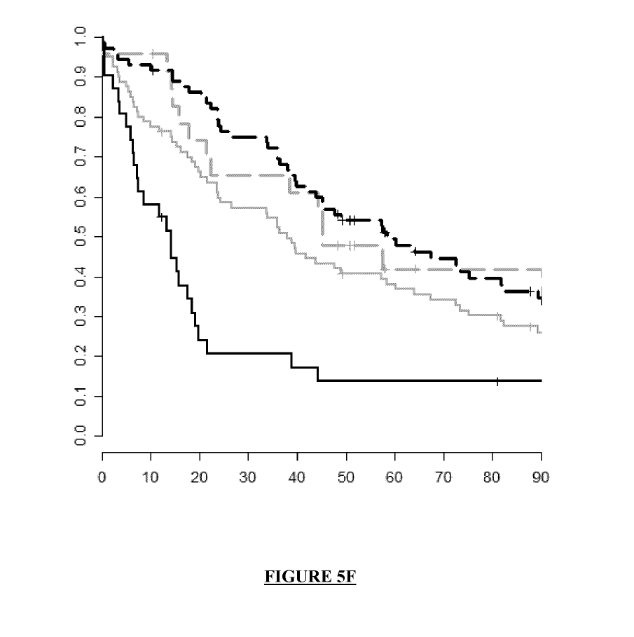

Validation was performed on an independent cohort (Netherlands) of 104 patients with a follow-up of more than 20 years, fully documented for clinical data of overall survival and WHO classification II and Ill grades. For each of these patients, mRNA was purified at diagnosis and hybridized on a Affymetrix U133Plus2.0 chip (^{˜}55,000 pan-genomic probes). Raw files of expression values from chip scans are retrieved along with clinical data (GEO, accession number GSE16011) as published. CEL files are normalized according to the GCRMA [Wu Z, et al. Journal of the American Statistical Association. 2004; 99(8):909-917] method, providing the log₂ of expression value for each probe. We then extracted the 22 probes corresponding to the 22 genes selected during the training phase (listed in Table 4 above). Those values are normalized on the mean value of each probe over the 104 samples. Normalization values are recorded for further use with any new patient in identical conditions, namely same type of chip normalized with the GCRMA parameters from the test cohort using a recent modification (http://code.google.com/p/gep-r/downloads/list) of the incremental preprocessing of the R-package “docval”[Kostka D and Spang R. PloS Comput biol. 2008; 4:e22]. Validation is performed using the “pamr.predict” method of the PAM package PAM, predicting the risk classes Low-LR ou High-HR respectively to differentiate from former WHO “grade II” et “grade III” for the 104 patients of the test cohort. The proportion of high risk patients is 34%, very similar to the one of the training cohort (31%). The strength of the predictor is evaluated by a log-rank test between the two classes survival. Table 5 above (lower part) displays a very significant difference (P≦2×10⁻¹⁴), while WHO classification for this cohort is not even significantly correlated to survival. The Kaplan-Meier curves (FIGS. 5 A-F) illustrate the high-risk classification as a function of the number of predictor genes selected. Finally, the power of the 22 genes predictor compared to conventional WHO classification is illustrated in Table 8, comparing both methods in uni and multivariate Cox analysis.

Furthermore, the dependency of the predictor classification to commonly used grade 2/3 glioma prognostic factors (1p19q loss of heterozygosity, IDH1 gene mutation and EGFR gene amplification) was analyzed using the validation cohort for which these molecular data were available.

As expected the absence of 1p19q codeletion or the amplification of EGFR presented a significant higher risk of poor survival in univariate analysis. However the absence of IDH1 mutation was not associated with a poor outcome in this cohort. In multivariate analysis of each factor and the PAM prediction, only EGFR amplification remained an independent prognostic factor (Table 8). Finally, when testing all prognostic factors together, only PAM classification remained significant.

TABLE 8
Uni- and multivariate Cox model analysis applied to prognosis
groups 30 for overall survival of grade II and III gliomas

| | |
|---|---|
| Training cohort | Validation cohort |

| | | | | |
|---|---|---|---|---|
| Score | HR^{$} | P-value | HR | P-value |

Univariate Cox model

| | | | | |
|---|---|---|---|---|
| WHO | 4.1 | 0.028 | 1.2 | NS^{#} |
| HC/PAM* | 26.2 | 1.7E−05 | 5.8 | 2.2E−12 |
| 1p19q no codeletion | — | — | 1.9 | 0.015 |
| IDH1 no mutation | — | — | 1.1 | NS |
| EGFR amplification | — | — | 4.0 | 3.5E−04 |

Multivariate Cox model

| | | | | |
|---|---|---|---|---|
| HC/PAM | 23.3 | 4.5E−05 | 6.0 | 4.7E−12 |
| WHO | 2.3 | NS | 0.8 | NS |
| PAM | — | — | 9.7 | 5.5E−09 |
| 1p19q no codeletion | — | — | 1.4 | NS |
| PAM | — | — | 6.1 | 1.9E−09 |
| IDH1 no mutation | — | — | 0.7 | NS |
| PAM | — | — | 4.7 | 2.4E−06 |
| EGFR amplification | — | — | 2.7 | 0.015 |
| PAM | — | — | 12.1 | 1.2E−05 |
| WHO | — | — | 0.8 | NS |
| Ip19q no codeletion | — | — | 1.6 | NS |
| IDH1 no mutation | — | — | 1.0 | NS |
| EGFR amplification | — | — | 1.2 | NS |

*HC: training; PAM: predicted validation
^{$}Hazard ratio
^{#}Not significant at a 5% risk

### External Evaluation of a New Patient

Using our method to classify any new patient implies to measure the expression of the 22 genes list by either PCR or microarray technologies, in standardized procedures using the values recorded at the training step to normalize data. Exporting our predictive model should allow an external practitioner to easily calculate the survival risk and therefore the new classification from expression data. For this, successive steps, as illustrated in Table 9, are the following:
1 Centering data on the recorded mean corresponding to the measurement method (PCR, GCRMA/docval normalized microarray)
2 Scaling in reducing to standard deviation of centroids
3 Product of the centered-reduced expression value of each gene by its distance to the class centroid
4 Summing those products
5 Subtracting training baseline to get each class score
6 determine the class with the highest score.
Steps 1 and 2 are data adjustment, steps 3 and 4 can be reduced to the following equation (the gene name represents the adjusted expression level):
Low-risk class score=(BMP2×1.141275)+(NRG3×0.053109)+ . . .
High-risk class score=(BMP2×−0.317870)+(NRG3×−0.119494)+ . . .
After subtraction of the class baseline, those scores are compared to assess the right class to the highest one.
All the preceding operations (from PCR or microarray incremental normalization to classification decision are automated through uploading the expression files to a diagnosis and prognosis website already created for other pathologies (PrognoWeb, https://gliserv.montp.inserm.fr).

Table 9 represents the parameters and risk calculation method to externalize a 22 genes prediction for intermediate grade gliomas

| Provided parameters | | Name | Value | Genes | BMP2 | DLL3 | . . . | NKX6-1 | JAG1 |
|---|---|---|---|---|---|---|---|---|---|
| Centering | Mean 65 samples PCR | A | | | 10.061631 | 11.966334 | . . . | 5.555587 | 11.325967 |
| | Mean 104 samples | B | | | 9.281011 | 8.573953 | . . . | 2.237999 | 10.728599 |
| Scaling | Standard deviation | C | | | 2.594295 | 3.495403 | . . . | 2.396387 | 2.225014 |
| Shrunken | centroids_1 | D | | | 0.141275 | 0.030452 | . . . | — | −0.029803 |
| centroids | centroids_2 | E | | | −0.317870 | −0.068517 | . . . | 0.124023 | 0.067056 |
| Baseline | base_score_1 | F | 0.625548 | | | | | | |
| | base_score_2 | G | 2.483887 | | | | | | |

| New patient (e.g. G533) | | Name | Value | Calculatio | BMP2 | DLL3 | . . . | NKX6-1 | JAG1 |
|---|---|---|---|---|---|---|---|---|---|
| Sample | w expression | H | Input from PCR/Array | | [symbol] | [symbol] | . . . | [symbol] | [symbol] |
| | centered expression | J | | H-A or H-B | 3.400425 | 0.049893 | . . . | — | −1.071109 |
| | scaled centered | K | | J/C | 1.310732 | 0.014274 | . . . | — | −0.481394 |
| | gene_score_1 | L | | K*D | 0.185174 | 0.000435 | . . . | 0.001247 | 0.014347 |
| | gene_score_2 | M | | K*E | −0.416642 | −0.000978 | . . . | — | −0.032281 |
| | sum_score_1 | N | 2.412382 | sum(L) | | | | | |
| | sum_score_2 | P | — | sum(M) | | | | | |
| | class_score_1 | Q | 1.786834 | N-F | | | | | |
| | class_score_2 | R | — | M-G | | | | | |
| | Risk class | Low = 1 | 1 | 1 if Q > R | | | | | |
| | | High = 2 | | 2 if Q ≦ R | | | | | |

Bold: Given parameters
Italic: Input from new sample test
Normal: Calculated or deduced

1-15. (canceled)

16. Method for determining, in vitro or ex vivo, from a biological sample of a subject afflicted by a WHO grade 2 or grade 3 glioma, the survival prognosis of said patient,
said method comprising:
determining the quantitative expression value Qi for each gene of a set comprising at least 3 genes belonging to a group of 22 genes, said 22 genes comprising or being constituted by the respective nucleic acid sequences SEQ ID NO: 1 to 22, wherein said at least 3 genes comprise or are constituted by the respective nucleic acid sequences SEQ ID NO: 1 to 3,
establishing
a first product P₁i for each of said at least 3 genes, between the respective Qi values obtained above for each said at least 3 genes and a first value V₁i, and
a second product P₂i for each of said at least 3 genes, between the respective Qi values obtained above for each said at least 3 genes and a second value V₂i,
wherein
said first value Vii corresponds to the shrunken centroïd value for a gene i obtained from reference patients having a WHO grade 2 or grade 3 glioma, said reference patients having a median survival higher than 4 years, and
said second value V2i corresponds to the shrunken centroïd value for a gene i obtained from reference patients having a WHO grade 2 or grade 3 glioma, said reference patients having a median survival lower than 4 years,
said patients having a WHO grade 2 or grade 3 glioma with a median survival lower or higher than 4 years belonging to a reference cohort of patients afflicted by either a WHO grade 2 or a WHO grade 3 glioma,
determining the survival rate of said patient as follows:
if the sum of the P₁i products of each of said at least 3 genes is higher than the sum of the P₂i products of each of said at least 3 genes, then said subject has a median survival higher than 4 years, and
if the sum of the P₁i products of each of said at least 3 genes is lower than or equal to the sum of the P₂i products of each of said at least 3 genes, then said subject has a median survival lower than 4 years.

17. Method according to claim 16, wherein said set comprise at least 7 genes belonging to said group of 22 genes, said at least 7 genes comprising or being constituted by the respective nucleic acid sequences SEQ ID NO: 1 to 7.

18. Method according to claim 16, wherein said set comprise at least 9 genes belonging to said group of 22 genes, said at least said at least 9 genes comprising or being constituted by the respective nucleic acid sequences SEQ ID NO: 1 to 9.

19. Method according to claim 16, wherein said set consists of all the genes of said group of 22 genes.

20. Method according to claim 16, wherein
if N1>N2, then said patient has a median survival higher than 4 years, preferably from 4 to 10 years, more preferably from 5 to 8 years, in particular about 6 years, and
if N1≦N2, then said patient has a median survival lower than 4 years, preferably from 0.5 to 3.5 years, more preferably from 0.5 to 2 years, in particular about 1 year, wherein
$$N 1 = {\sum }_{i = 1}^{n} ( {P}_{1} i ) - {T}_{1} = ( {\sum }_{i = 1}^{n} ( ( \frac{Qri - Qci}{Ji} ) \times {V}_{1} i ) ) - {T}_{1} ,$$
n varying from 3 to 22, and
$$N 2 = {\sum }_{i = 1}^{n} ( {P}_{2} i ) - {T}_{2} = ( {\sum }_{i = 1}^{n} ( ( \frac{Qri - Qci}{Ji} ) \times {V}_{2} i ) ) - {T}_{2} ,$$
n varying from 3 to 22,
wherein
Qri represents the quantitative raw expression value measured for a gene i in the biological sample of said subject, and
Qci represents the mean of the quantitative expression values obtained for said gene i from each patient of said control cohort of patient afflicted by a WHO grade 2 or grade 3 glioma,
Ji represents the standard deviation of the centroid values obtained for said gene i from each patient of said control cohort of patient afflicted by a WHO grade 2 or grade 3 glioma,
V₁i corresponds to the shrunken centroïd value for said gene i obtained from control patients having a WHO grade 2 or grade 3 glioma with a median survival higher than 4 years,
V₂i corresponds to the shrunken centroïd value for said gene i obtained from control patients having a WHO grade 2 or grade 3 glioma with a median survival lower than 4 years,
T1 corresponds to the training baseline value for control patients having a WHO grade 2 or grade 3 glioma with a median survival higher than 4 years, and
T2 corresponds to the training baseline value for control having a WHO grade 2 or grade 3 glioma with a median survival lower than 4 years.

21. Method according to claim 16, wherein the quantitative expression value Qi for a gene i is measured by quantitative techniques chosen among qRT-PCR and DNA Chip.

22. Method according to claim 20, relates to the method as defined above, wherein, when the quantitative technique is DNA CHIP, Qci values for a gene i are as follows:

| Genes | Qci |
|---|---|
| SEQ ID NO: 1 | 8.1111 |
| SEQ ID NO: 2 | 8.6287 |
| SEQ ID NO: 3 | 6.0748 |
| SEQ ID NO: 4 | 7.2020 |
| SEQ ID NO: 5 | 9.2810 |
| SEQ ID NO: 6 | 9.1734 |
| SEQ ID NO: 7 | 5.0310 |
| SEQ ID NO: 8 | 5.1660 |
| SEQ ID NO: 9 | 5.1174 |
| SEQ ID NO: 10 | 6.3898 |
| SEQ ID NO: 11 | 8.8992 |
| SEQ ID NO: 12 | 2.2380 |
| SEQ ID NO: 13 | 6.9486 |
| SEQ ID NO: 14 | 6.6286 |
| SEQ ID NO: 15 | 13.6886 |
| SEQ ID NO: 16 | 9.2036 |
| SEQ ID NO: 17 | 8.5740 |
| SEQ ID NO: 18 | 10.7286 |
| SEQ ID NO: 19 | 4.8529 |
| SEQ ID NO: 20 | 8.0629 |
| SEQ ID NO: 21 | 4.8347 |
| SEQ ID NO: 22 | 6.3091 |

23. Method according to claim 20, wherein, when the quantitative technique is qRT-PCR, Qci values for a gene i are as follows:

| Genes | Qci |
|---|---|
| SEQ ID NO: 1 | 9.8895 |
| SEQ ID NO: 2 | 10.7617 |
| SEQ ID NO: 3 | 4.8934 |
| SEQ ID NO: 4 | 8.6122 |
| SEQ ID NO: 5 | 10.0616 |
| SEQ ID NO: 6 | 9.1961 |
| SEQ ID NO: 7 | 7.0401 |
| SEQ ID NO: 8 | 6.7866 |
| SEQ ID NO: 9 | 7.4768 |
| SEQ ID NO: 10 | 8.4759 |
| SEQ ID NO: 11 | 8.4640 |
| SEQ ID NO: 12 | 5.5556 |
| SEQ ID NO: 13 | 9.2268 |
| SEQ ID NO: 14 | 7.4760 |
| SEQ ID NO: 15 | 16.4164 |
| SEQ ID NO: 16 | 7.4201 |
| SEQ ID NO: 17 | 11.9663 |
| SEQ ID NO: 18 | 11.3260 |
| SEQ ID NO: 19 | 9.2557 |
| SEQ ID NO: 20 | 8.4543 |
| SEQ ID NO: 21 | 6.9780 |
| SEQ ID NO: 22 | 7.2556 |

24. Composition comprising oligonucleotides allowing the quantitative measure of the expression level of the genes of a set comprising at least 3 genes belonging to a group of 22 genes, said 22 genes comprising or being constituted by the respective nucleic acid sequences SEQ ID NO: 1 to 22,
wherein said at least 3 genes comprise or are constituted by the respective nucleic acid sequences SEQ ID NO: 1 to 3.

25. Composition according to claim 24, wherein said set comprise at least 7 genes belonging to said group of genes, said at least 7 genes comprising or being constituted by the respective nucleic acid sequences SEQ ID NO: 1 to 7.

26. Composition according to claim 24, wherein said set comprise at least 9 genes belonging to a said group of 22 genes, said at least 9 genes comprising or being constituted by the respective nucleic acid sequences SEQ ID NO: 1 to 9.

27. Composition according to claim 24, wherein said set consists of all the genes of said group of 22 genes.

28. Composition according to claim 24, wherein said composition comprise at least a pair of oligonucleotides allowing the measure of the expression of the genes of said set of genes belonging to said group of 22 genes.

29. Composition according to claim 28, wherein said composition comprises at least the oligonucleotides SEQ ID NO: 23-28, or at least the oligonucleotides SEQ ID NO: 23-40, or at least the oligonucleotides SEQ ID NO: 23-42, or at least the oligonucleotides SEQ ID NO: 23-54, chosen among the group consisting of the oligonucleotides SEQ ID NO: 23-66, or said composition comprising the oligonucleotides SEQ ID NO: 23-66.

30. Kit comprising:
oligonucleotides allowing the measure of the expression of the genes of a set comprising at least 3 genes belonging to a group of 22 genes, said 22 genes comprising or being constituted by the respective nucleic acid sequences SEQ ID NO: 1 to 22,
wherein said at least 3 genes comprise or are constituted by the respective nucleic acid sequences SEQ ID NO: 1 to 3, and
a support comprising data regarding the expression value of said at least 3 genes belonging to a group of 22 genes obtained from control patients.
